(19) United States
(12) Patent Application Publication
(10) Pub. No.: US 2026/0263647 A1
(43) Pub. Date: Sep. 10, 2026

(54) DEVELOPING AN ANTI-HUMAN CD45 IMAGING PROBE FOR NONINVASIVE DETECTION OF HUMAN IMMUNE CELLS

(71) Applicant: Dana-Farber Cancer Institute, Inc., Boston, MA (US)

(72) Inventors: Mohammad RASHIDIAN, Boston, MA (US); Ali Salehi FARID, Boston, MA (US); Jennifer ROWLEY, Boston, MA (US)

(73) Assignee: Dana-Farber Cancer Institute, Inc., Boston, MA (US)

(21) Appl. No.: 19/165,925

(22) PCT Filed: Mar. 15, 2024
(86) PCT No.: PCT/US2024/020183

§ 371 Date: Sep. 16, 2025

Related U.S. Application Data
(60) Provisional application No. 63/452,818, filed on Mar. 17, 2023.

(51) Int. Cl. A61K 51/10; C07K 16/28

(52) U.S. Cl. CPC A61K 51/1093; A61K 51/1018; C07K 16/289; C07K 2317/526; C07K 2317/569; C07K 2317/64; C07K 2317/92

(57) ABSTRACT

Disclosed are radiolabeled tracers, diagnostic compositions, methods of imaging and diagnosing of inflammation, and methods of treating inflammation.

### CROSS-REFERENCE TO RELATED APPLICATIONS

This application claims the benefit of priority under 35 U.S.C. § 119(e) to U.S. Provisional Application No. 63/452,818, filed Mar. 17, 2023, which is incorporated herein by reference in its entirety.

### GOVERNMENT LICENSE RIGHTS

This invention was made with government support under grant number K22 CA226040 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Mar. 5, 2024, is named 52095_764001WO_ST.xml and is 38 KB bytes in size.

### BACKGROUND OF THE INVENTION

Diseases characterized by inflammation were once thought to only be involved in a select group of disorders, such as autoimmune conditions, but are now known to be responsible for over 50% of all deaths worldwide (Roth et al., The Lancet 392(10159):1736-1788 (2018); Furman et al., Nat. Med. 25(12):1822-1832 (2019)). Inflammatory diseases such as cancer, diabetes mellitus, neurodegenerative conditions, ischemic heart disease and other inflammatory cardiomyopathies, inflammatory bowel diseases (IBDs), pulmonary inflammatory diseases, non-alcoholic fatty liver disease, autoimmune conditions, and graph-versus-host disease are growing in incidence globally. Inflammatory cardiomyopathies, such as cardiac sarcoidosis, cellular cardiac transplant rejection, and myocarditis can lead to heart failure, arrhythmias, and death. IBDs can lead to chronic abdominal pain, diarrhea, and malnutrition. Pulmonary inflammatory diseases can lead to fatigue, Extreme restlessness, slow heart rate and eventually, hypercapnia.

Currently, clinical evaluation of chronic inflammatory processes is conducted on an organ-by-organ basis, typically after a patient presents with a new symptom or abnormal finding on imaging or bloodwork. Subsequent diagnostic workups often rely on invasive methods such as biopsy alongside organ-specific serum readouts which have limited sensitivity and specificity. For example, endomyocardial biopsies are the standard approach for the diagnosis and monitoring of inflammatory cardiomyopathies, and endoscopic biopsies are the standard approach in the diagnosis and monitoring of IBDs. Indeed, the entire gastrointestinal (GI) tract (i.e., digestive tissue), from the mouth to the rectum, can succumb to dysregulated inflammatory conditions. For example, upper GI tract diseases like eosinophilic esophagitis, chronic gastroesophageal reflux or a simmering Helicobacter pylori infection can increase risk of esophageal or gastric cancer if not diagnosed and treated (O'Sullivan et al., Expert Rev. Gastroenterol. Hepatol. 8(7):749-760 (2014)). In the lower GI tract, IBDs such as Crohn's disease and ulcerative colitis (UC) can have significant effects on a patient's life, with risk of malnutrition, bowel injury and perforation, colorectal cancer as well as systemic manifestations like decreased bone density and extra-colonic cancers. Like the aforementioned GI diseases, the diagnosis of IBD relies on endoscopic evaluation and biopsy, which is invasive and can lack specificity (Negreanu et al., Therap. Adv. Gastroenterol. 12:1756284819865153, pp. 1-10 (2019)). Endoscopy also aids in differentiating Crohn's from UC, which is challenging given that as much as 20-30% of Crohn's patients present with lesions possibly unreachable by colonoscope (Molinié et al., Gut 53(6):843-848 (2004)). Even after diagnosis, this patient population is highly monitored, requiring endoscopic reassessment with each major change in treatment regimen (Dmochowska et al., Int. J. Mol. Sci. 19 (9): 2471 pp. 1-13 (2018)).

Early detection of the inflammatory disease GVHD remains a pressing clinical dilemma. GVHD is a common immune complication of hematopoietic stem-cell transplantation (HSCT) and is caused by the recognition and destruction of recipient tissues, spanning skin, liver, bowel, and lungs, by donor T cells (Malard et al., Nat. Rev. Dis. Primers 9(1):27 (2023)). GVHD is a leading cause of morbidity and lethality in these patients (Ferrara et al., Lancet 373(9674):1550-1561 (2009); Khoury et al., Haematologica 102(5):958-966 (2017)). The diagnosis methods of combined symptom assessment and biopsy histologic analyses, not only lack sufficient sensitivity and specificity, but also may subject medically fragile patients to invasive procedures like endoscopy or liver biopsy (Malard et al., Nat. Rev. Dis. Primers 9(1):27 (2023); Ross et al., Am J Gastroenterol 103:982-989 (2008); Aslanian et al., Dig. Dis. Sci. 57(3):720-725 (2012)).

These invasive biopsies have significant complication rates and can lack specificity due to uneven disease distribution when an inflammatory disease presents in some areas and not others. For instance, inflammatory cardiomyopathies may present in some areas of the heart but not others (described as “patchy”) and a false-negative biopsy will result when a biopsy is taken from healthy tissue region and not from a diseased tissue region (i.e., a patch).

Imaging-based diagnoses are better able to detect patchy inflammatory diseases, such as cardiomyopathies, pulmonary inflammatory disease, IBDs, and some cancers. Imaging-based techniques include positron emission tomography (PET), single-photon emission computerized tomography (SPECT), and magnetic resonance imaging (MRI). However, common imaging probes (e.g., ¹⁸F-fluorodeoxyglucose (¹⁸FDG)) are not specific for inflammation, have indiscriminate uptake in some tissues, making it susceptible to false positives and unnecessary downstream workup for patients, and is therefore nonideal for imaging inflammatory diseases. Moreover, these probes suffer from physiologic myocardial uptake due to the fact that the myocardium uses either free fatty acid or glucose as a substrate for metabolic energy and will take up ¹⁸FDG. Therefore, imaging specific tissues, especially cardiac tissue, with ¹⁸FDG requires strictly controlled patient preparation. Varied patterns of physiological myocardial uptake, including regional heterogeneity, may still be seen, making it difficult to discern among physiologic uptake, benign conditions causing update, and actual disease. Collectively, these impediments make the heart and myocardium a blind spot for disease diagnosis (Lobert et al., Clin. Radiol. 68:e59-71 (2013); Fukuchi et al., Br. J. Radiol. 80:3-11 (2007)).

Other PET probes targeting immune cells have largely struggled with the opposite issue, namely that their targets have been too specific to make them useful across in many inflammatory diseases. Such probes are meticulously engineered to target specific metabolic pathways, for example, the small molecule probe ¹⁸F-arabinosyl guanine (¹⁸F-AraG) that targets deoxycytidine kinase and mitochondrial deoxyguanosine kinase (Namavari et al., Mol. Imaging Biol. 13(5):812-818 (2011); Levi et al., Cancer Res. 79(13):3455-3465 (2019)). Probes targeting specific molecules have also been developed, for example, ⁶⁸Ga-pentixafor that targets CXCR4 (Lapa et al., Theranostics 7(1):205-212 (2017), Derlin et al., Eur. J. Nucl. Med. Mol. Imaging 45(11):1934-1944 (2018)). Probes tailored to detect immune cell subtypes have also been developed, e.g., antibody fragment-based probes targeting CD8⁺ cytotoxic T cells, CD11b⁺ myeloid cells, and CD206⁺ mannose receptor-expressing anti-inflammatory macrophages (Kist et al., Nat. Med. 28(12):2601-2610 (2022); Farwell et al., J. Nucl. Med. 63(5):720-726 (2022); Rashidian et al., Proc. Natl. Acad. Sci. U.S.A 116(34):16971-16980 (2019)), (Nigam et al., Mol. Imaging Biol. 22(3):685-694 (2020)) (Gondry et al., J. Nucl. Med. 64(9):1378-1384 (2023)).

Therefore, there is an urgent need for a reliable PET probe capable of detecting inflammatory conditions with the balance of sensitivity and specificity required for effective diagnosis and monitoring. This gap underscores the pressing need for a PET probe designed specifically for inflammation detection, which is essential for early diagnosis, precise monitoring of therapeutic responses, and improving patient outcomes.

### SUMMARY OF THE INVENTION

The radiolabeled tracers, diagnostic compositions, methods of detecting inflammation, methods detecting the presence of CD45⁺ immune cells, kits, and methods of treating an inflammatory disease provided herein are expected to address the above need.

A first aspect of the present disclosure is directed to a radiolabeled tracer, containing a) a single chain variable antibody fragment (scFv) that binds CD45, wherein the scFv contains a variable light (VL) domain and a variable heavy (VH) domain connected by a first linker, b) a constant heavy (CH)3 domain connected to the scFv by a second linker, and c) a radioactive isotope detectable via positron emission tomography (PET) or single-photon emission computerized tomography (SPECT), wherein the radioactive isotope is connected to one or more of the VL, VH, and CH3 domains.

Another aspect of the present disclosure is directed to a diagnostic composition containing an effective amount of the radiolabeled tracer and a pharmaceutically acceptable carrier.

A further aspect of the present disclosure is directed to a method of detecting inflammation in a subject. The method entails intravenously administering to a subject in need thereof the diagnostic composition and producing a first image of a tissue of the subject by PET or SPECT imaging, wherein detecting the radioactive isotope in the tissue is an indication of inflammation in the tissue. The disclosed methods are advantageous in that they not only detect the presence of the inflammation, but its severity as well. In some embodiments, the subject is undergoing therapy for the inflammation and the methods are used to assess or monitor the therapy and determine whether it is effective in ameliorating the inflammation.

A further aspect of the present disclosure is directed to a method of detecting the presence of CD45⁺ cells in a tissue of a subject. The method entails intravenously administering to a subject in need thereof, the diagnostic composition, producing a first image of a tissue of the subject by PET or SPECT imaging, wherein detecting the radioactive isotope is an indication of the presence of CD45⁺ immune cells.

Yet another aspect of the present disclosure is directed to a method of treating an inflammatory disease in a subject. The method entails intravenously administering to a subject in need thereof the diagnostic composition, producing a first image of a tissue of the subject by PET or SPECT imaging, wherein detecting the radioactive isotope in the tissue is an indication of inflammation, and if the radioactive isotope is detected in the tissue, administering an appropriate therapy to the subject.

Yet another aspect of the present disclosure is directed to a kit containing a) the diagnostic composition, and b) printed instructions for use in intravenously administering the composition to a subject having or suspected of having inflammation or an inflammatory disease or condition.

The CD45 radiolabeled tracers described herein allow for the detection of immune cells in any organ or tissue in the body. As demonstrated in the examples provided, CD45⁺ immune cells serve as an indication of inflammation and inflammatory disease in several body tissues/organs including cardiac tissue, digestive tissues, lung tissue, the skin, as well as others, because significant numbers of these cells are not present in healthy, non-inflamed tissue. Healthy heart tissue imaged by PET for immune cells (with a radiolabeled tracer specific for cellular metabolism) merely have background levels of radioactive isotope. Similarly, healthy digestive tissue and lung tissue imaged by PET for immune cells also have background levels of radioactive isotope. Imaging a subject with a radiolabeled tracer that binds CD45 provides a non-invasive imaging technique that has excellent tissue penetration, high target affinity, and rapid clearance from the circulation, enabling a sensitive and specific method for detecting and monitoring inflammation. Detection of a CD45-PET signal higher than the background level in an organ, e.g., lung, liver, kidney, or heart, may be indicative of inflammation in that organ or a focal portion thereof, e.g., parts of lung that show a higher signal than the other parts.

Working examples disclosed herein demonstrate that the radiolabeled tracer is a non-invasive, quantitative whole-body modality approach for the detection of inflammation-causing cells in vivo and may be used as a longitudinal diagnostic to follow disease progression or remission over time and in response to treatment.

### BRIEF DESCRIPTION OF THE DRAWINGS

FIGS. 1A-1E are a series of schematic illustrations, SDS-PAGE gel image, line plot, and dot plots showing the development of a radiolabeled tracer that binds CD45. FIG. 1A is a schematic illustration of an antibody fragment that binds human CD45 (CD45 minibody) from which the radiolabeled tracer was derived. FIG. 1B is a schematic illustration of the antibody fragment that binds CD45 linked to a copper-64 radioactive isotope. FIG. 1C is an image of an SDS-PAGE gel showing the anti-CD45-minibody probe expressed in HEK293 cells. FIG. 1D is a line plot showing staining on human PBMCs, using different concentrations of the probe, showed an EC₅₀ of ~10 nM. FIG. 1E is a series of dot plots showing that the probe stains human PBMCs but did not stain human keratinocytes confirming its specificity toward its target molecule.

FIGS. 2A-2B are a series of PET-CT images showing humanized NOD scid gamma (NSG) mice that were injected with human CD45⁺ PBMCs, human CD45-specific radiolabeled tracer, and subjected to PET-CT imaging. FIG. 2A is a PET-CT image of a control NSG mouse 24 h post-injection with ~30 μCi of radiolabeled tracer. FIG. 2B is a series of PET-CT images of humanized NSG mice 24 h post-injection with ~30 μCi of radiolabeled tracer.

FIGS. 3A-3E are a series of schematic illustrations, photographs, line plots, and bar plots showing the visualization of immune cells in vivo with the ⁸⁹Zr-CD45 nanobody PET probe (CD45-PET probe). FIG. 3A is a schematic illustration of CD45 nanobody (~15 kDa) labeled specifically at its C-terminus with a polyethylene glycol (PEG) moiety (20 kDa) and equipped with a deferoxamine (DFO) chelator for binding with an ⁸⁹Zr PET isotope. FIG. 3B is a line plot showing that the PET probe exhibits high-affinity staining of murine splenocytes. FIG. 3C is an image showing that the CD45-PET probe detects immune cell-rich organs of healthy mice. FIG. 3D is a bar plot showing quantification of SUVₘₑₐₙ of CD45-PET signal in healthy mice. FIG. 3E is a bar plot showing decay-corrected gamma radiation assay of the dissected organs of healthy mice.

FIGS. 4A-4J are a series of schematic illustrations, images, dot plots, and bar plots showing CD45-PET signal directly correlates with both degree of inflammatory infiltrate and clinical severity in an acute respiratory distress syndrome model. FIG. 4A is a schematic illustration showing that mice were induced with ARDS. FIG. 4B is a set of in vivo PET-CT images depicting immune organs and lung inflammation in LPS-treated mice. FIG. 4C is a set of in vivo axial images of the lungs with areas of above-background PET signal marked with arrowheads. FIG. 4D is ex vivo organ PET/CT images of mouse lungs. FIGS. 4E and 4F are a set of is a bar plot showing ex vivo organ PET/CT images of each respective mouse's lungs. FIG. 4G is a set of immunohistochemistry (IHC) images showing representative of high CD45 expression in high-dose LPS- and control-treated mice. FIG. 4H is a set of images demonstrating the expression of murine CD45 in selected areas in the 10X panel of FIG. 4G as shown with the box. FIG. 4I is a bar plot showing percentage of positive pixels of CD45 IHC staining and lung SUVₘₑₐₙ. FIG. 4J is a bar plot showing the percentage of weight loss in all groups and lung SUVₘₑₐₙ.

FIGS. 5A-50 are a series of schematic illustrations, images, dot plots, and bar plots showing CD45-PET detection of inflammation in a murine inflammatory bowel disease (IBD) model and outperforms ¹⁸F-FDG-PET in discerning disease severity. FIG. 5A is a schematic illustration showing acute colitis was induced in mice by exposure to 4% w/v dextran sodium sulfate (DSS) or control in drinking water. FIG. 5B is a bar plot showing quantitative in vivo analysis of large bowel signal. FIG. 5C is a bar plot showing quantitative in vivo analysis of rectum signal. FIG. 5D is a set of CD45-PET/CT images showing depicting signal in the large bowel (wedges), appendix (asterisk) and mesenteric lymph nodes (arrow) in DSS-treated mice. FIG. 5E is a set of representative images showing gross dissected large bowels. FIG. 5F is a set of representative ex vivo PET/CT images showing the large bowel of high-signal DSS-treated and control mice. FIGS. 5G and 5H are a set of 10X and 40X hematoxylin & eosin (H&E) images showing bowel sections from areas denoted with brackets in FIG. 5F. FIG. 5I is a dot plot showing quantitative analysis of ex vivo imaging of samples taken for histology. FIG. 5J is a dot plot showing percentage of weight loss in DSS-treated mice. FIG. 5K is a dot plot showing the disease activity score in DSS-treated mice. FIG. 5L is a set of ¹⁸F-FDG PET/CT and PET images showing illustrating large bowel inflammation (wedges), representative for 7-day DSS-treated mice; the bottom panel portrays an axial view of the large bowel. FIG. 5M is a ¹⁸F-FDG PET/CT and PET images showing illustrating the large bowel inflammation (wedges) in the mouse in FIG. 5L, 24 hours later as detected by the CD45-PET probe. Asterisks show appendix; the bottom panel portrays an axial view of the large bowel. FIG. 5N is a dot plot showing a comparison between ¹⁸F-FDG-PET and weight loss in mice. FIG. 5O is a dot plot showing a comparison between ⁸⁹Zr-CD45 PET and weight loss in mice.

FIGS. 6A-6E are a series of schematic illustrations, images, dot plots, and bar plots showing longitudinal CD45-PET imaging visualizes inflammation regression in a murine IBD model. FIG. 6A is a schematic illustration showing an acute colitis model induced in C57BL/6 mice by exposure to 2% w/v DSS or control in water. FIG. 6B is a set of representative images showing DSS-treated mice 8 days after initiation of 2% DSS exposure. Large bowel inflammation (asterisk) and increased mesenteric lymph node uptake (wedge) are clearly visualized. FIG. 6C is a dot plot showing the percentage of weight loss as compared to large bowel SUV. FIG. 6D is a set of representative images showing the DSS-treated mouse show in FIG. 6B, 8 days after recovery on regular water without dexamethasone. Decreased large bowel inflammation (asterisk) and mesenteric lymph node uptake (wedge) are clearly visualized. FIG. 6E is a dot plot showing the percentage of weight loss as compared to change in large bowel SUV.

FIGS. 7A-7I are a series of schematic illustrations, images, line plots, and bar plots showing human CD45-PET probe detects engrafted human immune cells in humanized mice. FIG. 7A is a schematic illustration showing anti-human CD45 antibody fragment (~80 kDa). FIG. 7B is a line plot showing that the human CD45-PET antibody fragment exhibits high-affinity binding to human peripheral blood mononuclear cells (PBMCs). FIG. 7C is a schematic illustration of the experimental setup of MHC I/II deficient NSG mice humanized by intravenous injection of 10×10⁶ human PBMCs and having underwent CD45-PET/CT imaging on Day 21 post-humanization. FIG. 7D is a set of representative day 21 PET/CT and PET images showing humanized (left-sided panels) and non-humanized control (right-sided panels) mice. FIG. 7E is a bar plot showing quantitative analysis of in vivo spleen uptake of the human CD45-PET probe in humanized mice as compared with non-humanized control mice. FIG. 7F is a set of representative ex vivo images of the mice shown in FIG. 7D showing higher human CD45-PET probe signal in the spleen (asterisk) of the humanized mice compared with the non-humanized control. Muscle tissue (M) used as negative control. FIGS. 7G-7H are a set of bar plots showing quantitative analysis of ex vivo and gamma radiation spleen uptake of the human CD45-PET probe in humanized mice as compared with non-humanized control mice. FIG. 7I is a bar plot showing quantitative analysis of in vivo PET signal in the spleen and major non-immune organ systems.

FIGS. 8A-8I are a series of images, dot plots, and bar plots showing whole body visualization of human immune cell infiltration using the human CD45-PET probe. FIG. 8A is a set of representative PET/CT and PET images showing mouse 2 (left-sided panels) and mouse 4 (right-sided panels) on day 21. Clear signal can be seen in the spleen (asterisk). FIG. 8B is a bar plot showing GVHD symptoms scored for each mouse (M1-M4, F1). FIG. 8C is a set of representative repeat PET/CT and PET MIP images showing at day 112 displaying decreased spleen signal (asterisk) and increased snout (Sn), kidney (Ki) and perineum (Per) signal across both mouse 2 and mouse 4. FIG. 8D is a set of representative ex vivo images showing that the lungs of mouse 4 have higher signal than mouse 2, with the reverse observed for colon. FIG. 8E is a line plot showing that all mice that developed one or more GVHD symptoms had a significant decrease in spleen signal between days 21 and 112 after humanization. FIGS. 8F and 8G are a set of line plots showing the change of in vivo PET signal in the colons and lungs of each mouse (M1-M4, F1) between days 21 and 112. FIGS. 8H and 8I are a set of representative images showing human CD45 IHC staining performed on the proximal colons (20X) and right lungs (10X) of mice after in vivo and ex vivo imaging.

FIGS. 9A-9G are a series of schematic illustrations, images, and bar plots showing the synthesis of ⁸⁹Zr-CD45 nanobody PET probe via sortase-mediated site-specific installment of DFO and optimization of image quality via installation of polyethylene glycol (PEG) molecules. FIG. 9A is a schematic illustration depicting sortase-mediated site-specific labeling of the nanobody. FIG. 9B is an image showing SDS-PAGE analyses confirming the generation of the product. Lanes: #1: protein marker, #2: nanobody, #3: DFO-azide labeled nanobody, #4: DFO-labeled nanobody PEGylated with a 20 kDa PEG moiety. FIG. 9C is a line plot showing flow cytometric assessments of CD45 nanobody stained splenocytes. FIG. 9D is a set of representative images showing that PEGylation increases the signal-to-noise and decreases non-specific kidney uptake of the CD45-PET probe. FIG. 9E is a bar plot showing quantified in vivo PET signal from the PEGylation comparison experiment normalized against muscle. FIG. 9F is a set of representative images showing mice pre-injected with CD45 nanobody (VHH), which shows significant decrease in immune organ signal compared to mice with no pre-injection. FIG. 9G is a bar plot showing in vivo spleen SUVₘₑₐₙ quantification compared to mice given no pre-injection.

FIGS. 10A-10F are a series of images, dot plots, and bar plots showing ex vivo PET/CT, gamma radiation assay, and histological assessment validating that the CD45-PET can detect inflammation in vivo in a murine model of ARDS. FIG. 10A is a bar plot showing quantification of results of gamma radiation assay represented as % injected dose per gram (% ID/g) of lungs of mice 30 hours after intranasal administration of LPS or control. FIGS. 10B and 10C are a set of dot plots showing IHC staining for murine CD45⁺ cells were performed on fixed lung samples of high (n=1), low (n=3), and control mice (n=2). The percentage of positively staining pixels per field of view strongly correlated with ex vivo lung SUVₘₑₐₙ and gamma radiation % ID/g. FIG. 10D is a set of representative images showing H&E staining (10X) of representative high dose LPS lung section shows significant inflammatory infiltration that is predominantly neutrophil driven (inset, 40X) as well as collapse of alveoli in comparison to saline control lung section. FIGS. 10E and 10F are a set of dot plots showing correlation between probe uptake and weight loss (used as a marker of clinical severity) was observed in both ex vivo lung SUVₘₑₐₙ and gamma radiation % ID/g.

FIGS. 11A-11H are a series of dot plots and bar plots showing that the CD45-PET detects inflammation in a murine IBD model and staining correlates with disease severity. FIG. 11A is a line plot showing that DSS-treated mice had significantly increased weight loss compared to controls. FIG. 11B is a line plot showing that DSS colitis disease activity index (DAI) was scored every other day, where weight, stool consistency, and bleeding were evaluated and graded based on severity. FIGS. 11C and 11D are a set of bar plots showing quantitative analysis of ex vivo PET images and gamma radiation assay, confirming that DSS-exposed mice had higher large bowel PET signal and probe uptake compared to controls. FIGS. 11E and 11F are a set of dot plots showing quantitative analysis of ex vivo PET images and gamma radiation assay, confirming that large bowel PET signal and probe uptake significantly correlate with percent weight loss from baseline. FIG. 11G is a dot plot showing in vivo PET signal in the rectum correlated with weight loss. FIG. 11H is a dot plot showing in vivo PET signal in the rectum versus disease activity score.

FIGS. 12A-12F are a series of dot plots and bar plots showing that the CD45-PET and ¹⁸F-FDG probes detect inflammation in the large bowel, but only the CD45-PET probe detects rectum inflammation. FIGS. 12A and 12B are a set of plots showing in vivo quantification of ¹⁸F-FDG-PET and ⁸⁹Zr-CD45-PET images in the large bowel. FIGS. 12C and 12D are a set of bar plots showing in vivo quantification of ¹⁸F-FDG-PET and ⁸⁹Zr-CD45-PET images in the rectum. FIGS. 12E and 12F are a set of dot plots showing in vivo quantification of ¹⁸F-FDG-PET and ⁸⁹Zr-CD45-PET images as compared to weight loss.

FIGS. 13A-13I are a series of dot plots, bar plots, and line plots showing CD45-PET imaging detects bowel inflammation in lower dose 2% DSS regimen of IBD model and captures response to treatment to water recovery with and without dexamethasone. FIGS. 13A-13C are a set of bar plots showing large bowel and rectum in vivo CD45-PET signal. FIGS. 13D-13E are a set of bar plots and dot plots showing CD45-PET imaging 8 days after mice were randomized to water recovery alone (n=6) or water recovery with dexamethasone. FIGS. 13F-13G are a set of bar plots showing percent change in SUVₘₑₐₙ between water+dexamethasone and water-alone groups in the large bowel and rectum. FIG. 13H is a bar plot showing weight change from day 8 to day 16 in water+dexamethasone and water-alone groups. FIG. 13I is a dot plot showing percentage of weight gain in regular water-only treated and regular water with dexamethasone-treated groups.

FIGS. 14A-14I are a series of images, bar plots, and dot plots showing that the human CD45-PET probe has low background signal in major non-immune organs. FIG. 14A is an SDS-PAGE image showing the purity and dimeric structure of the human CD45-targeting antibody fragment (minibody); lanes: #1: protein marker, #2: antibody fragment dimer (minibody), #3: antibody fragment monomer after exposure to heat and DTT. FIG. 14B is a dot plot showing quantitative analysis of in vivo PET signal in humanized versus non-humanized mice. FIGS. 14C-14G are a set of dot plots showing comparison of spleen uptake versus uptake in major organ systems. FIGS. 14H-14I are a set of bar plots showing ex vivo and gamma radiation assay analysis of the subsets of humanized and non-humanized mice.

FIGS. 15A-15K are a series of bar plots and line plots showing longitudinal whole body in vivo PET SUVₘₑₐₙ quantification analysis of humanized mice detecting above-background signal increases in major organs at imaging time points of day 21 and day 112.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated to facilitate the understanding of the present disclosure.

As used in the description and the appended claims, the singular forms “a”, “an”, and “the” mean “one or more” and therefore include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “including,” “containing,” or “characterized by,” is inclusive or open-ended and does not exclude additional, unrecited elements or method steps. By contrast, the transitional phrase “consisting of” excludes any element or method step not specified in the claim (or the specific element or method step with which the phrase “consisting of” is associated). The transitional phrase “consisting essentially of” limits the scope of a claim to the specified elements and method or steps and “unrecited elements and method steps that do not materially affect the basic and novel characteristic(s)” of the claimed disclosure.

### Radiolabeled Tracer

In one aspect, the disclosure provides a radiolabeled tracer containing a) a single chain variable antibody fragment (scFv) that binds CD45, wherein the scFv contains a variable light (VL) domain and a variable heavy (VH) domain connected by a first linker, b) a constant heavy (CH) 3 domain connected to the scFv by a second linker, and c) a radioactive isotope detectable via positron emission tomography (PET) or single-photon emission computerized tomography (SPECT), wherein the radioactive isotope is connected to at least one of the VL, VH, and CH3 domains.

CD45 is a hematopoietic lineage-restricted cell-surface molecule that is expressed on all hematopoietic cells except for erythroid cells, platelets, and plasma cells. Non-immune cells including myocytes do not express CD45.

scFv

The scFv may be derived from an antibody that binds CD45. Derivation of antibody fragments (e.g., scFv) from antibodies is known in the art. The term “derived from” as used herein refers to a protein or nucleic acid that originates from another, parent (e.g., naturally occurring or wild type) protein or nucleic acid, that may be identical to the parent, may be a portion or fragment of the parent, or may differ from the parent in terms of sequence. Variants may include amino acid and nucleotide substitutions, insertions, or deletions, respectively. Thus, for example, an amino acid sequence derived from a parent sequence may be identical for a specific range of amino acids of the parent but does not include amino acids outside that specific region.

The scFv may be derived from an anti-CD45 antibody and include, for example, antibody fragments, and derivatives thereof. Amino acid sequences of representative anti-CD45 antibody variable fragments are set forth in Table 1.

TABLE 1
Amino acid sequences of representative anti-CD45 antibody variable fragments

```
Clone BC8 VL domain (SEQ ID NO: 1)
    1 dialtqspas lavslgqrat iscrasksvs tsgysylhwy qqkpgqppkl liylasnles
  61 gvparfsgsg sgtdftlnih pveeedaaty ycqhsrelpf tfgsgtklei k
Clone BC8 VH domain (SEQ ID NO: 2)
    1 evkllesggg lvqpggslkl scaasgfdfs rywmswvrqa pgkglewige inptsstinf
  61 tpslkdkvfi srdnakntly lqmskvrsed talyycargn yyrygdamdy wgqgtsvtvs
121 sak
Ab 4122 VL domain (SEQ ID NO: 3)
    1 divmtqtpas vsepvggtvt imcqasqsis nwlawyqqkp gqppklliyq asklasgvps
  61 rfkgsgsgte ytltisdlec adaatyycqs yydsgsnvff afgggtkvvv e
Ab 4122 VH domain (SEQ ID NO: 4)
    1 lsleesggdl vkpgasltlt ctasgfsfsa gywicwvrqa pgkglewiac tyagrsgsty
  61 yanwvngrft ipktssttvt lqmtslsgad tasyfcargn agvavgalwg pgtlvtvss
Ab 4129 VL domain (SEQ ID NO: 5)
    1 divmtqtpas veaavggtvt incqasqsis swlswyqqkp goppklliyg asnlasgvps
  61 rfsgsgsgtq fsltisdlec adaatyycqs yydsgssvff nigggtkvvv k
Ab 4129 VH domain (SEQ ID NO: 6)
    1 qsleesggdl vkpgasltlt ctasgfsfsa gywicwvrqa pgkglewiac iyagssgsty
  61 yaswakgrft ipktssttvt lqmtsltgad tatyfcargn agvavgalwg pgtlvtvss
Ab 4131 VL domain (SEQ ID NO: 7)
    1 divmtqtpas vsepvggsvt ikcqasqsfy nllawyqqkp gqppklliyd asdlasgvps
  61 rfkgsgsgtd ftltisdlec adaaayycqs adgssyafgg gtevvvk
Ab 4131 VH domain (SEQ ID NO: 8)
    1 qeqleesggg lvkpegsltl tctasgvsfs ssywiywvrq apgkglewia ciytgssgst
  61 yyaswakgrf tvsetssttv tlqmtsltaa dtatyfcara sawtygmdlw gpgtlvtvss
```

In some embodiments, the scFv contains the VH domain that has the amino acid sequence set forth below (SEQ ID NO: 9):

```
    1 qvqlvesggg lvqpggslkl scaasgfdfs
        rywmswvrqa pgkglewige inptsstinf
  61 tpslkdkvfi srdnakntly lomskvrsed
        talyycargn yyrygdamdy wgqgtsvtvs
121 kis
```

In some embodiments, the scFv contains the VL domain that has the amino acid sequence set forth below (SEQ ID NO: 10):

```
  1 divltqspas lavslggrat iscrasksvs
      tsgysylhwy qqkpgqppkl liylasnles
61 gvparfsgsg sgtdftlnih pveeedaaty
      ycqhsrelpf tfgsgtklei k
```

Additional antibodies and antibody fragments that bind CD45 are known in the art. See, e.g., U.S. Pat. Nos. 10,420,851, 10,624,973, and 10,774,152, and U.S. Patent Application Publications 2018/0237521, 2020/0338219, 2021/0371524, and 2022/0062339.

### CH3 Domain

The CH3 domain of the radiolabeled tracer enables dimerization of two scFv-CH3 domain monomers.

In some embodiments, the CH3 domain may be derived from IgA, IgD, IgG, IgM, or IgE. In some embodiments, the dimerization domain is the IgG1 constant heavy 3 (CH3) domain. The amino acid sequence of a representative IgG1 CH3 domain is set forth below (SEQ ID NO: 11):

```
  1 qprepqvytl ppsrdeltkn qvsltclvkg
      fypsdiavew esngqpenny kttppvldsd
61 gsfflysklt vdksrwqqgn vfscsvmhea
      lhnhytqksl slspgk
```

### Linkers

The radiolabeled tracer further contains linkers disposed between components of the radiolabeled tracer. The first linker is disposed between the VH and the VL of the scFv and the second linker is disposed between the scFv and the CH3 domain. In some embodiments, the second linker attaches the CH3 domain to the VL. In some embodiments, the second linker attaches the CH3 domain to the VH.

The first and the second linkers may provide flexibility in terms of allowing the VH and the VL of the scFv to bind CD45. The first and second linkers contain at least four amino acids, the selection of which is not critical.

The first and the second linker may be the same or different. In some embodiments, the first linker, the second linker, or both the first and the second linker has the amino acid sequence GGGX, GGGGX (SEQ ID NO: 12), or GSSGSX (SEQ ID NO: 13), where X is any nucleotide, typically either cysteine (C) or serine(S), or a repeating sequence thereof. In some embodiments, one or both of the first and second linker are a multiple of 4 amino acids in length (e.g., repeats of GGGX). In some embodiments, one or both of the first and second linker are a multiple of 5 amino acids in length (e.g., repeats of SEQ ID NO: 12). In some embodiments, the first linker, the second linker, or both the first and the second linker has the amino acid sequence GGGGS (SEQ ID NO: 14), GSPRG (SEQ ID NO: 15), GGGGSGGGGS (SEQ ID NO: 16), GGGGSGGGGSGGGGS (SEQ ID NO: 17), GGGGSGGGGSGGGGSGGGGS (SEQ ID NO: 18), GGGGSGGGGSGGGGSGGGGSGGGGS (SEQ ID NO: 19), GGGSEPKSPKSADKTHTAP (SEQ ID NO: 20), GSTSGSGKPGSGEGSTKG (SEQ ID NO: 21), KESGSVSSEQLAQFRSLD (SEQ ID NO: 22), EGKSSGSGSESKST (SEQ ID NO: 23), or GSAGSAAGSGEF (SEQ ID NO: 24).

In some embodiments, the first linker has the amino acid sequence GGGGSGGGGSGGGGSGGGGSGGGGS (SEQ ID NO: 19). In some embodiments, the second linker has the amino acid sequence GGGSEPKSPKSADKTHTAP (SEQ ID NO: 20).

In some embodiments, the linker may be derived from IgA, IgD, IgE, IgG, or IgM. In some embodiments, the linker may be derived from the hinge region of CD3ζ, CD4, CD8α, CD28, IgG1, IgG2, or IgG4. Amino acid sequences of additional representative linkers are listed in Table 2.

TABLE 2
Amino acid sequences of
representative linkers

| Linker | Sequence |
|---|---|
| CD3ζ (SEQ ID | QSFGLLDPK |
| NO: 25) | |
| CD4 (SEQ ID | LSEGDKVKMDSRIQVLSRGVNQT |
| NO: 26) | |
| CD8α (SEQ | KPTTTPAPRPPTPAPTIASQPLSK |
| ID NO: 27) | RPEACRPAAGGAVHTRGLDFACDIY |
| CD28 (SEQ | IEVMYPPPYLDNERSNGTIIHVKG |
| ID NO: 28) | KHLCPSPLFPGPSKP |
| IgG1 (SEQ ID | EPKSCDKTHTCPPCPAPELLGG |
| NO: 29) | |
| IgG2 (SEQ ID | ERKCCVECPPCPAPPAAA |
| NO: 30) | |
| IgG4 (SEQ ID | ESKYGPPCPPCPAPEFLGG |
| NO: 31) | |

Further examples of linkers suitable for use in the present radiolabeled tracers are known in the art. See, Klein et al., Protein Eng Des Sel 27:325-30 (2014), U.S. Pat. Nos. 10,882,909 and 11,180,570, and U.S. Patent Application Publication No. 2020/0291354A1.

In some embodiments, the scFv contains a VH, linker, and VL, the amino acid sequence of which is set forth below (SEQ ID NO: 32):

```
    1 qvqlvesggg lvqpggslkl scaasgfdfs
        rywmswvrqa pgkglewige inptsstinf
  61 tpslkdkvfi srdnakntly lomskvrsed
        talyycargn yyrygdamdy wgqgtsvtvs
121 kisggggsgg ggsggggsgg ggsggggssd
        ivltqspasl avslgqrati scrasksvst
181 sgysylhwyq qkpgqppkll iylasnlesg
        vparfsgsgs gtdftlnihp veeedaatyy
241 cqhsrelpft fgsgtkleik
```

In some embodiments, the radiolabeled tracer monomer contains a VH, linker, VL, linker, and CH3 domain, the amino acid sequence of which is set forth below (SEQ ID NO: 33):

| | |
|---|---|
| 1 | qvqlvesggg lvqpggslkl scaasgfdfs |
| | rywmswvrqa pgkglewige inptsstinf |
| 61 | tpslkdkvfi srdnakntly lqmskvrsed |
| | talyycargn yyrygdamdy wgqgtsvtvs |
| 121 | kisggggsgg ggsggggsgg ggsggggssd |
| | ivltqspasl avslgqrati scrasksvst |
| 181 | sgysylhwyq qkpgqppkll iylasnlesg |
| | vparfsgsgs gtdftlnihp veeedaatyy |
| 241 | cqhsrelpft fgsgtkleik gggsepkspk |
| | sadkthtapq prepqvytlp psrdeltknq |
| 301 | vsltclvkgf ypsdiavewe sngqpennyk |
| | ttppvldsdg sfflyskltv dksrwqqgnv |
| 361 | fscsvmheal hnhytqksls lspgk |

### Radioactive Isotope

The tracer may be radiolabeled with one or more radioactive isotopes. The one or more radioactive isotopes enable detection via positron emission tomography (PET) or single-photon emission computerized tomography (SPECT). In some embodiments, the one or more radioactive isotopes are suitable for detection in PET-computed tomography (PET-CT) or combined PET and magnetic resonance imaging (MRI) (PET-MRI).

Representative examples of radioactive isotopes that may be contained in the radiolabeled tracer include Carbon-11 (¹¹C, ~20.4 m half-life), Nitrogen-13 (¹³N, ~10 m half-life), Oxygen-15 (¹⁵O, ~2 m half-life), Gallium-68 (⁶⁸Ga, ~68m half-life), Fluorine-18 (¹⁸F, ~109.7 m half-life), Gallium-66 (⁶⁶Ga, ~9.5 h half-life), Copper-64 (⁶⁴Cu, ~12.7 h half-life), niobium-90 (⁹⁰Nb, ~14.5 h half-life), bromine-76 (¹⁶Br, ~16.2 h half-life), Yttrium-86 (⁸⁶Y, ~17.4 h half-life), Cobalt-55 (⁵⁵Co, ~17.5 h half-life), Terbium-152 (¹⁵²Tb, ~17.5 h half-life), Arsenic-72 (⁷²As, ~20 h half-life), and Germanium-69 (⁶⁹Ge, ~39 h half-life), indium (¹¹¹In ~2.8 day or 67.2 h half-life), zirconium-89 (⁸⁹Zr, ~3.3 day or 78.4 h half-life), iodine-124 (¹²⁴I, ~4.17 day half-life), and Manganese-52 (⁵²Mn, ~5.59 day half-life).

In some embodiments, the radioactive isotope is zirconium-89 (⁸⁹Zr), fluorine-18 (¹⁸F), iodine-124 (¹²⁴I), copper-64 (⁶⁴Cu), gallium-68 (⁶⁸Ga), yttrium-90 (⁹⁰Y), or indium (¹¹¹In).

One or more of the VL, VH, and CH3 domains may be directly or indirectly radiolabeled. In some embodiments, the radioactive isotope is connected directly to an amino acid residue of at least one of the VL, VH, and CH3 domains. In some embodiments, one of the VL, VH, or CH3 domain is radiolabeled. In some embodiments, two of the VL, VH, and CH3 domains are radiolabeled. In some embodiments, all three of the VL, VH, and CH3 domains are radiolabeled. In some embodiments, a domain of the tracer is radiolabeled with more than one radioactive isotope. Direct radiolabeling may be accomplished, for example, through electrophilic substitution at reactive aromatic amino acids (e.g., tyrosine and histidine residues). Direct radiolabeling methods and techniques are known in the art. See, for example, U.S. Pat. Nos. 4,361,544, 4,427,646, 5,985,240, 8,137,540, and 11,298,433, and U.S. Patent Publication Application 2005/0013778.

In some embodiments, one or more radioactive isotopes are connected indirectly to at one or more of the VL, VH, and CH3 domains. In some embodiments, one or more radioactive isotopes are connected indirectly to at least one of the VL, VH, and CH3 domains by a chelator. In some embodiments, the chelator is desferrioxamine (DFO) (N-(5-(3-((5-Aminopentyl)hydroxycarbamoyl)propionamido)pentyl)-3-((5-(N-hydroxyacetamido)pentyl)carbamoyl)propionohydroxamic acid), dodecane tetraacetic acid (DOTA) (2,2′,2″,2″-(1,4,7,10-tetraazacyclododecane-1,4,7,10-tetrayl)tetraacetic acid), 1,4,7-Triazacyclononane-1,4,7-triacetic acid (NOTA), [diethylenetriaminepentaacetic acid (DTPA) (N,N-Bis(2-(bis-(carboxymethyl)amino)ethyl)-glycine), or ethylenediaminetetraacetic acid (EDTA) 2-[2-[bis(carboxymethyl)amino]ethyl-(carboxymethyl)amino]acetic acid, desferrioxamine B (Df) (N1-(5-Aminopentyl)-N1-hydroxy-N4-(5-(N-hydroxy-4-((5-(N-hydroxyacetamido)pentyl)amino)-4-oxobutanamido)pentyl)succinamide), or succinylated-derivatives thereof. The chelator may be bonded to a free —COOH, —NH2, or —SH group on an amino acid residue side chain.

Additional radioactive isotope chelators are known in the art. See, for example, U.S. Pat. Nos. 4,479,930, 4,668,503, 4,986,979, 8,309,300, 8,436, 147, 8,968,741, 9,000,130, 9,486,537, and 10,517,945, and Vosjan et al., Nat. Protoc. 5(4):739-43 (2010), Calcagno and Fayad, Q. J. Nucl. Med. Mol. Imaging 64(1):74-84 (2020), Boughdad et al., J. Immunother. Cancer. 9(10):e003594 (2021), Boursier et al., Eur. J. Nucl. Med. Mol. Imaging 49(4):1433-1434 (2022), and Toner et al., Sci. Rep. 12(1):6185 (2022).

In some embodiments, one or more radioactive isotopes are connected indirectly to at least one of the VL, VH, and CH3 domains by one or more pre-labeled reagents, in which the one or more reagents are chemically modified with a radioactive isotope (e.g., iodinated) and purified, and then connected to at least one of the VL, VH, and CH3 domains.

In some embodiments, the radiolabeled tracer further contains a polyethylene glycol (PEG) molecule (i.e., it is PEGylated). PEG is a synthetic, hydrophilic, non-toxic, FDA-approved, non-immunogenic, polymer with widespread use in biomedical and other applications and may decrease non-specific kidney uptake and enhance image signal-to-noise ratios. See, e.g., Rashidian et al., J. Exp. Med. 214(8):2243-2255 (2017) and Teunissen et al., Star Protoc. 2(2):100434 (2021).

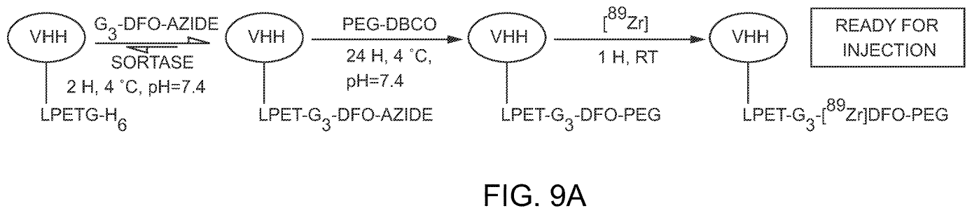

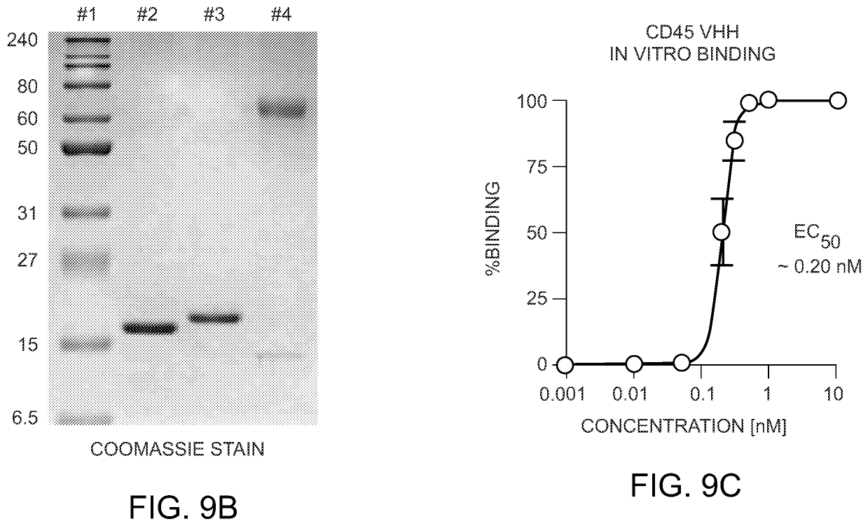

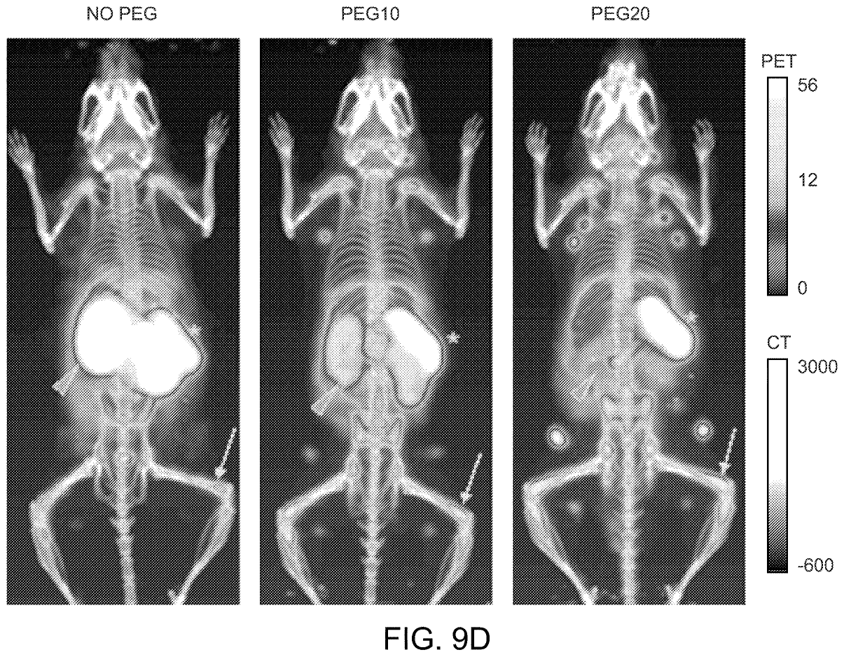

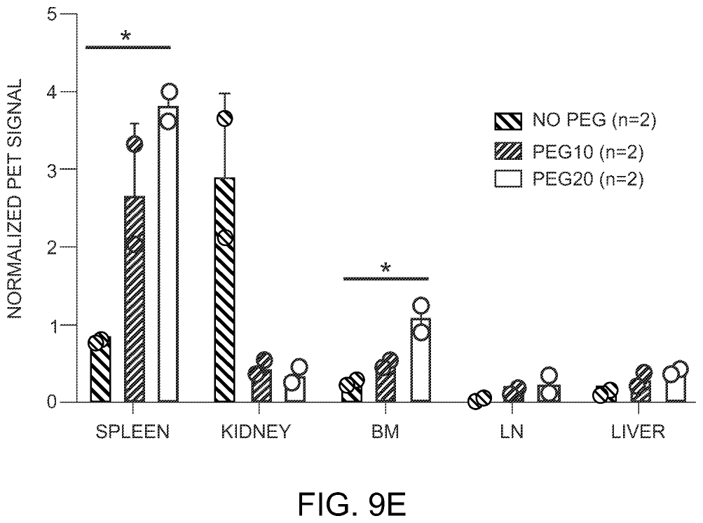

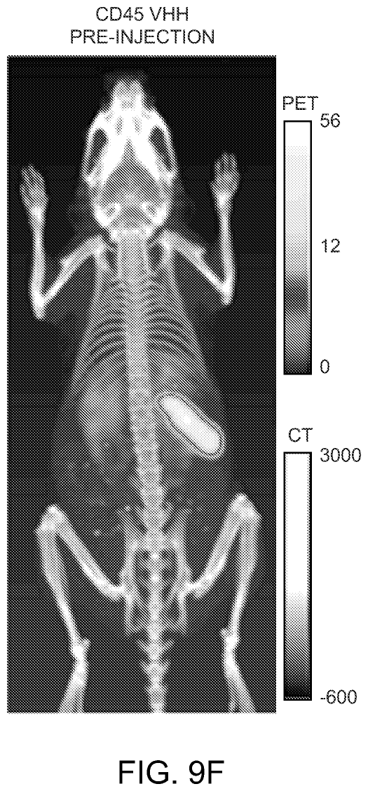

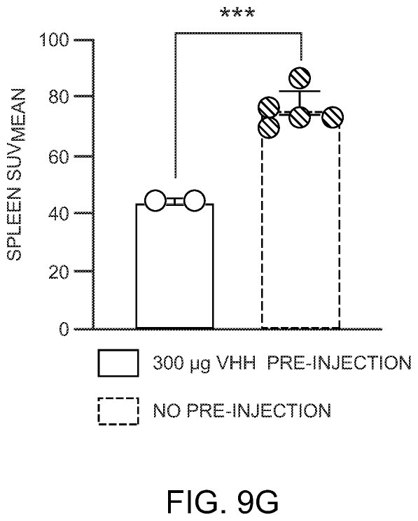

In some embodiments, the PEG molecule has a molecular weight of about 5 kilodaltons (kDa), about 10 kDa, about 15 kDa, or about 20 kDa. The PEG molecule may be connected to any moiety of the radiolabeled probe (i.e., at least one of the VL, VH, and CH3 domains) using standard laboratory techniques such as, for example, site-specific protein modification (e.g., with sortase) or azide-alkyne click chemistry (FIG. 9A) at, for example, the C terminus of the scFv.

In some embodiments, the radiolabeled tracer further contains a purification tag. A purification tag typically entails addition of in-frame nucleic acids that will be translated into amino acids along with the protein to which the tag is attached. The purification tag allows the protein to be captured or detected. Representative examples of purification tags include polyhistidine, FLAG epitope, histidine affinity tag (HAT), herpes simplex virus (HSV) epitope, human influenza hemagglutinin (HA), glutathione S-transferase (GST), KT3 epitope, maltose binding protein (MBP), AU1 epitope, AU5 epitope, Bacteriophage T7 epitope, myc tags. Amino acid sequences of representative purification tags are listed in Table 3.

TABLE 3
Amino acid sequences of representative
purification tags

| Purification Tag | Sequence |
|---|---|
| AU1 epitope (SEQ ID NO: 34) | DTYRYI |
| AU5 epitope (SEQ ID NO: 35) | TDFYLK |
| Polyhistidine (SEQ ID NO: 36) | HHHHHH |
| FLAG epitope (SEQ ID NO: 37) | DYKDDDK |
| FLAG His tag (SEQ ID NO: 38) | EFDYKDDDDKGS |
| | LPETGGHHHHHH |
| HAT (SEQ ID NO: 39) | KDHLIHNVHKEF |
| | HAHAHNK |
| HSV epitope (SEQ ID NO: 40) | QPELAPED |

The radiolabeled tracer may be present as a scFv-CH3 domain monomer which will form a minibody homodimer containing two scFv-CH3 domain monomers. The CH3 domains in each minibody monomer enable dimerization. The term “minibody” as used herein refers to an engineered antibody fragment that contains a VL domain, a VH domain, and a CH3 domain.

### Diagnostic Composition

In one aspect, the present disclosure provides a diagnostic composition which includes an effective amount of the radiolabeled tracer and a pharmaceutically acceptable carrier.

The effective amount of a radiolabeled tracer for a given patient varies depending one or more factors that may include the age, body weight, type, and general health of the subject. Ultimately, the attending physician will decide the appropriate dose and dosage regimen. Typically, the radiolabeled tracer will be given in a single dose before imaging of the subject. In some embodiments, the effective amount of the radiolabeled tracer is about 37 megabecquerel (MBq) (about 1 mCi) to about 148 MBq (about 4 mCi) per dose. In some embodiments, the effective amount of the radiolabeled tracer is about 0.1, about 0.5, about 1, about 2, about 4, about 5, about 6, about 8, about 10, or about 20 mg protein per dose. In some embodiments, the effective amount of the radiolabeled tracer is about 2 mg to about 8 mg protein per kg of subject weight per dose.

Diagnostic compositions may be provided as sterile lyophilized solid or liquid preparations. Lyophilized solid compositions may be prepared by freezing a liquid composition and removal of the frozen liquid by sublimation. Lyophilized solid preparations may be reconstituted and diluted into a liquid preparation before use. Pharmaceutically acceptable liquid carriers include aqueous or non-aqueous carriers alike. Representative examples of liquid carriers include sterile water for injection, saline, Lactated Ringer Injection solution, buffered saline, phosphate buffered saline, a soluble protein, soluble sugars (e.g., dextrose), dimethyl sulfoxide (DMSO), polyol (e.g., glycerol, propylene glycol, liquid polyethylene glycol, and the like), ethanol, and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Citric acid, sodium chloride, sugars, polyalcohols, and isotonic electrolyte solutions (e.g., Plasma-Lyte®) may be used to achieve the desired isotonicity. Depending on the carrier and the immunoctyokine, other excipients may be added, e.g., wetting, dispersing, or emulsifying agents, gelling and viscosity enhancing agents, preservatives and the like as known in the art. In some embodiments, the compositions include citric acid, ethylenediaminetetraacetic acid (EDTA), and polysorbate 20 with a pH range between about 6.8 to about 7.2.

### Methods of Imaging

In some aspects, the present disclosure is directed to detecting inflammation in a subject. The method entails administering intravenously to a subject in need thereof, a diagnostic composition containing an effective amount of the radiolabeled tracer and producing a first image of a tissue of the subject by PET or SPECT imaging, and wherein detecting the radioactive isotope in the tissue is an indication of inflammation in the tissue. Because the imaging methods detect immune cells, imaging methods may be used as an indicator of the severity of inflammation in the subject, or the effectiveness, or lack thereof, of therapy the subject is receiving for purposes of treating the inflammation as compared to an image of a normal, healthy control subject with normal levels of resident immune cells.

The term “inflammation” as used herein refers to the immune system's response to harmful stimuli, such as pathogens, damaged cells, toxic compounds, or irradiation, and is characterized by the influx of CD45⁺ immune cells into an inflamed tissue. Inflammation is part of the normal healing process. Normal inflammation characterized by the temporally restricted upregulation of inflammatory activity that occurs when a threat is present and that resolves once the threat has passed (i.e., acute). The failure of acute inflammation to resolve results in chronic inflammation and may contribute to tissue injury and disease.

The term “inflammatory disease” as used herein refers to a state or condition where an inflammatory response to a specific stimulus is not resolved when the stimulus is removed, and the inflammation continues to exert a deleterious effect on the cells, tissues, and/or organs of the body. The term “inflammatory disease” as used herein encompasses diseases that present with inflammation (e.g., possess an inflammatory component) alone, or with other non-inflammatory components, for example, cancer, diabetes mellitus, neurodegenerative conditions, ischemic heart disease, non-alcoholic fatty liver disease, and autoimmune conditions.

In some aspects, the present disclosure is directed to detecting the presence of CD45⁺ immune cells in a subject. The method entails administering intravenously to a subject in need thereof, a diagnostic composition containing an effective amount of the radiolabeled tracer and producing a first image of a tissue of the subject by PET or SPECT imaging, wherein detecting the radioactive isotope in the tissue is an indication of the presence of CD45⁺ immune cells in the tissue.

The term “subject” (or “patient”) as used herein includes all members of the animal kingdom prone (or disposed) to or suffering from the indicated inflammation. In some embodiments, the subject is a human. Therefore, a subject “having inflammation” or “in need of” imaging according to the present disclosure broadly embraces subjects who have been positively diagnosed with inflammation, including subjects having active inflammation who may have been previously treated with one or more rounds of therapy, and subjects who are not currently being treated (e.g., in remission) but who might still be at risk of additional inflammation, and subjects who have not been positively diagnosed but who are predisposed to having inflammation (e.g., on account of the basis of prior medical history and/or family medical history, or who otherwise present with a one or more risk factors such that a medical professional might reasonably suspect that the subject was predisposed to inflammation).

The term “tissue” as used herein refers to a group of cells and intercellular matrix that have similar structure and that function together as a unit. An image of a tissue embraces taking an image of the entire organ in which the tissue resides. For example, an image of cardiac tissue embraces taking an image of the heart.

In some embodiments, the inflammation detected in the imaged tissue has progressed to an inflammatory disease state. In other embodiments, the inflammation detected in the imaged tissue has not progressed to an inflammatory disease state. In some embodiments, the imaged inflammation detected in the imaged tissue has normal immune infiltration or resident immune cells. In some embodiments, the inflammation detected is focal (i.e., a portion of an organ).

In some embodiments, the subject has been previously diagnosed with coronavirus disease.

In some embodiments, the subject is suspected of having fever of unknown origin (FUO), which is defined as fever of 38.3° C. (101° F.) or higher for 3 or more weeks in immunocompetent, otherwise healthy patients with no identified cause of fever after undergoing a set of obligatory investigations. In some embodiments, the subject is suspected of having vasculitis.

In some embodiments, the subject is taking or will begin taking a therapy for an inflammatory disease. In some of these embodiments, the intensity of the signal in the imaged tissue may correlate with the effectiveness of the therapy. For example, the presence and intensity of signal in the imaged tissue may reflect an ineffective therapy. A low intensity signal or even absence of signal in the imaged tissue may reflect an effective therapy.

The intensity of signal in the imaged tissue may also be used as an indicator of the severity of the inflammation and of the inflammatory disease. The amount of signal that qualifies as above normal may be determined by a physician skilled in analyzing such images, and as described below in the Image analysis section.

Methods and techniques for analyzing images produced from PET, PET-CT, PET-MRI, or SPECT are known in the art. Areas or locations of elevated radiolabeled tracer (i.e., increased signal on the image) represent CD45⁺ immune cells bound to the radiolabeled tracer, thereby enabling the detection of these immune cells (e.g., immune cell infiltrates) in the image.

In some embodiments, the imaging comprises use of PET. PET scanners and techniques are known in the art. See, for example, U.S. Pat. Nos. 8,660,636, 9,495,771, 10,909,731, 11,069,098, and 11,300,695.

In some embodiments, the PET imaging is conducted via PET-CT. PET-CT scanners and techniques are known in the art. See, for example, U.S. Pat. Nos. 7,507,968, 7,569,829, 7,813,535, 7,869,860, 8,063,376, and 9,155,514.

In some embodiments, the PET imaging includes concomitant use of MRI (PET-MRI). Combined PET-MRI techniques are known in the art. See, for example, U.S. Pat. Nos. 4,939,464, 7,835,782, 7,888,632, 8,073,525, 8,073,527, 8,452,378, 8,467,845, 9,459,333, 9,510,797, 9,555,135, and 10,627,466, and Senders et al., J. Am. Coll. Cardiol. 71(3):321-335 (2018).

In some embodiments, the imaging comprises use of SPECT. SPECT scanners and techniques are known in the art. See, for example, U.S. Pat. Nos. 7,109,489, 7,635,848, 7,683,332, 7,968,851, 8,421,021, and 8,462,911.

Additional PET and SPECT image analysis techniques are known in the art. See, for example, U.S. Pat. Nos. 7,251,523, 7,876,938, 9,204,835, 9,846,922, 10,482,600, 10,943,681, 11,055,847, 11,164,045, 11,222,243, 11,301,995, and 11,424,035.

The present compositions and methods may detect inflammation in any bodily organ or tissue. In some embodiments, the imaged tissue is cardiac tissue, lung tissue, liver tissue, kidney tissue, digestive tissue, skin tissue, muscle tissue, immune tissue, nervous system tissue, or bladder tissue. In some embodiments, the skin tissue is the abdominal skin or nose skin. In some embodiments, the imaged tissue includes more than one tissue or organ. In some embodiments, the imaged digestive tissue includes the large bowel, the rectum, the anal canal, or a combination of two or more thereof. In some embodiments, the imaged cardiac tissue includes the heart. In some embodiments, the immune tissue is spleen, lymph nodes, bone marrow, or a combination of two or more thereof. In some embodiments, the imaged tissue includes from the base of the skull to the to mid-thigh. In some embodiments, the imaged tissue is a substantial portion of the whole body.

In some embodiments, the imaged tissue is cardiac tissue. In some of these embodiments, the imaged tissue is the myocardium, in others the imaged tissue is the heart. In some embodiments, the subject is suspected of having an inflammatory cardiomyopathy. In some embodiments, the tissue is the digestive tissue, and the subject is suspected of having an inflammatory bowel disease (IBD). In some of these embodiments, the imaged tissue is the large bowel, the rectum, the anal canal, or a combination of two or more thereof. In some embodiments, the imaged tissue is lung tissue, and the subject is suspected of having a pulmonary inflammatory disease. In some embodiments, the imaged tissue is at least one of skin tissue, liver tissue, digestive tissue (e.g., colon), kidney tissue, liver tissue, skin (e.g., abdominal skin, nose skin, and mouth skin), immune tissue (e.g., spleen and bone marrow), and lung tissue, and the subject is suspected of having graph-versus-host disease (GVHD). In some embodiments, the imaged tissue is a substantial portion of the whole body. In some embodiments, the substantial portion of the whole body includes the skin tissue, liver tissue, digestive tissue, and lung tissue. In some of these embodiments, the subject is suspected of having a FUO. In some of these embodiments, the subject is suspected of having vasculitis. A skilled attending physician would recognize the signs, symptoms, and preconditions in order to suspect a subject of having an inflammatory cardiomyopathy, an IBD, a pulmonary inflammatory disease, GVHD, FUO, or vasculitis.

The image of the subject's tissue is taken after administration of the radiolabeled tracer. In some embodiments, the image is taken about 2 hours to about 96 hours after administering the radiolabeled tracer. In some embodiments, the image is taken about 2 hours to about 24 hours after administering the radiolabeled tracer. In some embodiments, the image is taken about 2 hours to about 5 hours after administering the radiolabeled tracer. In some embodiments, the image is taken on the same calendar day as the radiolabeled tracer is administered.

### Image Analysis

CD45⁺ immune cell infiltrates are immune cells present in inflamed tissue that are not normally present in healthy, non-inflamed tissue, or exist in low quantities (representing resident immune cells), and therefore serve as an indication of inflammation.

In some embodiments, the method of imaging inflammation in a subject entails identifying a first location of elevated radiolabeled tracer amount within the image relative to a control image of a non-inflamed tissue. The first location of elevated radiolabeled tracer may be manually identified, commonly referred to as a region of interest (ROI) analysis or by automated methods, commonly referred to as a voxel-based analysis. See, Snook et al., Neuroimage 34(1):243-52 (2007).

The ROI analysis method involves manually defining an area of elevated radiolabeled tracer in the image within which to make measurements (i.e., quantify the amount of radioactive isotope within the image or location). The voxel-based analysis method involves a voxel-by-voxel statistical comparisons throughout the image. The voxel-based analysis method spatially normalizes each set of images to an image of a healthy, non-inflamed tissue, and thus assigning an “address” to each voxel. Then, a voxel-by-voxel statistical comparison of the images can be performed. This voxel-by-voxel statistical comparison can be used to quantify the amount of radioactive isotope within the image or location.

Radiolabeled tracers used in PET and SPECT emit γ radiation, which is detected by a PET or SPECT detector, as known in the art. The resulting data on the emitted radiation collected by the detector is converted into voxel data to form an image. Methods for converting detector data into voxel data are known in the art, for example, by the filtered back projection method. A voxel represents a value on a regular grid in three-dimensional space. Each voxel in the image records the intensity of γ radiation received by the detector.

The image may be evaluated, for example, by a nuclear radiologist, to determine if the radiolabeled tracer is present in the imaged tissue. A level of radioisotope greater than the background present in healthy, non-inflamed tissue may be indicative of inflammation in the imaged tissue. See, for example, Calcagno and Fayad, Q. J. Nucl. Med. Mol. Imaging 64(1):74-84 (2020).

The image may be evaluated or quantified as the percent injected dose (% ID), which is the amount of activity present in a region, organ, or image as a percentage of the total available activity or dose. Dividing the % ID by the injected subject's body weight produces % ID per gram (% ID/g) (Behr et al., Hybridoma 16(1):101-107 (1997)). The image may also be evaluated or quantified with the standardized uptake value (SUV), which is the ratio of tissue radioactivity concentration at a given time and the administered dose at the time of injection divided by body weight (Tahari et al., J. Nucl. Med. 55(9):1481-1484 (2014); Verbraecken et al., Metabolism 55(4):515-524).

A lack of signal (e.g., signal no greater than background) in the imaged tissue would indicate a non-inflammatory state, in which case the attending physician may advise additional tests for conditions other than inflammation. For example, the lack of radiolabeled tracer in cardiac tissue may indicate that the subject has a condition other than inflammatory cardiomyopathy.

### Longitudinal Studies

Repeated imaging of the same subject over a period of time, referred to as a longitudinal study, can be used to monitor the progression of a disease or evaluate the effectiveness of a treatment. The methods disclosed herein broadly embrace longitudinal studies involving one or more subsequent administrations of the radiolabeled tracer and imaging of the subject (for a total of two or more administration and imaging events).

In some embodiments, the method further entails administration of a second effective amount of the radiolabeled tracer after the first image is produced, producing a second image of the tissue. In some embodiments, the method entails identifying a first location of elevated radiolabeled tracer within the second image. The time period between the initial and subsequent administration of the radiolabeled tracer may be determined by the attending physician. In some embodiments, the second image is produced at least two weeks after the first image was produced.

The image analysis may involve quantifying the amount of the radiolabeled tracer in the second image. The image analysis may also involve comparing the images produced after the two radiolabeled tracer administrations. The images may be compared as a whole, or specific locations of elevated radiolabeled tracer may be compared between images.

### Inflammatory Diseases

In some aspects, the present disclosure is directed to treating an inflammatory disease in a subject. The method entails imaging a tissue of a subject in need thereof by administering intravenously to the subject a diagnostic composition containing the radiolabeled tracer, producing a first image of a tissue of the subject by PET or SPECT imaging, wherein detecting the radioactive isotope in the tissue is an indication of inflammation, and if the radiolabeled tracer is detected, administering an appropriate therapy to the subject.

The terms “treat”, “treating”, and “treatment” as used herein refer to any type of intervention, process performed on, or the administration of an active agent to the subject in need thereof (e.g., a subject suspected of having inflammation or who has been diagnosed with inflammation and who may be undergoing treatment) with the therapeutic objective (“therapeutic effect”) of reversing, alleviating, ameliorating, inhibiting, diminishing, slowing down, arresting, stabilizing, or preventing or delaying the onset, progression, development, severity or recurrence of a symptom, complication or condition, or biochemical indicia associated with an inflammatory cardiomyopathy.

In some embodiments, inflammatory disease is an inflammatory cardiomyopathy, an inflammatory bowel disease (IBD), a pulmonary inflammatory disease, or graph-versus-host disease.

In some embodiments, the tissue is cardiac tissue, and the inflammatory cardiomyopathy is myocarditis, cardiac sarcoidosis, or cellular cardiac transplant rejection. See, e.g., Giblin et al., Card. Fail. Rev. 7:e17-10 (2021), Martineau et al., Am. J. Nucl. Med. Mol. Imaging 11(1):46-58 (2021), Park et al., J. Nucl. Cardiol. 29(5):2171-2187 (2022), and Toner et al., Sci. Rep. 12(1):6185 (2022).

In some embodiments, the tissue is digestive tissue (e.g., large bowel, rectum, or both the large bowel and the rectum), and the IBD is eosinophilic esophagitis, chronic gastroesophageal reflux, Helicobacter pylori infection, Crohn's disease, or ulcerative colitis.

In some embodiments, the tissue is lung tissue, and the pulmonary inflammatory disease is acute respiratory distress syndrome (ARDS), chronic obstructive pulmonary disease (COPD), pneumonia, or an interstitial lung disease. Representative interstitial lung disease that may be imaged include atypical pneumonia, idiopathic pulmonary fibrosis, sarcoidosis, bronchiolitis obliterans, hypersensitivity pneumonitis, scleroderma, asbestosis, eosinophilic pneumonias, cryptogenic organizing pneumonia, pulmonary alveolar proteinosis, dermatomyositis, mixed connective tissue disease, rheumatoid arthritis, vasculitis, histiocytosis x, interstitial lung and liver disease, lupus, pulmonary hypertension, respiratory failure, and silicosis. See, e.g., Pauwels et al., Am J Respir Crit Care Med. 163(5):1256-1276 (2001), Cottin, Eur. Respir. J. 48(6):1274-1277 (2016), and Khadangi et al., Sci. Rep. 11(1):7777, pp. 1-12 (2021).

In some embodiments, the tissue is at least one of skin tissue, liver tissue, digestive tissue, and lung tissue, and the inflammatory disease is graph-versus-host disease. See, e.g., Ferrara et al., Lancet 373(9674):1550-1561 (2009), Aslanian et al., Dig. Dis. Sci. 57(3):720-725 (2012), Khoury et al., Haematologica 102(5):958-966 (2017), and Malard et al., Nat. Rev. Dis. Primers 9(1):27 (2023).

In some embodiments, the anti-inflammatory treatment includes administration of one or more anti-inflammatory agents if the radiolabeled tracer is detected in the imaged tissue. The dosage of the anti-inflammatory agent may be the same as known or recommended doses without the inventive imaging. See, Hardman et al., eds., Goodman & Gilman's The Pharmacological Basis of Therapeutics, 10th ed., McGraw-Hill, New York, 2001; Physician's Desk Reference 60th ed., 2006.

In some embodiments, two or more additional anti-inflammatory agents are used in combination and are cyclically administered. Cycling therapy involves the administration of one anti-inflammatory agent for a period of time, followed by the administration of a second active anti-inflammatory for a period of time and repeating this sequential administration, i.e., the cycle, in order to reduce the development of resistance to one or both of the anti-inflammatory agents, to avoid or reduce the side effects of one or both of the anti-inflammatory agents, and/or to improve the efficacy of the anti-inflammatory agents. In one example, cycling therapy involves the administration of a first anti-inflammatory agent for a period of time, followed by the administration of a second anti-inflammatory agent for a period of time, optionally, followed by the administration of a third anti-inflammatory agent for a period of time and so forth, and repeating this sequential administration, i.e., the cycle in order to reduce the development of resistance to one of the anti-inflammatory agents, to avoid or reduce the side effects of one of the therapeutics, and/or to improve the efficacy of the anti-inflammatory agents.

In some embodiments, the anti-inflammatory agent is an angiotensin-converting enzyme (ACE) inhibitor, for example, captopril (Capoten®), enalapril (Vasotec®) fosinopril (Monopril®), linsopril (Prinivil®, Zesrtril®), perindopril (Aceon®), quinapril (Accupril®), rampiril (Altace®), lisinopril, or trandolapril (Mavik®) or an angiotensin II receptor blocker (e.g., candesartan (Atacand®), losartan (Cozaar®), or valsartan (Diovan®)). In some embodiments, the anti-inflammatory agent is sacubitril, valsartan, ivabraine (Corlanor®), spironolactone (CaroSpir®, Aldactone®), ivabradine, or a cardiac glycoside. In some embodiments, the anti-inflammatory agent is a nonsteroidal anti-inflammatory drug (NSAID) (e.g., ibuprofen, or indomethacin) or a renin-angiotensin system blockader. In some embodiments, the anti-inflammatory agent is a mineralocorticoid receptor antagonist (antimineralocorticoid), for example, spironolactone (Aldactone®, Spirix®, Spiron®), eplerenone (Inspra®), canrenone (Contaren®, Luvion®, Phanurane®, Spiroletan®), finerenone, or mexrenone. In some embodiments, the anti-inflammatory agent is an immunosuppressive drug, for example, azathioprine (Azasan®), prednisone (Prednisone Intensol®, Deltasone®, Rayos®) or cyclosporine (Sandimmune®, Neoral®, Gengraf®).

In some embodiments, the anti-inflammatory agent is beta blocker, for example, bisoprolol (Zebeta®), metoprolol succinate (Toprol XL®), carvedilol (Coreg®), or carvedilol CR (Coreg CR®). In some embodiments, the anti-inflammatory agent is an aldosterone antagonist, for example, spironolactone (Aldactone®) or eplerenone (Inspara®). In some examples, the anti-inflammatory agent is hydralazine combined with isoorbide dinitrate (Bidil®), diuretics (e.g., furosemide (Lasix®), bumetanide (Bumex®), torsemide (Demadex®), chlorothiazide (Diuril®), hydrochlorothiazide (Esidrix®, Hydrodiuril®), indapamide (Lozol®), metolazone (Zaroxolyn®), or triamterene (Dyrenium®).

In some embodiments, the anti-inflammatory agent is an anticoagulant, statin, digoxin, or intravenous immunoglobulin, In some embodiments, the anti-inflammatory agent is a corticosteroid, for example, triamcinolone (e.g., Kenalog®, Nasacort®, Adcortyl®, Aristocort®), prednisolone (Cotolone®, Prelone®, Pediapred®, Omnipred®, Pred®, Bubbli-Pred®), betamethasone (Celestone®), cortisone acetate, dexamethasone (Decadron®, Dexamethasone Intensol®), prednisone (Deltasone®), deflazacort (Emflaza®), budesonide (Entocort®), fludrocortisone (Florinef®), methylprednisolone (Medrol®), or methylprednisolone (Methylpred®).

In some embodiments, the anti-inflammatory agent is one or more anti-rejection medications, for example, tacrolimus (Prograf®) cyclosporine (Neoral®, Gengraf®), prednisone, mycophenolate (CellCept®, Myfortic®), azathioprine (Imuran®), anti-thymocyte globulin (Thymoglobulin®), steroids, tacrolimus (Protopic®, Envarsus XR®, Astagraf XL®), sirolimus (Rapamune®), everolimus (Zortress®), mycophenolate mofetil (CellCept®), daclizumab, or muromonab-CD3 (Orthoclone OKT3®).

In some embodiments, the anti-inflammatory agent is one or more anti-microbial agents. In some embodiments, the anti-microbial agent is an antibacterial agent, an antifungal agent, an antiviral agent, or a combination of two or more thereof. In some embodiments, the antibacterial agent is ciprofloxacin (Cipro®) and metronidazole (Flagyl®).

In some embodiments, the anti-inflammatory agent is one or more anti-diarrheal medications, for example, the anti-diarrheal agent is psyllium (Metamucil®), methylcellulose (Citrucel®), diphenoxylate and atropine sulfate (Lomotil®), and loperamide (Imodium A-D®).

### Kits

Diagnostic compositions may be assembled into kits or diagnostic systems for use in imaging inflammation or in treating an inflammatory cardiomyopathy. The kits or diagnostic systems may include one or more dosage formulations containing an effective amount of the radiolabeled tracer disposed in a suitable container, e.g., tube, vial, ampoule, bottle, syringe, or bag and printed instructions for use in intravenously administering the composition to a subject having or suspected of having inflammation or having an inflammatory disease or condition. In some embodiments, the subject is suspected of having inflammation, and wherein detection of the radioactive isotope is the subject may be indicative of an inflammatory disease or condition. In some embodiments, the radiolabeled tracer is present in the suitable container as formulated in a pharmaceutically acceptable liquid carrier. In some embodiments, the radiolabeled tracer is present in the suitable container as formulated as a lyophilized solid. In some embodiments, the kit includes a suitable container containing a pharmaceutically acceptable liquid carrier. The kits or diagnostic systems may also include one or more dosage formulations of an appropriate therapy for the treatment of an inflammatory cardiomyopathy, and a pharmaceutically acceptable carrier disposed in a suitable carrier. The kits or diagnostic systems of the disclosure may also comprise printed instructions for using the components therein.

These and other aspects of the present application will be further appreciated upon consideration of the following Examples, which are intended to illustrate certain embodiments of the application but are not intended to limit its scope, as defined by the claims.

### EXAMPLES

### Example 1: Materials and Methods

Humanization of NSG mice. NSG mice were injected with 1×10⁶ human peripheral blood mononuclear cells (PBMCs). The human immune cells will home to the lymphoid organs in the NSG mice. The mice were subjected to PET imaging one to two weeks after the infusion of the PBMCs.

Design and expression of the anti-murine CD45 nanobody. The CD45 nanobody (VHH) sequence (Rossotti et al., Biochim. Biophys. Acta. 1850(7):1397-1404 (2015)) was engineered with a C-terminal FLAG-tag (DYKDDDDK (SEQ ID NO: 37)), a sortase recognition motif (LPETG (SEQ ID NO: 41), and a His6-tag. It was then cloned into the pHEN6 vector (GenScript). The nanobody was expressed in the WK6 E. coli strain through periplasmic expression, following established protocols. In brief, the starter culture was diluted in 2 liters of terrific broth (TB) media with 2 mL ampicillin (100 μg/mL), and the OD600 was adjusted to 0.1. Upon reaching an OD600 of 0.5-0.8 after incubation at 37° C., isopropyl-beta-D-1-thiogalactopyranoside (IPTG) (1 mM) was added to induce nanobody expression and maintained overnight at 20° C. Following expression, cells were harvested by centrifugation at 12,000×g for 5 minutes. The resulting pellet was resuspended in 30 mL TES (0.2 M Tris, 600 μM EDTA, 0.5 M sucrose, pH 8) buffer and incubated at 4° C. for 2 hours. Subsequently, Tris buffer (50 mM Tris, pH 8, 4-fold dilution) was added to lyse the cells through osmotic shock. Centrifugation at 14,000×g for 15 minutes was performed to separate the supernatant. The nanobody was then purified from the supernatant using Nickel-NTA beads.

DFO-labeling and PEGylation of anti-mouse CD45 nanobody (VHH). The nanobody was site-specifically labeled with deferoxamine (DFO) and polyethylene glycol (PEG) in accordance with the established protocol (Rashidian et al., J. Exp. Med. 214(8):2243-2255 (2017)). Briefly, to prepare for DFO labeling, 1 L of 50 mM HEPES buffer containing 150 mM NaCl (pH 7.5) was treated with 10 g of Chelex® 100 sodium form beads at 4° C. for 16 hours to remove trace metals. Following Chelex® treatment, the protein, and all reagents, including sortase, underwent dialysis in metal-free HEPES buffer using Thermo Scientific™ SnakeSkin™ Dialysis Tubing for an additional 16 hours. The hepta mutant sortase A, which functions independently of Calcium ions, was employed (Jeong et al., Plos One 12(12):e0189068 (2017)). Reaction mixtures of 1 mL were composed of 50 mM HEPES (pH 7.5), 150 mM NaCl, 1.5 mM triglycine-containing DFO, 200 μM sortase-containing motif (i.e., substrate), and 5 μM sortase. Following overnight incubation at 4° C. with agitation, the reaction products underwent analysis through SDS-PAGE in a gel shift assay, with yields typically exceeding >95% DFO-labeled VHH. In instances where the yield fell below 80%, the reaction was extended for an additional 5 hours, accompanied by an increase in sortase concentration to 10 μM and triglycine-containing probe to 2 mM.

The labeled nanobody was subsequently purified using a desalting PD-10 size-exclusion column (GE Healthcare) with chelex-treated 50 mM HEPES buffer containing 150 mM NaCl (pH 7.5). Ni-NTA beads were introduced for further purification, and centrifugation was employed to eliminate sortase, which contains a His-6 tag, and any remaining unreacted His-tagged nanobody. The His-6 tag was engineered to be positioned downstream of the sortase tag, with the intention that, following the completion of sortase labeling procedure, the His-6 tag would be removed from the final product (FIG. 9A). The labeled nanobody was stored at −80° C. PEGylation was performed using DBCO-PEG molecules from ClickChemistryTools™ at a 2:1 molar ratio to the azide-DFO-labeled nanobody, and the reaction was allowed to proceed for 12 hours. PEGylation success was confirmed through SDS-PAGE characterization, typically yielding quantitative results.

Development of the anti-human CD45 antibody fragment. The DNA encoding the fusion of VH and VL (clone BC8) with the CH3 region of IgG1, optimized for HEK293 system expression, was cloned into a vector expression system featuring a signal sequence, enabling secretion of the protein into the supernatant (GenScript). A C-terminal FLAG-tag (DYKDDDDK (SEQ ID NO: 37)) and a His-6 tag were included in the design to allow characterization and purification, respectively. A stable HEK293 cell line was developed for enhanced production. To achieve this, HEK293 cells were transfected with pPAX2, pVSVG (packaging vectors), and the lentivirus plasmid containing the anti-human CD45 probe plasmid. The virus was collected at 48, 72, and 96 hours, sedimented at 20,000×g for 2 hours, and resuspended in Opti-MEM media. Subsequently, a new batch of HEK293 cells underwent three rounds of transduction with the virus. After recovery in DMEM complete media, puromycin selection was applied to retain cells integrating the lentivirus plasmid.

The selected cells were expanded in multiple culture dishes, washed with PBS, and incubated in serum-free DMEM for 48 to 72 hours. Harvesting the supernatant confirmed protein expression through SDS-PAGE analysis. Protein purification was performed via Ni-NTA metal affinity column, with non-specifically bound proteins removed via washing with a low imidazole concentration (20 mM). Subsequently, the imidazole concentration was increased to 300 mM to facilitate protein recovery. Further purification was achieved through size-exclusion chromatography, and the purified proteins were stored in 50 mM HEPES buffer at pH 7.5, at −80° C. until utilization.

Radiolabeling of the probe. The CD45 probe was labeled with p-SCN-Bn-NOTA following an established protocol (Vosjan et al., Nat. Protoc. 5(4):739-743 (2010)). The labeled NOTA-CD45 probe was purified via a PD-10 desalting column and was then used for radiolabeling. A 1.5 mL centrifuge tube was loaded with NOTA-labeled CD45 probe (300 μL, ~50 μM in PBS buffer) and ⁶⁴CuCl₂ (~1-2 mCi) was added to the reaction. The reaction was allowed to proceed at 37° C. for 30 min. Next, the mixture was loaded onto a PD-10 size-exclusion cartridge and eluted with PBS to provide the radiolabeled probe. The radiolabeling yield was typically above ~70%. Animals were injected with ~50 uCi of the CD45 probe and subjected to PET-CT imaging ~24 h later.

Probe binding assay. To assess the binding efficiency of the CD45 probes to murine and human immune cells in vitro, 1×10⁵ cells were incubated with varying concentrations (ranging from 0 nM to 20 nM) of their respective probes, which was either conjugated to DFO with or without non-radioactive Zirconium (Zr) ion (ZrCl₄). The binding of probes coupled to DFO and cold Zr was determined using anti-FLAG tag antibody PE (BioLegend #637309). Triplicate analyses were conducted for all probe concentrations. Subsequent analyses were conducted using a Sony SP6800 Spectral Analyzer, with data analysis performed using FlowJo v10.10 software.

DFO-labeling of the anti-human CD45 minibody probe was conducted using a previously established protocol.⁵² Briefly, to prepare for DFO labeling, 1 L of 50 mM HEPES buffer containing 150 mM NaCl (pH 7.5) underwent treatment with 10 g of Chelex® 100 sodium form beads at 4° C. for 16 hours to remove trace metals. After chelex treatment, the protein and 2 M Na₂CO₃ underwent dialysis in metal-free HEPES buffer using Thermo Scientific™ SnakeSkin™ Dialysis Tubing for an additional 16 hours. The pH of the probe solution was adjusted to pH 9 with 2 M Na₂CO₃.

Reaction mixtures of 1 mL were composed of 50 mM HEPES (pH 7.5), 150 mM NaCl, 1.5 mM p-isothiocyanatobenzyl-DFO, and 200 μM of the anti-CD45 minibody. Following 1 hour of incubation at room temperature and 12 hours of incubation at 4° C. with agitation, the DFO-labeled minibody was purified using a desalting PD-10 size-exclusion column (GE Healthcare) with chelex-treated 50 mM HEPES buffer containing 150 mM NaCl (pH 7.5). Finally, the buffer was exchanged to adjust the pH of the product to 7.5 through an 8-hour dialysis in metal-free HEPES buffer using Thermo Scientific™ SnakeSkin™ Dialysis Tubing at 4° C. The labeled minibody was stored at −80° C. until use.

Preparing ⁸⁹Zr-labeled CD45 probes. In a typical radiolabeling reaction, a solution was prepared by combining ~50-100 μg of chelexed DFO labeled CD45 nanobody or CD45 minibody with 200 μl of 50 mM HEPES buffer, pH 7.5. ⁸⁹Zr was obtained from Washington University in St. Louis (Mallinckrodt Institute of Radiology). Subsequently, a volume of the ⁸⁹Zr⁴⁺ stock solution, typically provided in 1.0 M oxalic acid and corresponding to 1.0 to 1.5 mCi, was added to a 2 mL plastic screw-cap microcentrifuge tube. The total volume of this solution was adjusted to 300 μL using 1.0 M oxalic acid. The pH of the ⁸⁹Zr⁴⁺ solution was set to 6.8-7.5 via a 2.0 M Na₂CO₃ solution, and this mixture was added to the CD45 probe solution.

The reaction mixture underwent a 1-hour incubation at room temperature on an agitating block at 350 rpm. Next, the radiolabeled probe was purified by a PD-10 size-exclusion cartridge (GE Healthcare) and elution with 1×PBS. The radiolabeling yield typically exceeded 70% (~1 mCi) of the ⁸⁹Zr-CD45 probe, as determined through decay-corrected radiochemical analysis.

Mouse studies. All mouse studies adhered to ethical and safety protocols and were approved by the Dana-Farber Cancer Institute Committee on Animal Care (protocol number 20-006) and the Boston Children's Hospital Committee on Animal Care (protocol number BCH 00001455). Mice were provided food and water ad libitum unless otherwise stated.

PET/CT imaging and analysis. Detailed PET/CT procedures have been previously outlined.⁷ For imaging experiments, mice were anesthetized with 1.5% isoflurane in O₂ at a flow rate of approximately 1 liter per minute. PET/CT imaging was conducted with a G8 PET/CT small-animal scanner (PerkinElmer). The G8 PET/CT has a peak sensitivity exceeding 14% of positron emission, along with a mean resolution of 1.4 mm. Each PET acquisition spanned 10 minutes, followed by a 2-minute CT scan. After in vivo imaging completion, anesthetized mice underwent trans-cardiac perfusion with 20 mL PBS, and organs of interest were collected for downstream ex vivo PET/CT experiments (10-minute PET and 2-minute CT scans).

Image processing utilized the manufacturer's automatic image reconstruction software, with subsequent data analysis and quantification performed using VivoQuant software (2022, In Vicro Solutions). PET signal intensity was measured as μCi/mm³. CT scans were overlaid with PET signal to guide the generation of 3D regions of interest (ROIs), which represented a certain organ within the mouse. To account for the variation in immune infiltration in the inflammatory models, areas of high signal in organs of interest were compared across each mouse by determining the PET signal minima and maxima for each organ of interest ROI. Subsequently, a threshold was generated at the 75% of the maximum PET intensity in that ROI. A second ROI was generated, encompassing only those voxels reaching above threshold. The mean standard uptake value (SUVₘₑₐₙ) was calculated for this ROI using the mouse's weight and radioactivity at the time of imaging: SUV=concentration in ROI/(injected dose/mouse body weight). To account for background signal differences, as in the case with the PEG optimization experiment, the SUVₘₑₐₙ of organs of interest was normalized against that of muscle, which should have nearly no CD45-specific uptake. These results are presented as a unitless ratio. Quantification of radioactivity was decay corrected.

Acute respiratory distress syndrome (ARDS) model. In the ARDS imaging studies, a previously published protocol was used (Khadangi et al., Sci. Rep. 11(1):7777, pp. 1-12 (2021)). Briefly, 5- to 6-week-old C57BL/6 mice (purchased from Jackson Laboratory) were weighed and then anesthetized with isoflurane and positioned supine with elevated heads. Lipopolysaccharide (LPS) suspended in saline (prepared as either 3 μg/μL or 1 μg/μL of saline) was administered intranasally at a dose of 3 μg/g (high dose) or 1 μg/g (low dose) of mouse body weight, dispensed slowly into the left nostril across 2-3 tidal breaths. Twenty-five (25) μL saline were administered to the control mice using the same protocol. Six hours post-LPS administration, the mice were injected with the ⁸⁹Zr-labeled-CD45-VHH-PEG20 PET probe (referred to as the “CD45-PET probe”) and subsequently underwent PET/CT imaging 24 hours after the probe administration. Following LPS administration, animals were closely monitored for weight changes.

Inflammatory bowel disease (IBD) colitis model. A well-established dextran sodium sulfate (DSS)-induced colitis model was utilized, where 5- to 6-week-old C57BL/6 mice were given autoclaved drinking water supplemented with DSS (36-50 kDa MW, MP Biomedicals, #160110) as a percentage of weight per volume of water. Mice with regular water regimen were used as control. DSS-colitis disease activity index (DAI) was evaluated every other day. The DAI is the combined score of weight loss compared to baseline weight, stool consistency, and bleeding and is used to evaluate clinical progression of colitis. The scores are assigned according to the following criteria: no weight loss (0); 1-5% weight loss (1); 5-10% weight loss (2); 10-20% weight loss (3); >20% weight loss (4); normal stool consistency (0); loose stool (2) and diarrhea (4); no blood (0); visual pellet bleeding (2); and gross bleeding with blood around anus (4) (Kim et al., J. Vis. Exp. (60):3678, pp. 1-6 (2012)). On the last day of the DSS exposure period, mice were i.v. injected with the CD45-PET probe and underwent CD45-PET imaging 24 hours later.

Experimental acute colitis cohorts included mice exposed to 4% (w/v) DSS over 5 or 7 days. The colitis recovery model was induced by administering 2% DSS over 8 days, at which point mice underwent their first CD45-PET imaging timepoint. Mice were then randomized to one of two 7-day recovery regimens: either regular water alone or regular water coupled with the intraperitoneal injection of dexamethasone at a dose of 1 mg/kg for three days following their first timepoint of imaging. Mice were then re-imaged with CD45-PET at day 8 of recovery. In all studies, mice were euthanized following the final imaging session, and tissues were collected for ex vivo imaging, gamma radiation assays and histological evaluation.

¹⁸F-FDG-PET versus CD45-PET imaging comparison in acute colitis. Following 7 days of 4% DSS exposure, acute colitis mice underwent both ¹⁸F-FDG-PET versus CD45-PET imaging to allow for direct performance comparison. On the final day of exposure (day 7), mice were intravenously injected with 80 μCi of ¹⁸F-FDG. Approximately 90 minutes later, mice underwent PET/CT with each PET acquisition spanning 10 minutes, followed by a 2-minute CT scan. Mice were then injected with the CD45-PET probe and underwent CD45-PET imaging 24 hours after probe injection.

Humanization and graft-versus-host disease. The experiments utilized 5- to 6-week-old NOD-scid H2-K1null H2-Ab1null H2-D1null IL2Rgnull mice (NSG-MHC I/II double knockout (NSG-dKO)), purchased from Jackson Laboratory. For humanization, the mice were intravenously injected with 10 million human PBMCs. Non-humanized NSG-dKO were used as controls. PET/CT imaging was conducted using the human CD45-PET probe three weeks post-humanization, at which point engraftment was expected. A portion of humanized mice and non-humanized controls were sacked for ex vivo imaging and biodistribution. Remaining mice were then monitored for the development of graft-versus-host disease (GVHD) symptoms using published criteria (Naserian et al., Front. Immunol. 9:10, pp. 1-9 (2018)). In brief, mice were given 1 point for each of the following symptoms: weight loss, hunched posture, dull fur texture, skin lesions, and diarrhea. As previously described, mice with scores of 0-1 had low or no GVHD, 2-3 had mild GVHD, and 4-5 had severe GVHD. Once most mice (4/5) developed 1 or more GVHD symptom, mice underwent a second round of CD45-PET imaging approximately 4 months after humanization. Subsequently, the animals were euthanized, and organs were collected for further ex vivo, gamma counting, and histological assessments, as elaborated in the main manuscript.

Gamma radiation assay. Following the completion of in vivo and ex vivo PET/CT imaging, the specific radioactivity of individual organs was measured using a Perkin-Elmer Wallac Wizard 3″ 1480 Automatic Gamma Counter as a confirmatory assay. Weights were recovered for each organ. Counting efficiency was calculated as 78%, and all counts were decay- and background-corrected. CD45-PET probe uptake was expressed as the percent of injected dose per gram of tissue (% ID/g).

H&E and IHC staining. Organs intended for histologic assessment were fixed for at least 24 hours in 10% buffered formalin before being transferred to 70% ethanol. All subsequent procedures were carried out by the Dana-Farber Cancer Institute Histology Core facility, employing established laboratory techniques. In brief, organs were embedded in paraffin, cut transversally in 5 μm-thick sections, and were either stained with hematoxylin and eosin (H&E) or underwent Immunohistochemistry (IHC) targeting for murine CD45 or human CD45 following deparaffinization, antigen retrieval and endogenous enzyme blocking.

Quantification of murine CD45⁺ staining in ARDS lung samples. To assess whether lung PET signal correlated with immune infiltration, lung samples from mice induced with ARDS underwent IHC for CD45⁺ immune cells. For each mouse, stained lung samples were initially scanned the stained slides with a Nikon ECLIPSE Ti2 Microscope at 4× magnification. After assessing the entire tissue sample on the slide, 3 representative sections of the tissue were then chosen, which had the highest level of IHC staining, and captured non-overlapping images of these sections at 10× magnification. Two independent and blinded investigators quantified the images using the following procedure. Using ImageJ image processing software, a threshold was applied to subtract background from the field of view. Investigators then quantified the percentage of positively stained pixels of each 10× image, which was then averaged with quantifications of all 3 10× sections to obtain an overall value for each mouse.

Histologic inflammation scoring of colitis bowel samples. To facilitate comparison across mice, the distal colon from each mouse was excised from the rest of the colon and flushed with PBS in preparation for H&E staining and histologic assessment. The severity of inflammation was evaluated by two blinded investigators. For each mouse, 3 random non-overlapping images were taken of a representative tissue section. Inflammation severity was scored using previously published criteria: no inflammatory infiltrate was graded as normal (0), focally small or widely separated multifocal areas of inflammation limited to the lamina propria were graded as mild (1), multifocal or locally extensive areas of inflammation extending to the submucosa were graded as moderate (2), inflammation extended into all layers of the intestinal wall or the entire intestinal epithelium was destroyed, lesions were graded as severe (3) (Chassaing et al., Curr. Protoc. Immunol. 104:15, pp. 1-16 (2014)) The scores for each of the 3 sections were averaged to obtain an overall value for each mouse. This was then correlated with the ex vivo SUV of the distal colon segment.

Statistical analysis. All statistical analyses were conducted using GraphPad Prism 10 (GraphPad Software Inc.). The specific statistical tests performed for each analysis are outlined in the figure legends. The following p-values were used: p>0.05 (not significant), p≤0.05 (*), ≤0.01 (**), ≤0.001 (***), ≤0.0001 (****).

### Example 2: Cardiac Inflammation Specific Anti-CD45 Immuno-PET

In one embodiment, the radiolabeled tracer is a minibody that binds CD45. An antibody fragment that binds CD45 was derived from the antibody clone BC8. A single-chain antibody fragment (scFv) from BC8 was attached to a CH3 domain, which facilitates dimerization, and which has the amino acid sequence set forth in SEQ ID NO: 33.

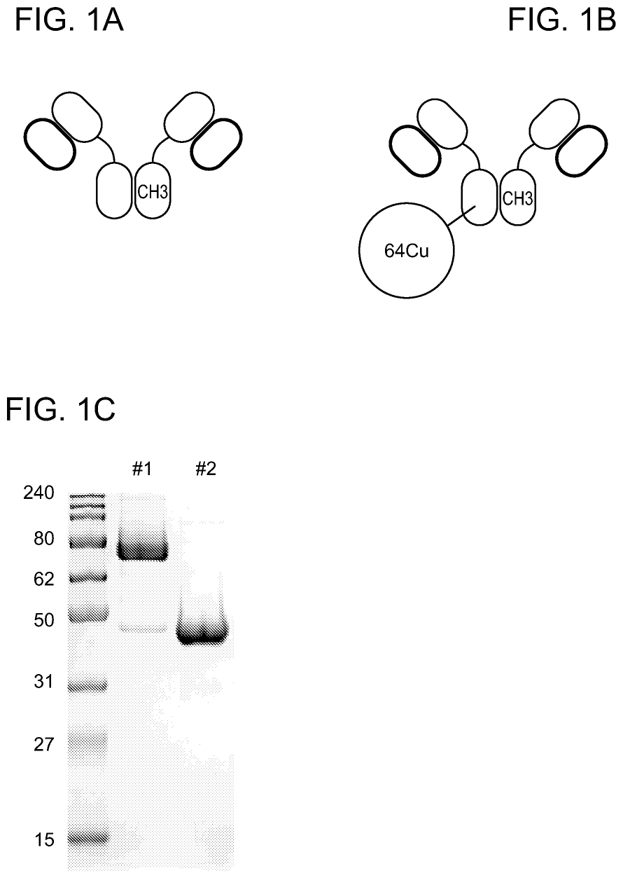

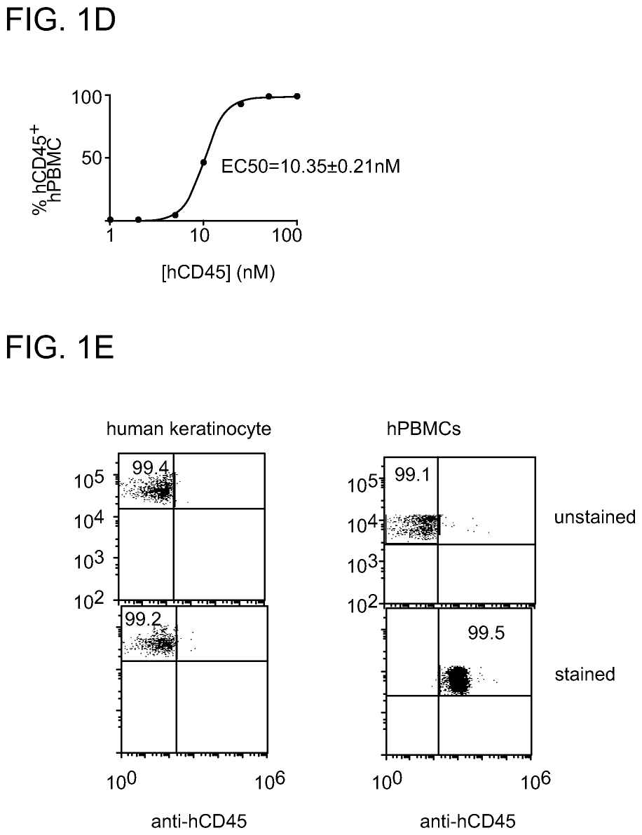

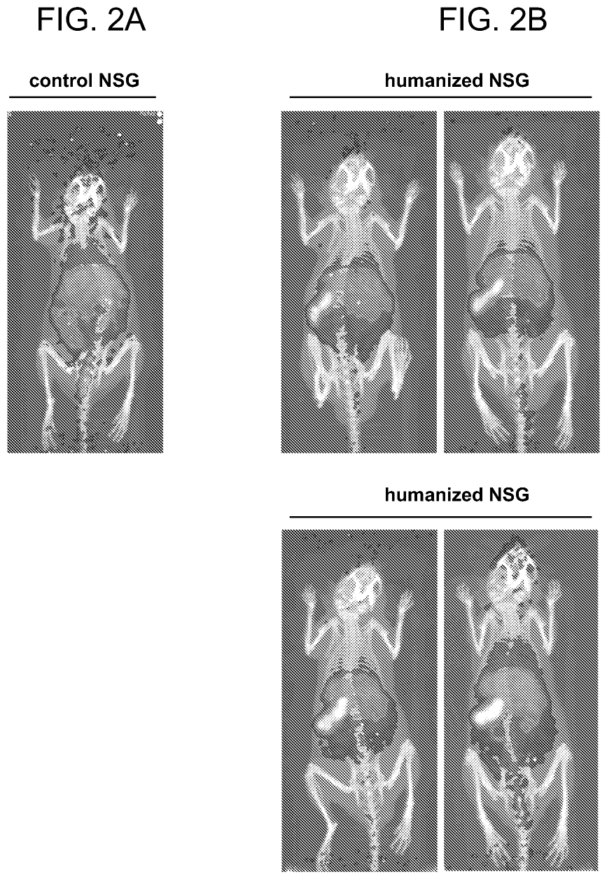

The CD45 minibody was expressed in HEK293 cells and purified. The CD45 minibody was characterized by staining human PBMCs and an EC₅₀ of ~10 nM was determined, demonstrating that anti-CD45 can be an ideal radiolabeled tracer (FIG. 1B-FIG. 1C). FIG. 1C shows the CD45 minibody protein expressed in HEK293 cells, purified via size-exclusion chromatography, and further characterized via SDS-PAGE analysis. Lane #1 of the gel shown in FIG. 1C is the CD45 minibody unheated, and lane #2 is the CD45 minibody pre-heated before loading onto the gel, confirming the dimeric nature of the CD45 minibody and the expected molecular weight. The CD45 minibody did not stain human CD45⁻ keratinocytes, confirming the specificity of the binding epitope of the CD45 minibody (FIG. 1D).

For in vivo analyses, humanized mice were prepared by injecting human PBMCs into NOD scid gamma (NSG) mice. Two weeks after injection, the CD45 minibody was radiolabeled with copper-64 (⁶⁴Cu) (referred to herein as the radiolabeled tracer), and injected into the NSG mice, which were then imaged by PET (FIGS. 2A-2B). The PET images revealed that the radiolabeled tracer could successfully detect human immune cells. The spleens of the mice were clearly detected (FIG. 2B). Control NSG mice, which did not receive human PBMCs, were injected with approximately the same amount of radiolabeled CD45 probe and did not show any signal (FIG. 2A).

These data demonstrate that the inventive radiolabeled tracer is an ideal probe for use in clinical settings, where it can be used for detection of inflammatory conditions in vivo. The CD45-specific radiolabeled tracer is well suited for imaging the behavior (dynamics, location, expansion) of macrophages and T-cells in the myocardium and in response to treatment. Macrophages and T cells are the most important immune cell subsets that are involved in the pathogenesis of many important inflammatory cardiomyopathies.

### Example 3: Anti-Murine CD45 Nanobody Probe Binds to Target with High Affinity In Vitro

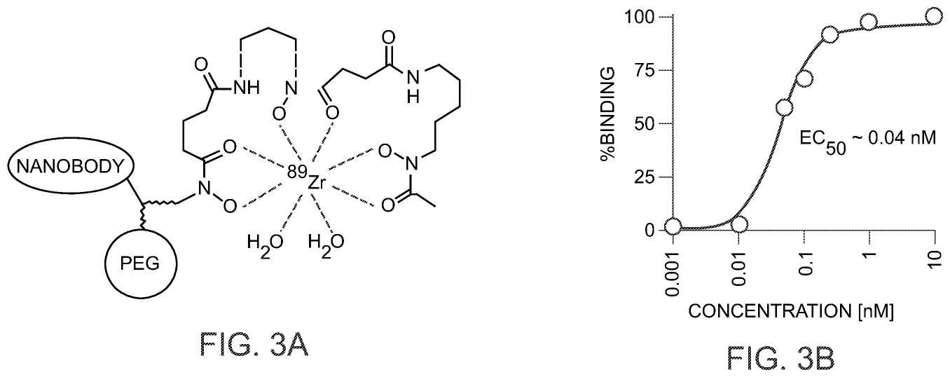

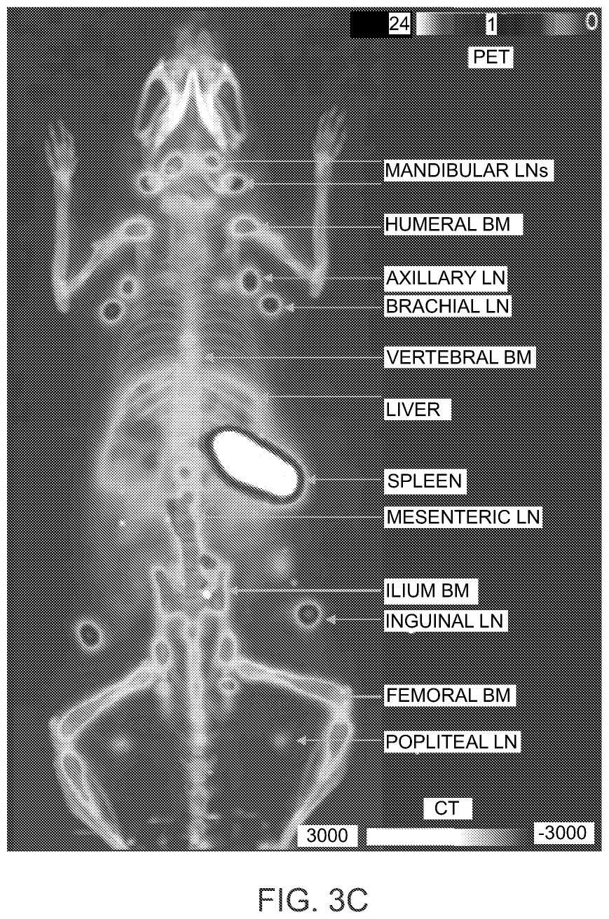

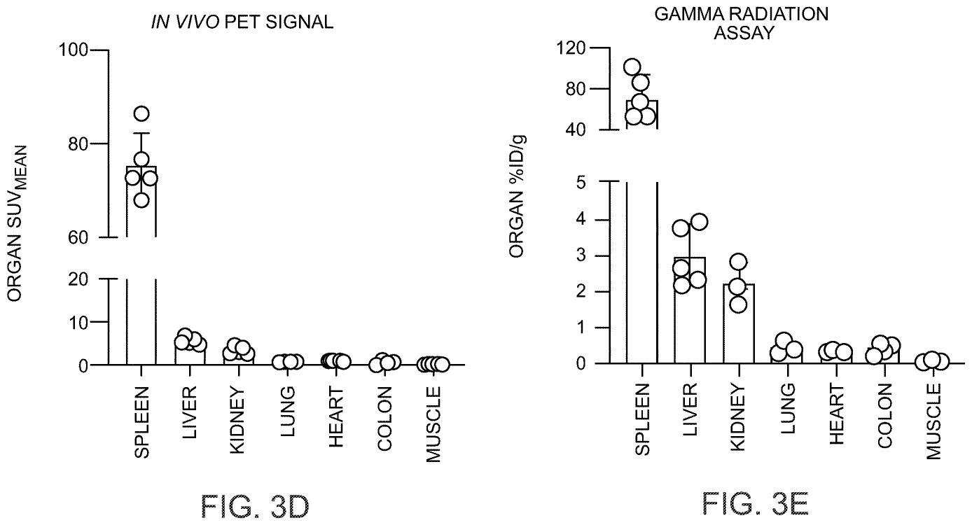

An anti-murine CD45 nanobody (also referred to as VHH) was employed as a PET probe (FIG. 3A), which was cloned, bacterially expressed, purified, and characterized using standard laboratory techniques (Teunissen et al., Star Protoc. 2(2):100434 pp. 1-19, (2021)). The nanobody was engineered with a sortase recognition motif, LPETG (SEQ ID NO: 41), at its C-terminus, enabling site-specific modification and radiolabeling (FIG. 9A). Sortase, a bacterial transpeptidase, recognizes the sortase motif, and cleaves the bond between threonine (T) and glycine (G) to form a thioester intermediate. Subsequently, the thioester is resolved by a nucleophile containing three glycines (Gly₃), yielding the final product (FIG. 9A) (Mazmanian et al., Science 285(5428):760-763 (1999). The sortase-mediated labeling ensured the functional integrity of the nanobody even after chelator and radioisotope labeling (Rashidian et al., P.N.A.S. 112(19):6146-6151; Rashidian et al., Acs Cent. Sci. 1(3):142-147 (2015)).

Staining of splenocytes with varying concentrations of the unlabeled nanobody showed that it bound murine immune cells with low nanomolar affinity, a critical characteristic for an effective PET probe (FIG. 9C). To prepare the nanobody as a PET probe, a sortase substrate containing deferoxamine (DFO) and an azide functionality was synthesized (FIGS. 3A and 9A). The DFO chelator facilitates the installation of ⁸⁹Zirconium (⁸⁹Zr), a widely used PET isotope in the clinic, which has a half-life of approximately 3.3 days. The azide functionality allows the installation of a polyethylene glycol (PEG) molecule into the nanobody that can be used to decrease non-specific kidney uptake and enhance image signal-to-noise ratios (Rashidian et al., J. Exp. Med. 214(8):2243-2255 (2017)). Accordingly, the anti-murine CD45 nanobody was site-specifically labeled with the Gly₃-DFO-azide sortase substrate and subsequently installed with a PEG moiety via the azide-alkyne click chemistry (FIGS. 3A and 9A). The reaction resulted in the generation of DFO-labeled and PEGylated nanobody with a near quantitative yield (FIGS. 3A and 9B). To evaluate whether addition of a Zr ion or PEG labeling impacted the nanobody's binding to immune cells, the DFO-PEG labeled nanobody underwent Zr ion installation through the addition of a Zirconium (IV) chloride (ZrCl₄) solution. Subsequently, different concentrations of the Zr-DFO-PEG-labeled nanobody were assessed for murine splenocytes binding. Flow cytometric analyses demonstrated that the Zr-DFO-PEG-labeled nanobody stained murine splenocytes with low nanomolar affinity, which, without being bound by theory, suggests that the sortase-mediated site-specific labeling as well as installation of the Zr ion did not compromise the nanobody's binding capability (FIG. 3B).

### Example 4: ⁸⁹Zr-CD45 Nanobody PET Probe Visualizes Immune Cell-Rich Organs with High Clarity In Vivo

Next, the ability of the probe to visualize immune cell-rich organs in vivo was evaluated. To achieve this, DFO-labeled nanobodies were created with varying PEG sized molecules, including no PEG, 10 kDa PEG (PEG10), and 20 kDa PEG (PEG20). These three variations of the CD45-PET probe were then installed with a with an ⁸⁹Zr radioisotope following established protocols (Teunissen et al., Star Protoc. 2(2):100434, pp. 1-19 (2021)) and injected into healthy immunocompetent C57BL/6 mice (~50 μCi dose, n=2 for each group). Mice then underwent PET/CT imaging at several time points post-injection of the probe (2 hours for no PEG, 24 hours for PEG10 and PEG20, due to their higher circulatory half-lives). CD45-PET imaging revealed whole body visualization of the immune system, with clear uptake in the spleen, bone marrow and all major lymph node chains (FIG. 3C and FIG. 9D). However, immune cell-rich organs displayed superior uptake with the installation of PEG20. Moreover, the use of PEG20 led to the least amount of non-specific uptake in kidneys (FIGS. 9D-9E), aligning with findings from the previously reported studies (Rashidian et al., J. Exp. Med. 214(8):2243-2255 (2017); Rashidian et al., Proc. Natl. Acad. Sci. U.S.A 116(34):16971-16980 (2019)). Therefore, the ⁸⁹Zr-CD45 nanobody was installed with PEG20 (termed “CD45-PET probe”) for the following studies. Compared to immune cell-rich organs, such as the spleen, the in vivo physiologic mean standard uptake value (SUVₘₑₐₙ) of lower immune cell density organs (e.g., liver, kidney, lung, heart, colon, and muscle) was significantly lower (FIGS. 3C-3D). After in vivo imaging, animals were euthanized, organs of interest were dissected and underwent confirmatory gamma radiation assay. The percentage of injected dose per gram of tissue (% ID/g) validated the in vivo findings (FIG. 3E). To confirm that the signal was specific to the CD45 nanobody, mice were given a pre-injection of non-radiolabeled (“cold”) CD45 nanobody (300 μg, i.p.) two hours prior to injection with the CD45-PET probe. When compared with mice with no pre-injected cold protein, a significant decrease in immune organ signal was observed (FIGS. 9F and 9G), confirming the specificity of the CD45-PET probe. Given that physiologic signal was low across these major organ systems, the CD45-PET approach was assessed if it could detect pathogenic immune infiltration in preclinical models of inflammation.

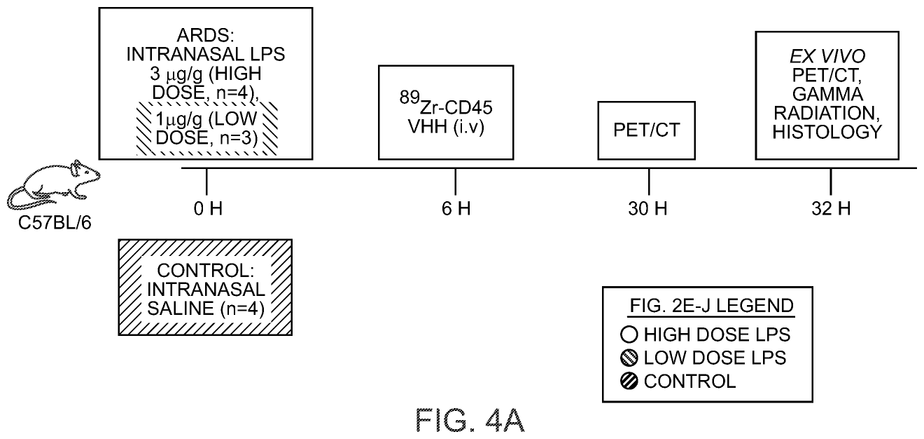

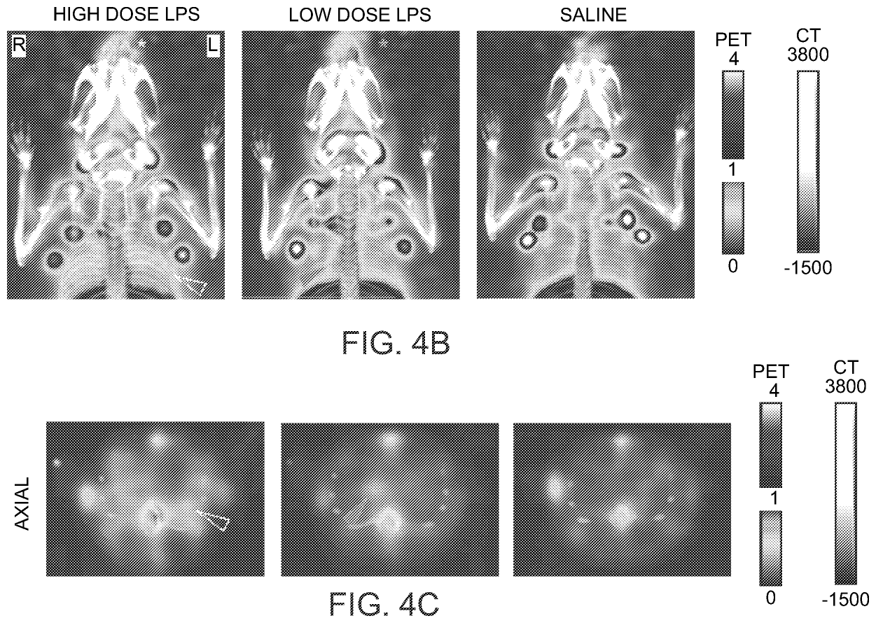

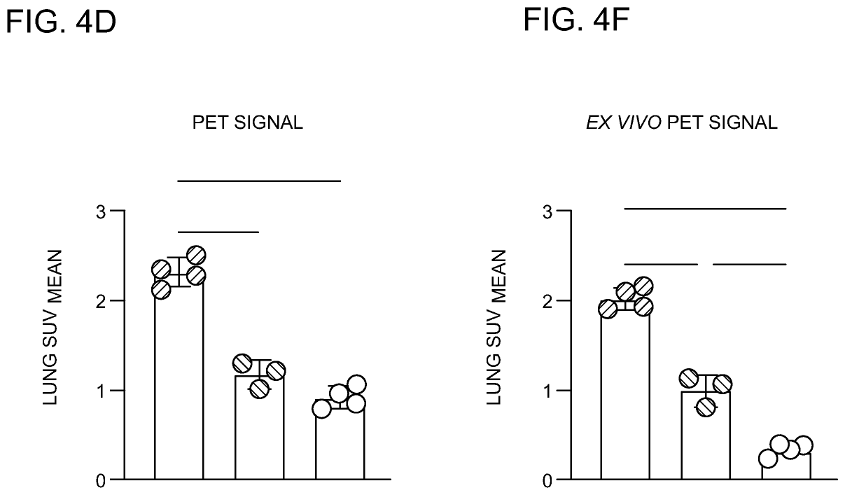

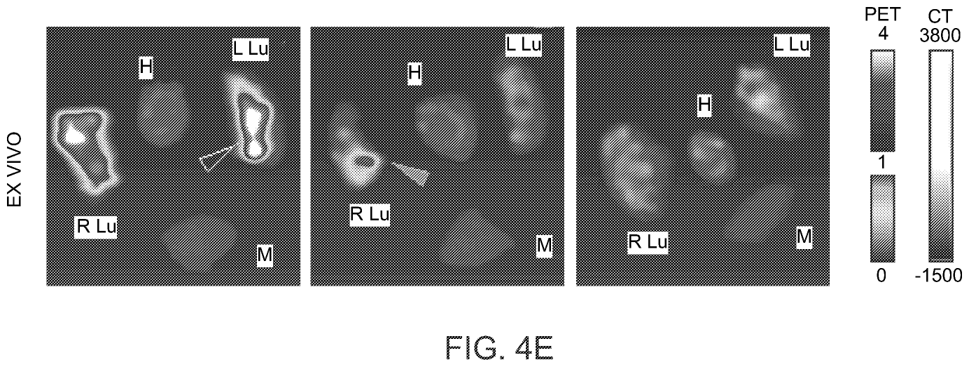

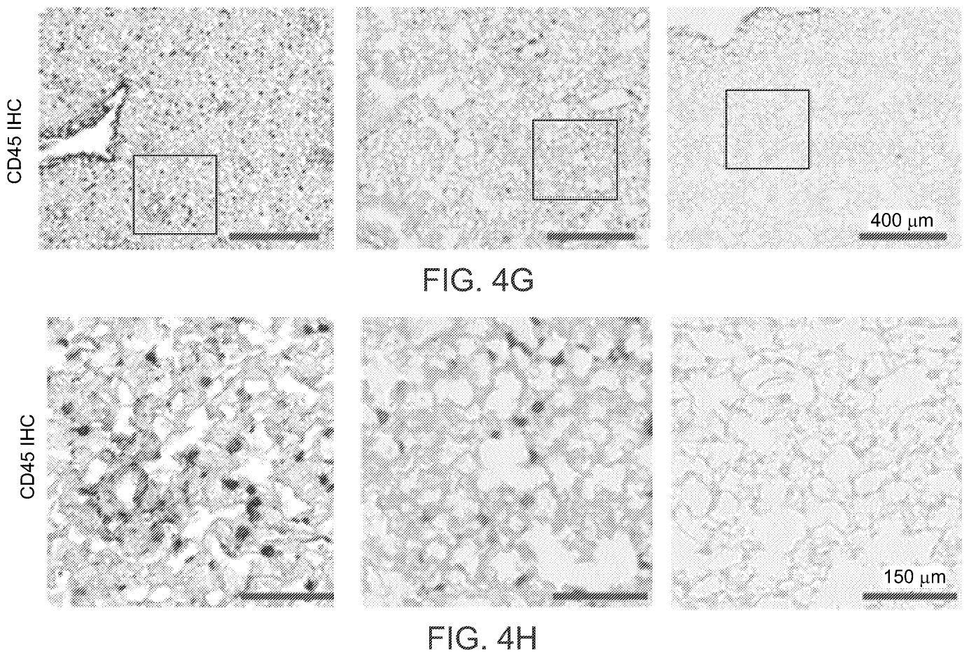

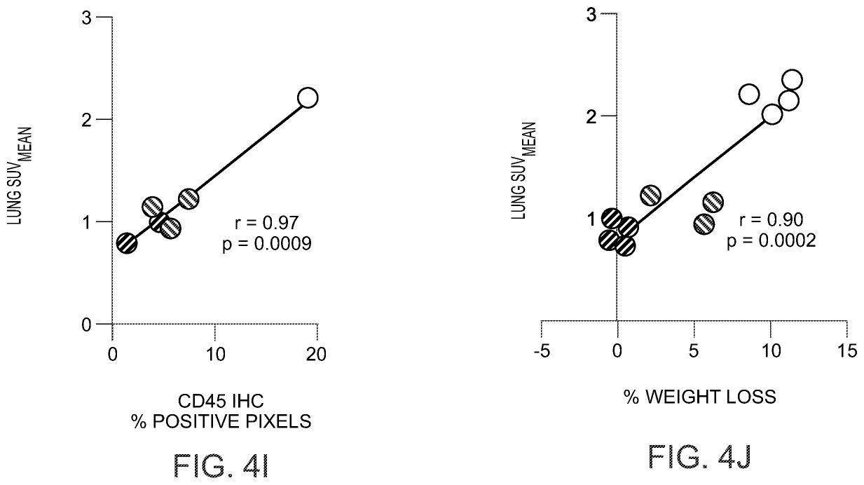

The CD45-PET signal directly correlated with both degree of inflammatory infiltrate and clinical severity in pulmonary inflammation model. Acute and chronic inflammatory lung conditions, such as acute respiratory distress syndrome (ARDS), chronic obstructive pulmonary disease (COPD), and interstitial lung diseases, are clinically evaluated with imaging techniques such as CT (Pauwels et al., Am J Respir Crit Care Med. 163(5):1256-1276 (2001)). However, much of their underlying pathogenesis remains poorly understood, in part because histopathologic sampling with lung biopsy is rarely performed due to its invasiveness and lack of reproducibility (Cottin, Eur. Respir. J. 48(6):1274-1277 (2016)). Thus, CD45-PET was first tested in a murine model of ARDS, which produces a robust neutrophil and macrophage-driven inflammatory response in the lungs. Immunocompetent C57BL/6 mice were intranasally administered high (3 μg/g, n=4) or low (1 μg/g, n=3) doses of lipopolysaccharide (LPS) or saline (control, n=4) (Khadangi et al., Sci. Rep. 11(1):7777, pp. 1-12 (2021)). Six hours following LPS administration, mice were intravenously injected with the CD45-PET probe, followed by in vivo PET/CT imaging conducted 24 hours post-probe injection with subsequent lung dissection, ex vivo PET/CT imaging, gamma radiation assay and histologic validation (FIG. 4A). Mice were weighed before LPS administration and after in vivo imaging.

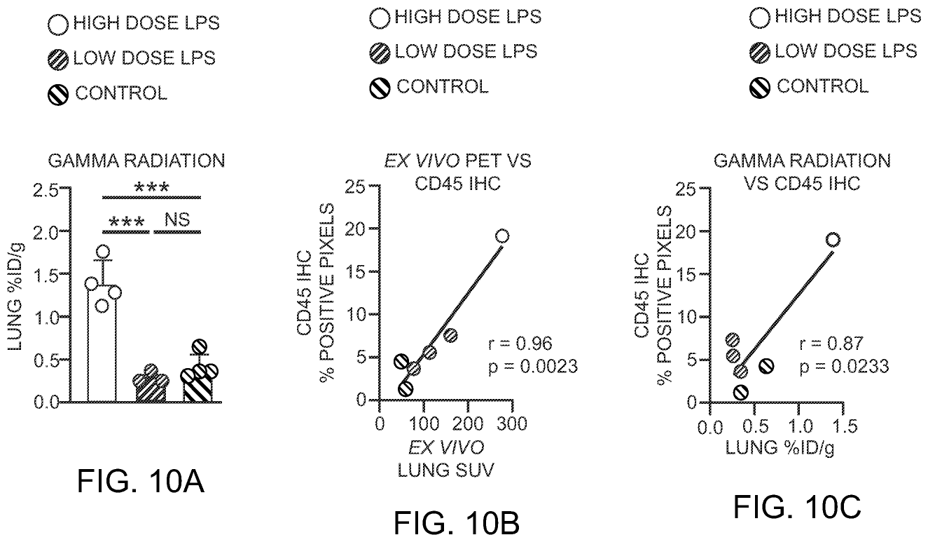

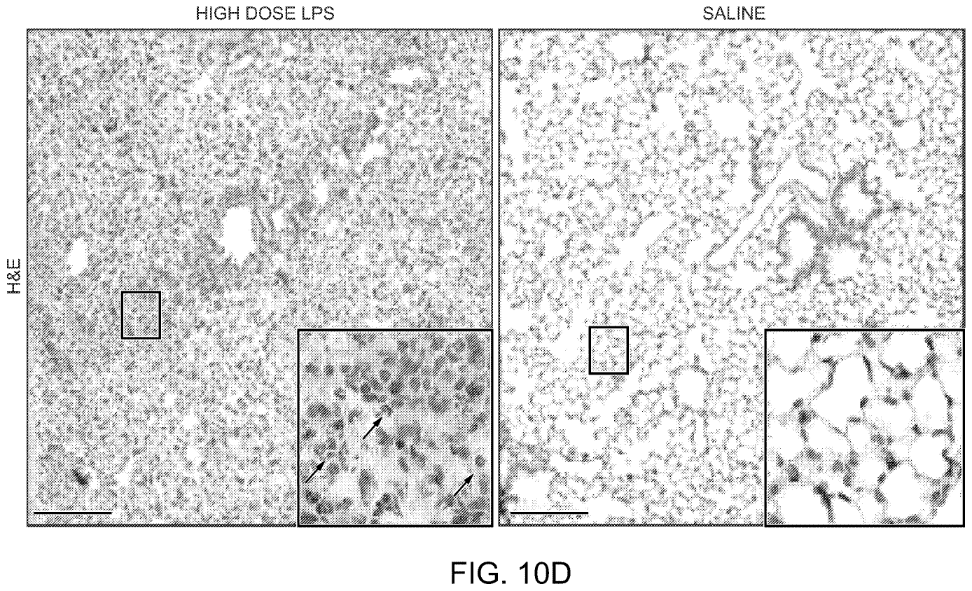

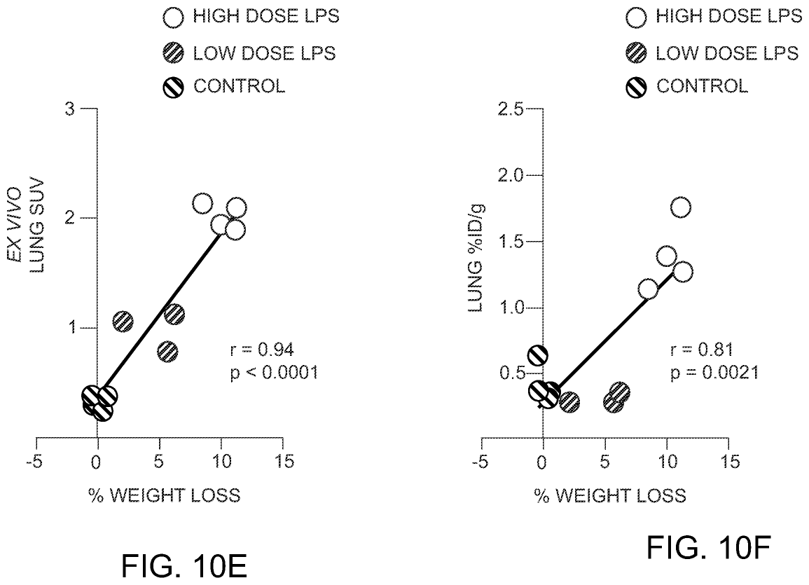

CD45-PET imaging revealed significantly increased probe uptake in the lungs of LPS-exposed mice in a dose-dependent fashion (FIGS. 4B-4C). Mice given a higher dose of LPS showed more diffuse signal extending throughout both lungs, whereas mice given a lower dose of LPS showed more focal areas of signal (FIGS. 4B-4D). Quantitatively, in vivo PET analysis showed that high dose LPS mice had lung SUVₘₑₐₙ (2.32±0.15) that was more than 2-fold higher than control mice (0.92±0.12, p<0.0001) and approximately 1.5-fold higher than low dose LPS mice (1.17±0.15, p<0.0001) (FIG. 4C). Differences in signal distribution in the lung in vivo were confirmed with ex vivo imaging of dissected lungs (FIG. 4D). Quantitative analysis of ex vivo PET/CT lung images validated the in vivo findings, with greater segregation of signal intensity between the low dose and control mice than seen with in vivo quantification analyses (FIG. 4D). Dissected lungs then an underwent gamma radiation assay, which further confirmed the in vivo and ex vivo analyses (FIG. 10A).

To assess whether PET probe uptake reflected true immune cell infiltration, a portion of lung samples underwent histological analysis with CD45 immunohistochemistry and hematoxylin and eosin (H&E) staining (FIG. 4E). Sections were imaged and quantified using previously described methods (Freise et al., J. Nucl. Med. 59(6):980-985 (2018); Koshkaki et al., J. Pers. Med. 10(3):112, pp. 1-16 (2020)). In brief, after subtracting background, the percentages of pixels staining positively for CD45 were calculated by two independent blinded investigators across three individual 10× brightfield images per lung and averaged. All samples were evaluated. It was observed that there was a high degree of correlation between CD45⁺ staining and in vivo lung probe uptake (Pearson r=0.97, p=0.0009), ex vivo lung probe uptake (Pearson r=0.96, p=0.0023) and gamma radiation (Pearson r=0.87, 0.0233) (FIGS. 4F and 10B-10C). Consistent with previous studies of this model, H&E staining revealed markedly disrupted tissue architecture alongside a neutrophilic inflammatory infiltrate exposed to LPS (FIG. 10D).

Finally, whether PET signal intensity varied with severity of disease was examined. Using weight loss as a proxy for disease status, an impressive correlation between the intensity of CD45-PET signal and the severity of the disease was observed (Pearson r=0.90, p=0.002; based on in vivo PET-quantification analyses, FIG. 4G). This observation was reproducible across both ex vivo and gamma radiation assay data (FIGS. 10E-10F). Collectively, without being bound by theory, these results highlight the robustness and reliability of CD45-PET imaging in accurately detecting and quantifying lung inflammation, with the imaging signal intensity strongly correlating with the severity of the disease (Bosmann et al., Faseb J. 26(5):2137-2144 (2012)).

### Example 5: CD45-PET Detects Inflammation in a Murine Model of an IBD, with Signal Strongly Correlating with Disease Severity and Inflammatory Infiltrate

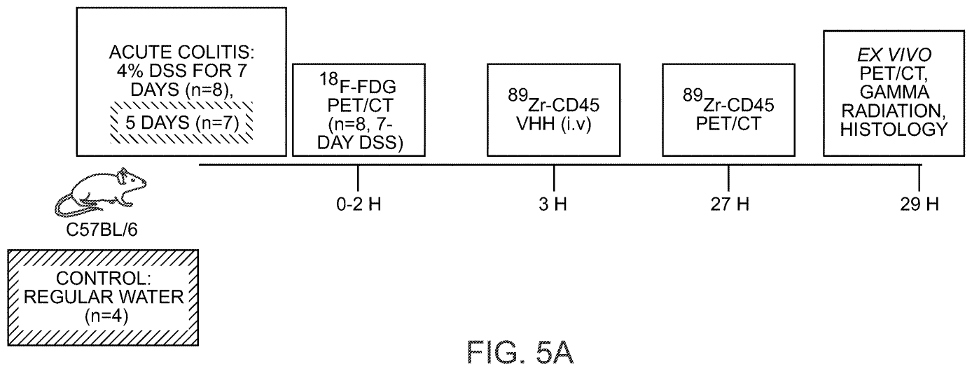

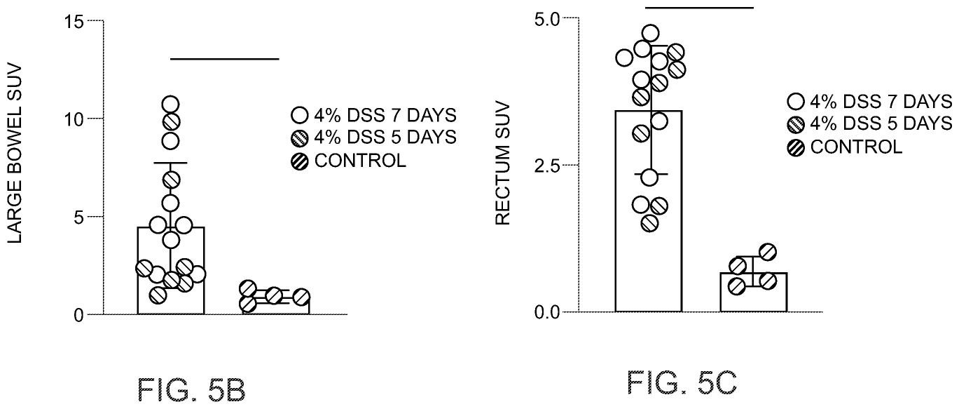

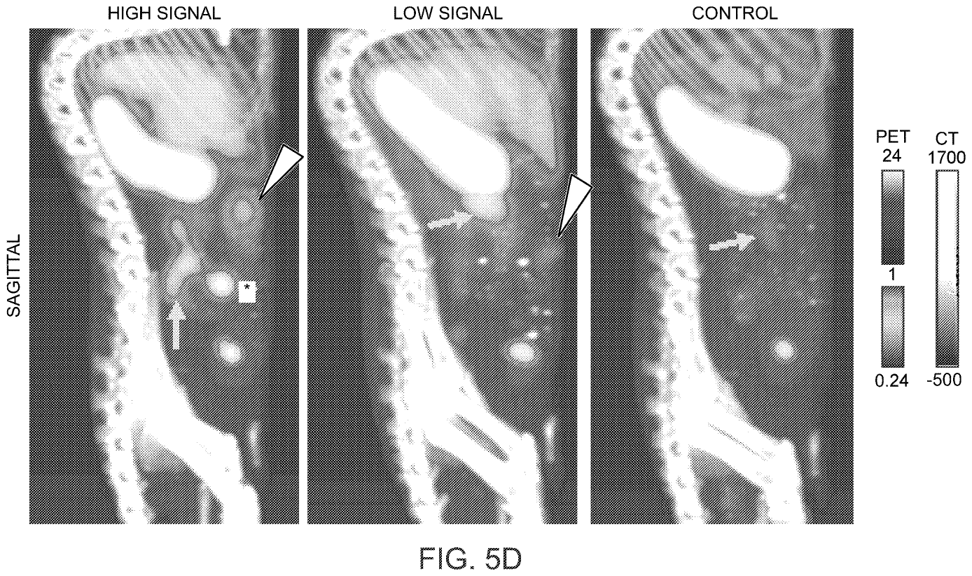

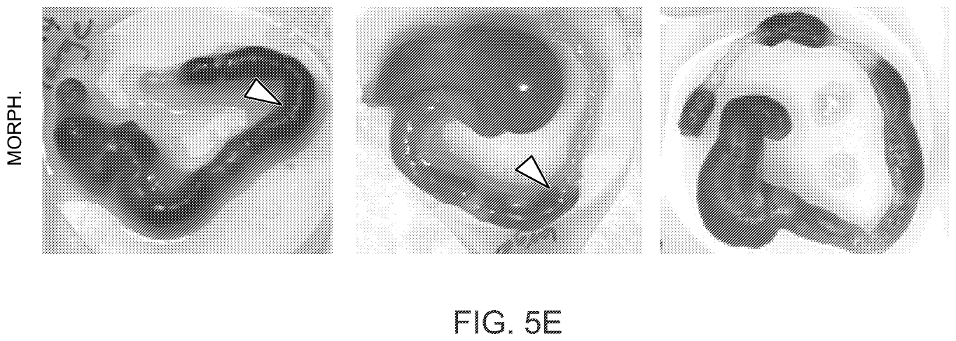

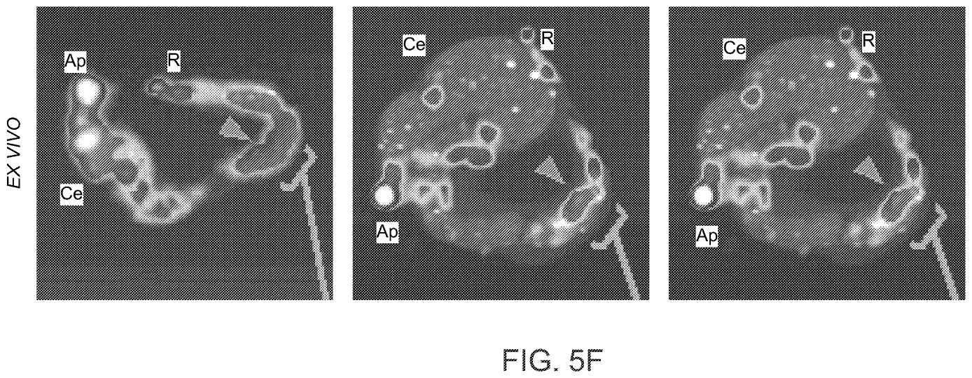

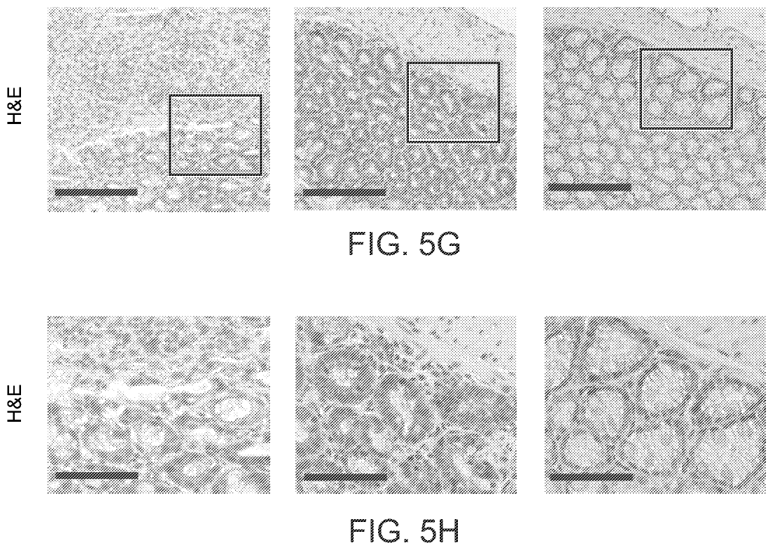

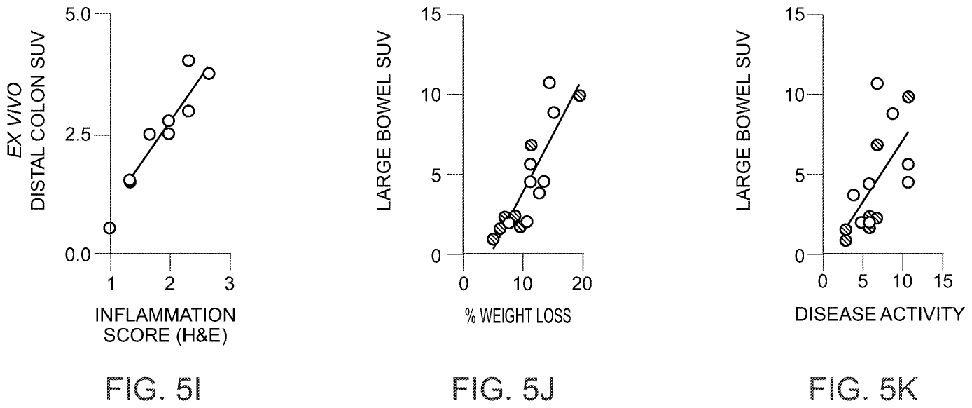

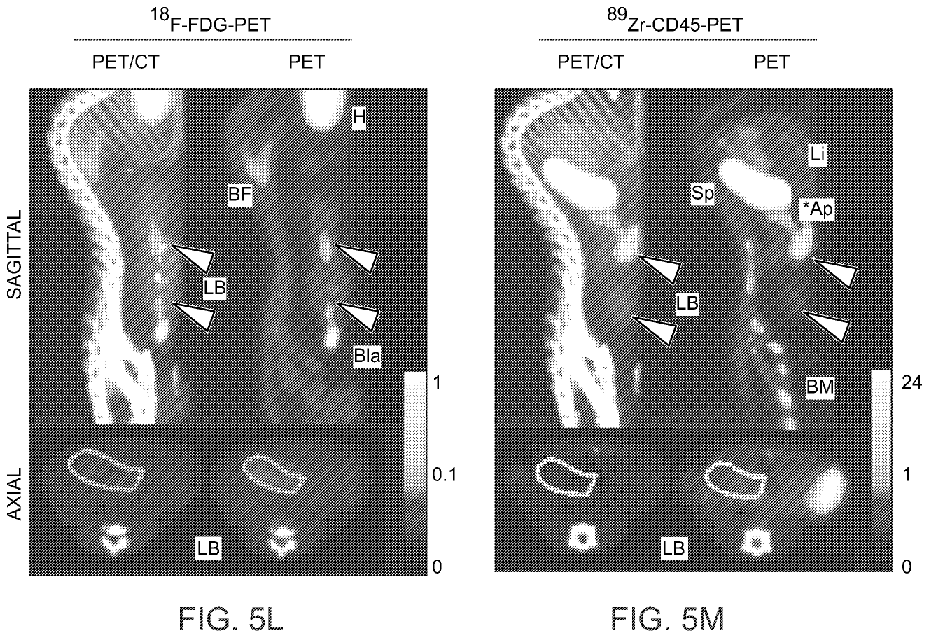

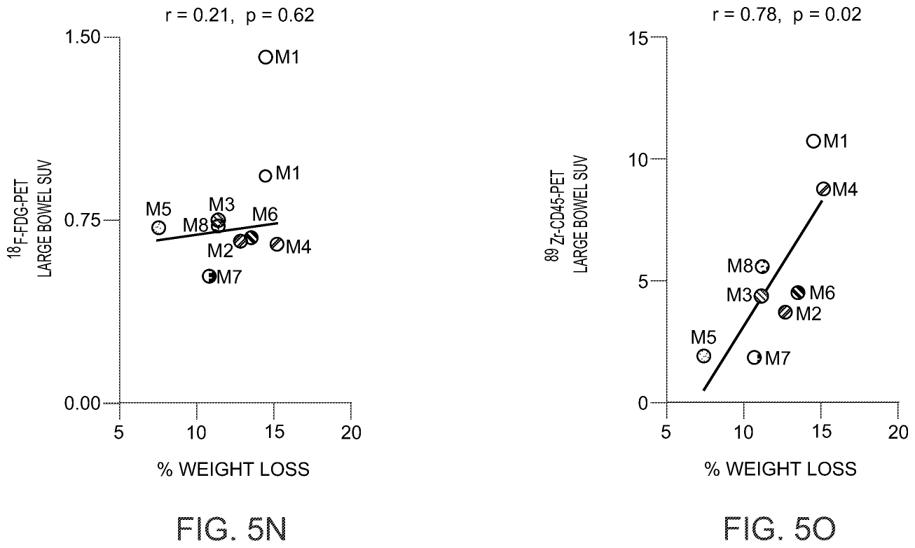

Dextran sodium sulfate (DSS) induces colitis in a well-established preclinical model by disrupting the intestinal barrier and triggering an immune response driven primarily by neutrophils and macrophages (Chassaing et al., Curr. Protoc. Immunol. 104:15, pp. 1-16 (2014)). Acute colitis was induced by giving immunocompetent C57BL/6 mice drinking water supplemented with 4% DSS for 7 days (n=8) or 5 days (n=7) (FIG. 5A). Colitis disease activity index (DAI), which graded the degree of weight loss, stool consistency, and bleeding (Kim et al., J. Vis. Exp. (60):3678, pp. 1-6 (2012)), was evaluated every other day. At the end of the DSS exposure period, mice underwent CD45-PET imaging. Mice in the 7-day exposure group also underwent ¹⁸F-FDG-PET imaging prior to CD45-PET probe injection to allow for head-to-head comparison (FIG. 5A).

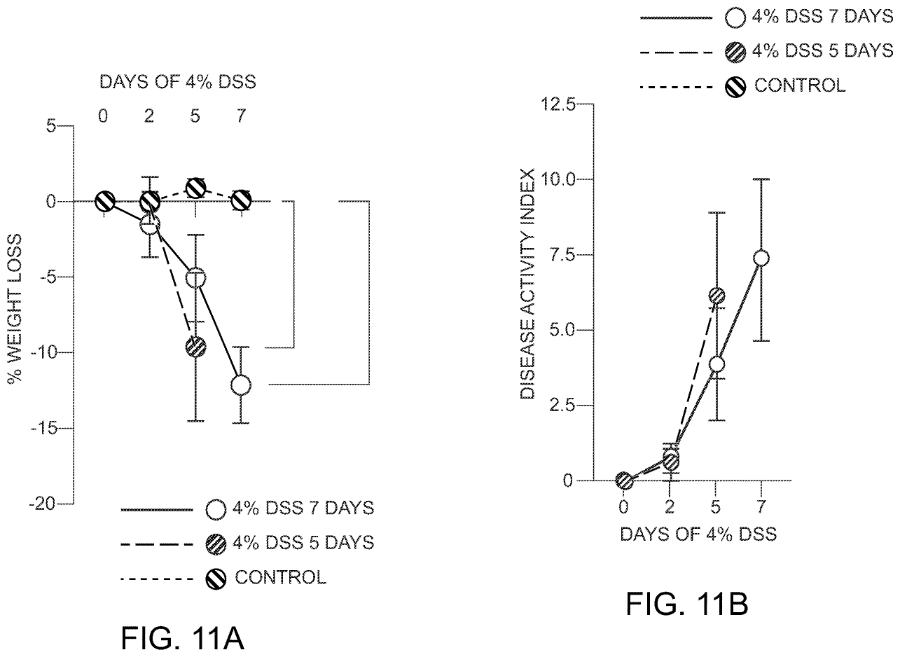

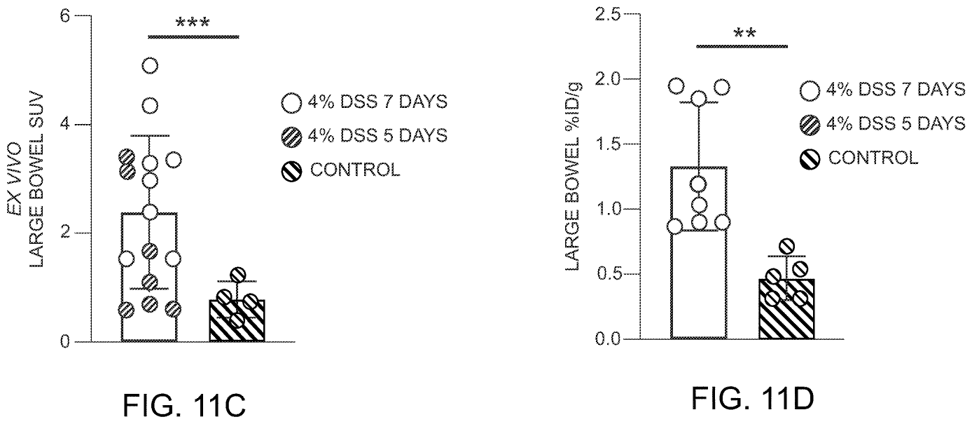

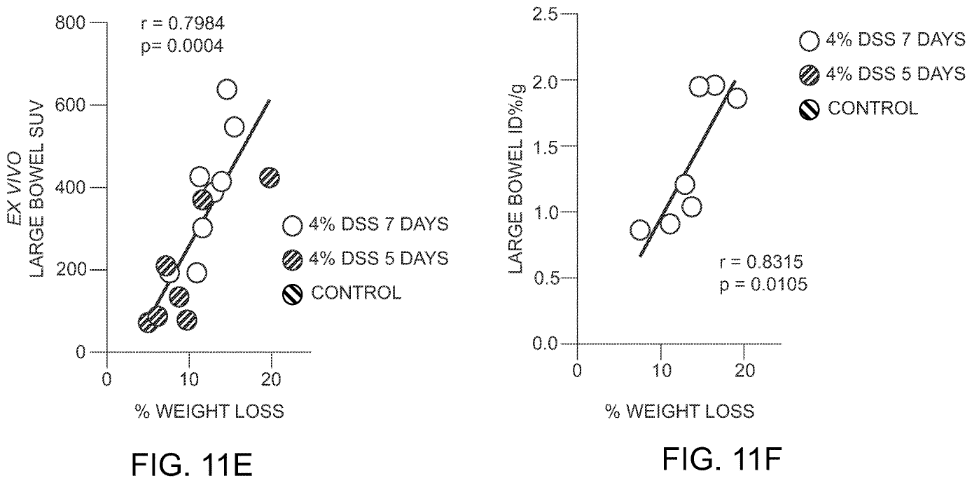

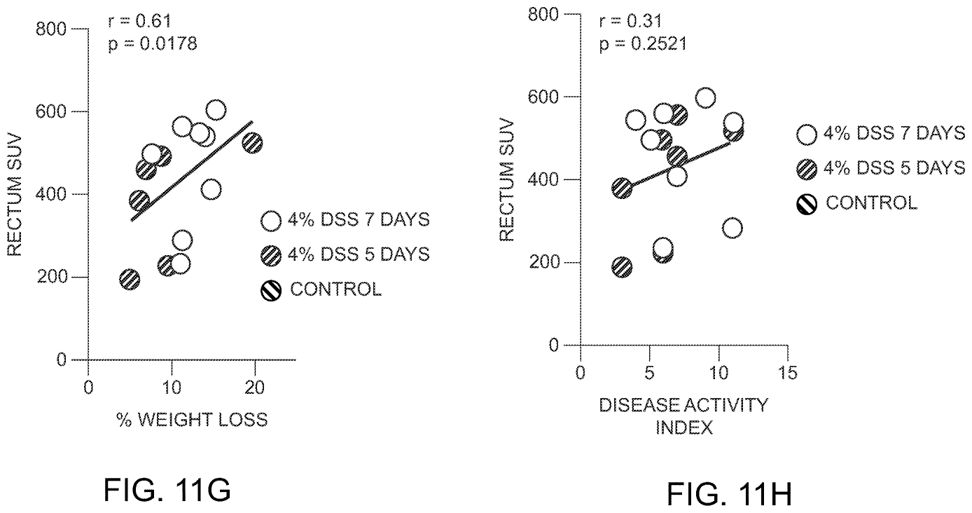

Mice that received 4% DSS lost significantly more weight than controls at both the 5- and 7-day endpoint (FIG. 11A). Disease severity was heterogenous at experiment endpoints, with mean DAI scores of 6.1±2.7 (range: 3-11, n=7) on Day 5 and 7.7±2.7 (range: 4-11, n=8) on Day 7 (FIG. 11B). In vivo PET image analysis revealed that SUVₘₑₐₙ in the large bowel of mice given 5 days (3.6±3.4) and 7 days (5.2±3.1) of 4% DSS was significantly higher than controls (0.79±0.33, p=0.0006) (FIG. 5B). This was confirmed with ex vivo and gamma radiation assay analysis (FIGS. 11C-11D). As shown in FIG. 5B, each DSS group had significant variability in large bowel SUVₘₑₐₙ. In vivo rectum SUVₘₑₐₙ was also significantly higher in the 5-day cohort (3.7±1.1) and 7-day cohort (3.2±1.2) compared to controls (0.69±0.26, p<0.0001) (FIG. 5C), but with less heterogeneity in signal compared to large bowel.

When mice were segregated by large bowel PET signal, where “high signal” mice were defined as greater than one standard deviation above the mean, distinct differences in several disease severity measures was observed as compared to “low signal” mice. In addition to having higher bowel signal, higher signal mice featured increased mesenteric lymph node uptake as well as appendiceal uptake compared to low signal mice (FIG. 5D). Morphologic assessment revealed that the high signal mice had notable anatomical sequelae of inflammation, including bowel wall edema, hemorrhage, and bowel shortening compared to the low signal and control mice (FIG. 5E). Ex vivo imaging confirmed the difference in signal intensity observed in vivo. Further, ex vivo images were used to select bowel segments for histologic evaluation (FIG. 5F). H&E was performed to evaluate immune infiltration in specific sections of the bowel shown in FIG. 5F (FIGS. 5G-5H). Inflammation scoring showed that the strength of ex vivo PET signal indeed correlated with the degree of immune infiltration in that same bowel segment (FIG. 5I).

Given the observed heterogeneity in PET signal and DAI scores, the PET signal intensity in the large bowel or rectum and the association with disease severity was investigated. Higher in vivo PET signal in the large bowel was tightly correlated with weight loss (Spearman r=0.89, p<0.0001) and disease activity score (Spearman r=0.79, p=0.0008) (FIGS. 5J and 3K). Ex vivo and gamma radiation were also significantly correlated with weight loss (FIGS. 11E and 11F). Higher in vivo PET signal in the rectum was also correlated with weight loss (Spearman r=0.61, p<0.0178) but not disease activity score (Spearman r=0.31, p=0.2521) (FIGS. 11G and 11H).

### Example 6: CD45-PET Outperforms 18F-FDG-PET in Stratifying Colitis Disease Severity

¹⁸F-FDG-PET has emerged as a valuable tool for diagnosing and monitoring IBD, particularly in pediatric patients for whom invasive endoscopy procedures may be less suitable (Lemberg et al., Inflamm. Bowel Dis. 11(8):733-738 (2005)) However, non-specific uptake due to peristalsis, medications, or the gut microbiome may obscure bowel inflammation (Cronin et al., Br. J. Radiol. 85(1017):1211-1221 (2012)). Thus, a head-to-head comparative analysis of the CD45-PET probe against ¹⁸F-FDG was conducted in the 7-day 4% DSS cohort of mice (n=8). As shown in FIG. 5A, 8 mice were exposed to DSS for 7 days to induce acute colitis, while 4 control mice were given regular drinking water. On Day 7, DSS-treated mice and control mice were i.v. injected with ~85 μCi ¹⁸F-FDG and imaged after 90 minutes. After ¹⁸F-FDG-PET imaging was complete, mice were injected with the CD45-PET probe and imaged 24 hours later. By this time, the ¹⁸F had undergone several (>12) decay half-lives, and all PET signal could be attributed to the CD45-PET probe.

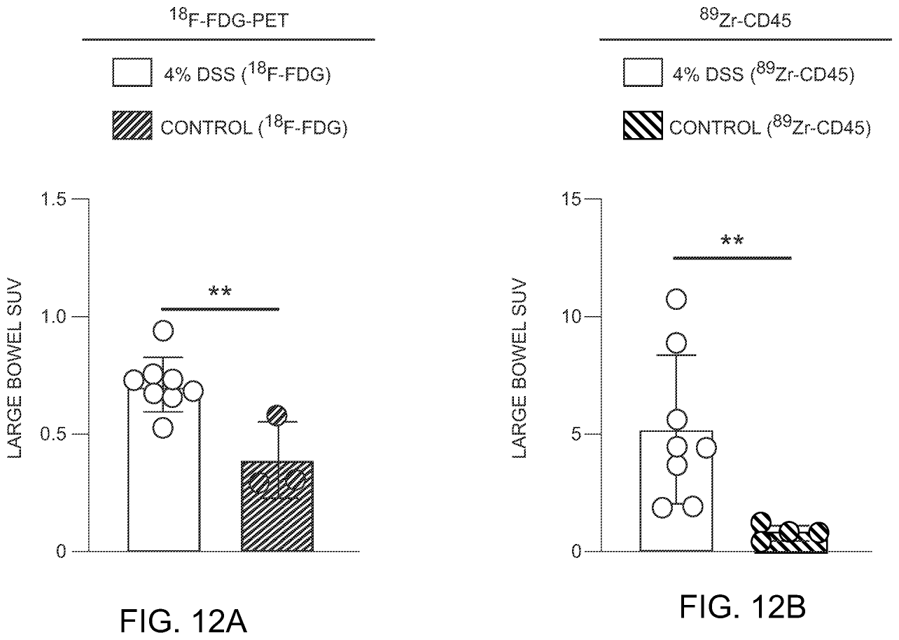

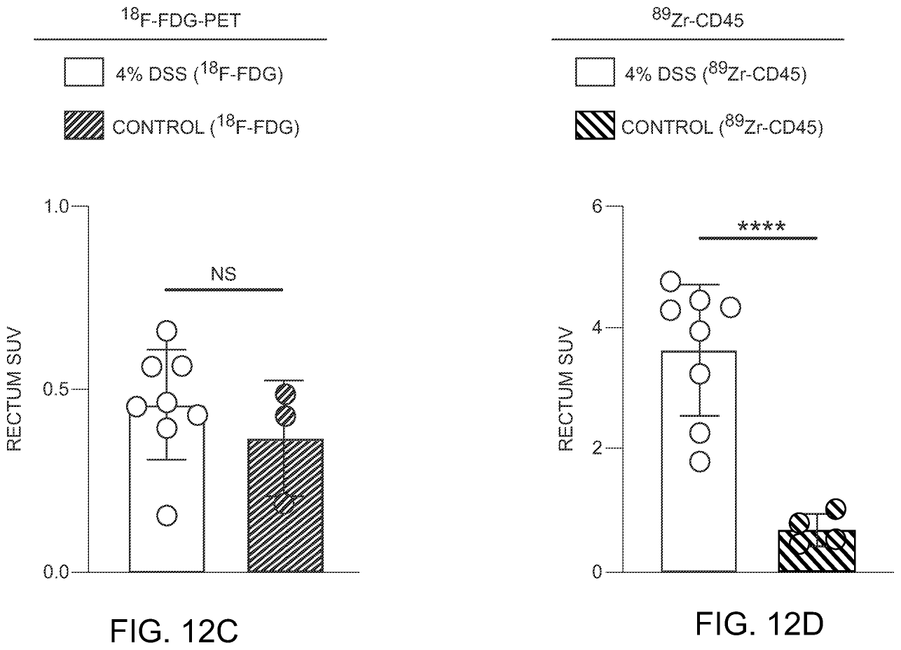

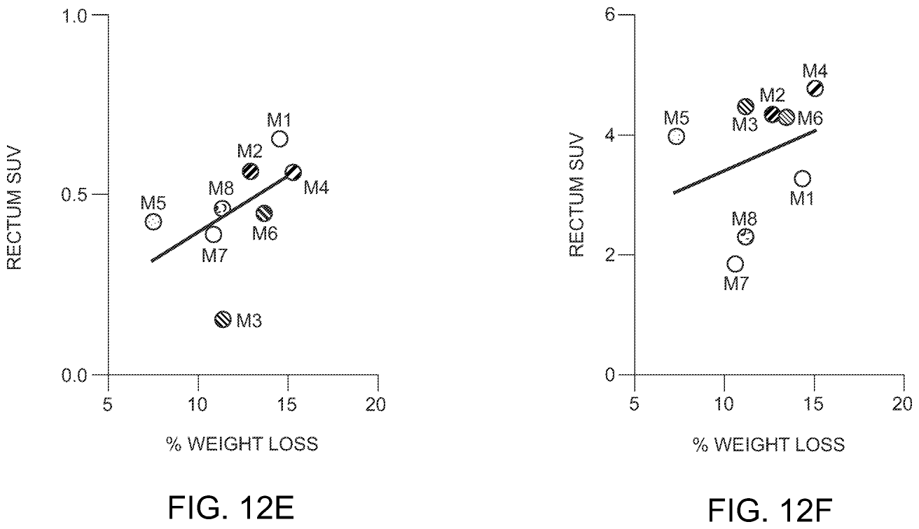

FIGS. 5L and 5M show representative images from ¹⁸F-FDG-PET versus CD45-PET in the same mouse after 7 days of DSS exposure. With ¹⁸F-FDG-PET treatment, diffusely increased signal was observed throughout the large bowel, while CD45-PET treatment showed more focal uptake in the same area of the bowel (axial images of FIGS. 3L and 5M). Consistent with previously published studies (Bettenworth et al., J. Nucl. Med. 54(5):748-755 (2013)), ¹⁸F-FDG-PET uptake in the large bowel was significantly higher in the DSS mice (SUVₘₑₐₙ 0.71±0.11) compared to controls (0.39±0.16) (p=0.0047) (FIG. 12A). CD45-PET similarly detected significant differences in large bowel uptake in vivo between DSS-exposed mice and controls (5.2±3.1 and 0.79±0.33, respectively; p=0.0006) (FIG. 12B). While CD45-PET detected significant differences in the rectum between groups, ¹⁸F-FDG-PET did not (FIGS. 12C and 12D). A crucial distinction between these two PET probes was shown by correlation analysis of weight loss (a proxy for disease severity) and signal intensity in the large bowel. In contrast to the CD45-PET probe (Pearson r=0.78, p=0.02), ¹⁸F-FDG-PET signal did not correlate with disease severity (Pearson r=0.21, p=0.62) (FIGS. 5N and 5O). In the rectum, neither modality showed correlation between PET probe uptake in the rectum and weight loss (FIGS. 12E and 12F).

Taken together, these findings demonstrate that CD45-PET reliably detects inflammation in a murine model of colitis, with areas of PET probe uptake correlating to anatomic and histologic evidence of immune infiltration. Moreover, CD45-PET signal reflects disease severity across multiple parameters, including, weight loss and a validated disease activity index-outperforming ¹⁸F-FDG-PET.

### Example 7: CD45-PET Imaging Visualizes Immune Cells Dynamics Longitudinally in Colitis Model

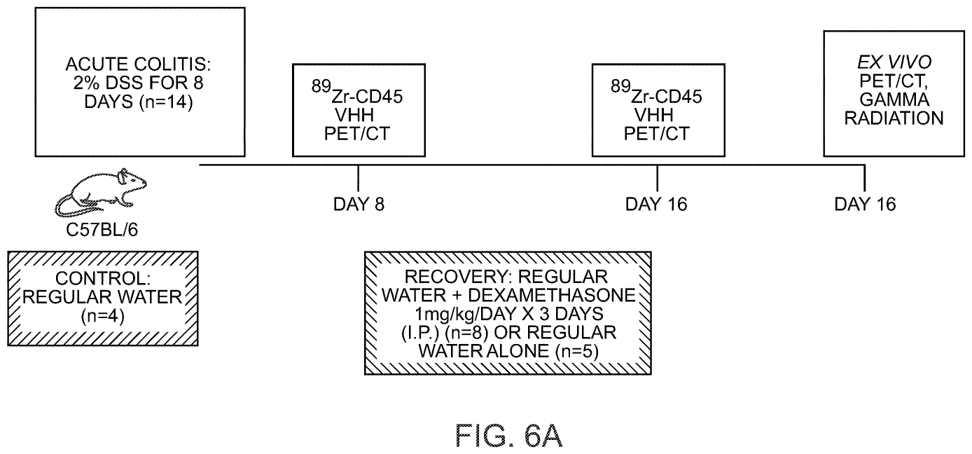

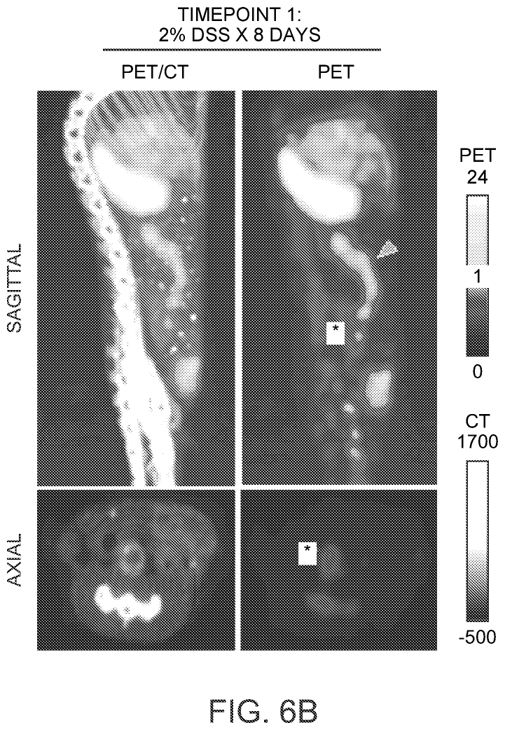

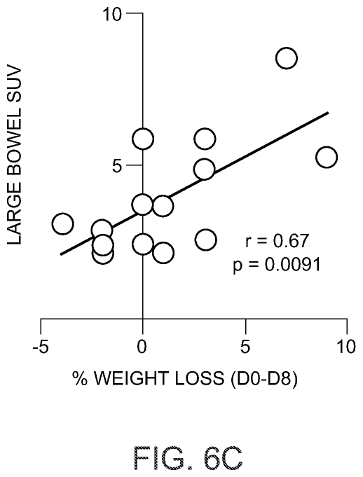

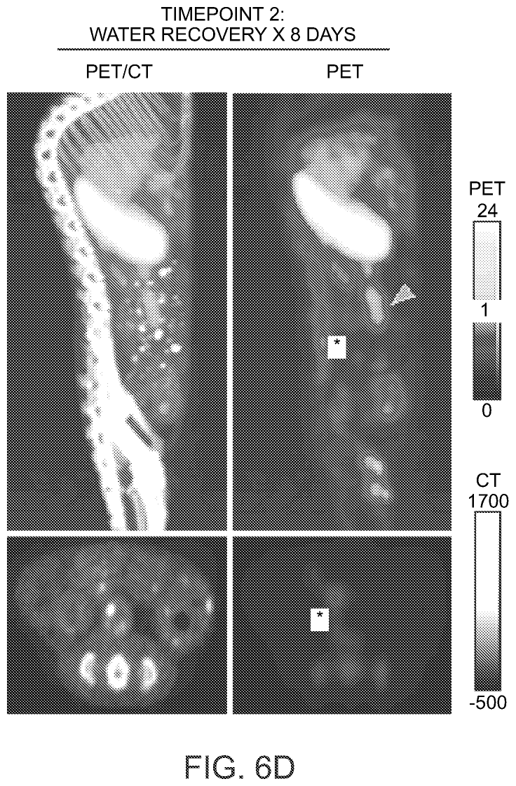

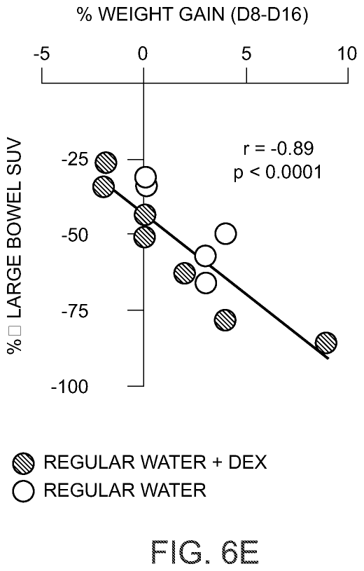

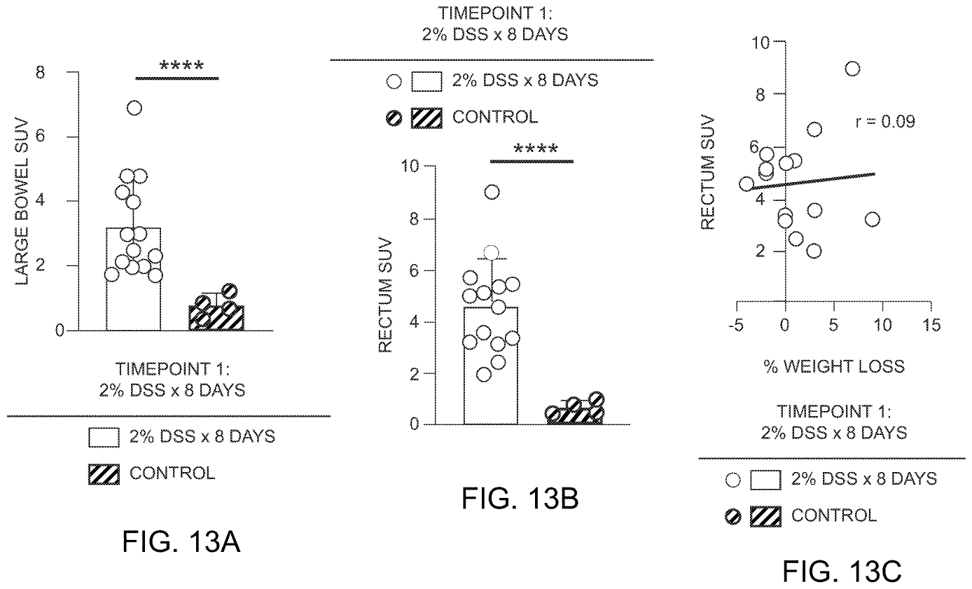

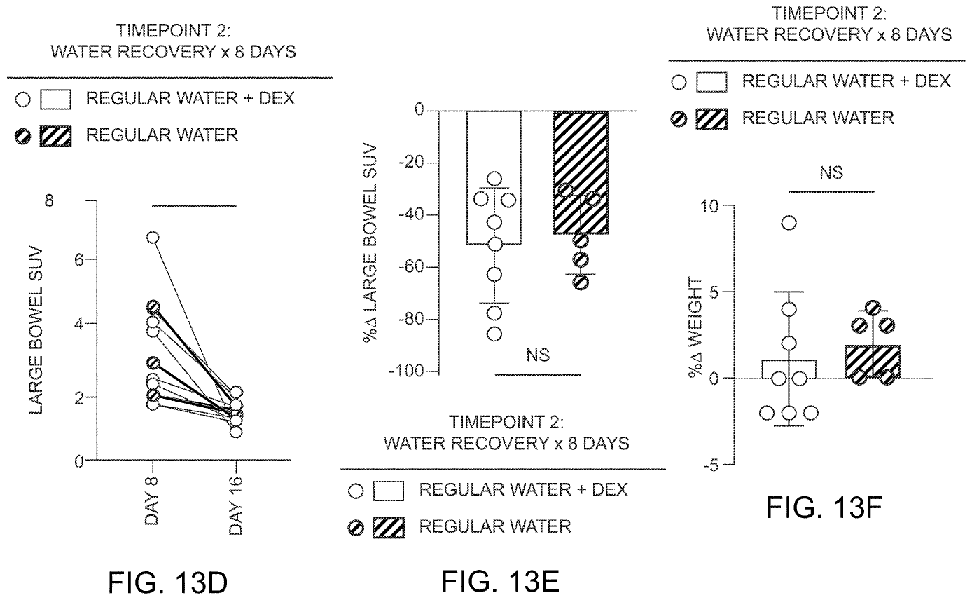

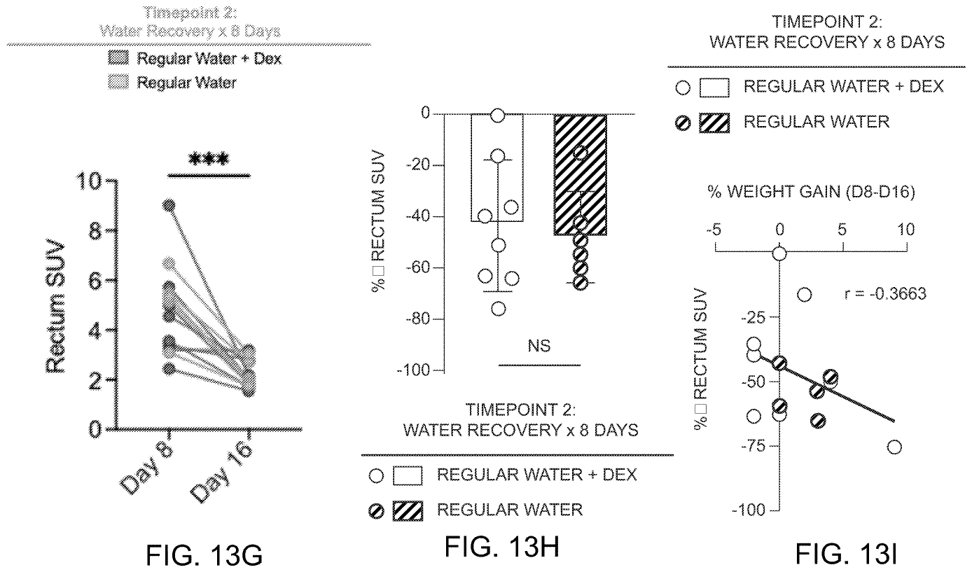

After establishing that CD45-PET clearly detected inflammation in DSS-induced colitis, it was then explored whether CD45-PET can monitor immune cell dynamics in vivo with serial imaging. Accordingly, mice were exposed to 2% DSS for eight days (n=14) before undergoing their first timepoint of CD45-PET imaging (FIG. 6A). As with the 4% DSS cohorts, CD45-PET clearly visualized increased large bowel signal following 2% DSS exposure (FIG. 6B, upper panel). It was observed that in vivo CD45-PET signal was significantly higher in both the large bowel (SUVₘₑₐₙ 3.2±1.51) and rectum (4.61±1.86) relative to controls (0.79±0.33, p<0.0001 and 0.69±0.26, p<0.0001, respectively) (FIGS. 13A and 13B). Further, there was a correlation between large bowel SUVₘₑₐₙ and weight loss in these mice (Pearson r=0.6675, p=0.0091) (FIG. 6C). However, no correlation was observed between rectum SUVₘₑₐₙ and weight loss (FIG. 13C).

Mice were then placed on a regular water regimen and subsequently randomized into dexamethasone (n=8, 1 mg/kg/day intraperitoneally for 3 days) and water-only groups (n=6). One water-only mouse died after 1 day of recovery and was excluded from all analyses below. After 8 days of recovery, PET/CT imaging was repeated. Ex vivo PET/CT imaging and gamma radiation assays were performed as previously described (FIG. 6A). Examination of in vivo PET images of the same mouse after 8 days of 2% DSS treatment and then again after 8 days of recovery revealed striking differences in probe uptake in the abdomen, with decreased signal in the large bowel and rectum (FIGS. 4D (upper panel), 13D, and 13E). Interval reduction in PET signal was visualized in the same segment of bowel between both timepoints (FIGS. 4B and 4D, lower panels).

In vivo analysis showed a notable decrease in signal intensity across both groups, with no statistically significant difference between the two (FIGS. 13F and 13G). No difference in weight gain was observed between mice placed on the water-only regimen (n=5) and those placed on water recovery with dexamethasone treatment (FIG. 13H). The degree of large bowel signal reduction robustly correlated with an improvement in disease status, represented by weight gain (Pearson r=−0.89, p<0.0001) (FIG. 6E). Correlation was not observed between rectum signal change and weight gain (FIG. 13I).

### Example 8: Human CD45-PET Probe Design and Function

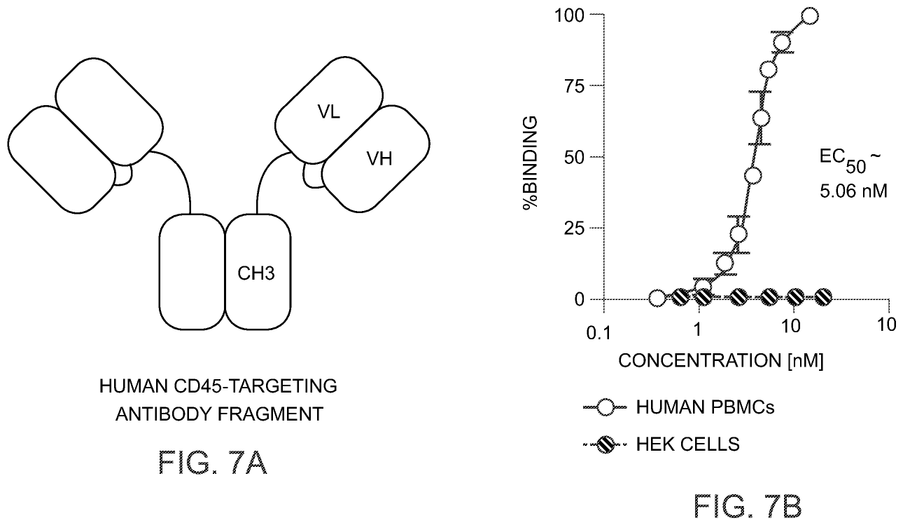

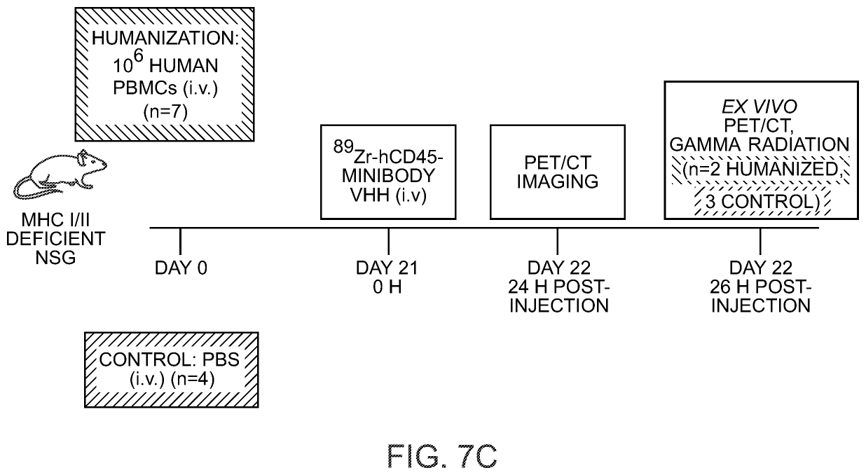

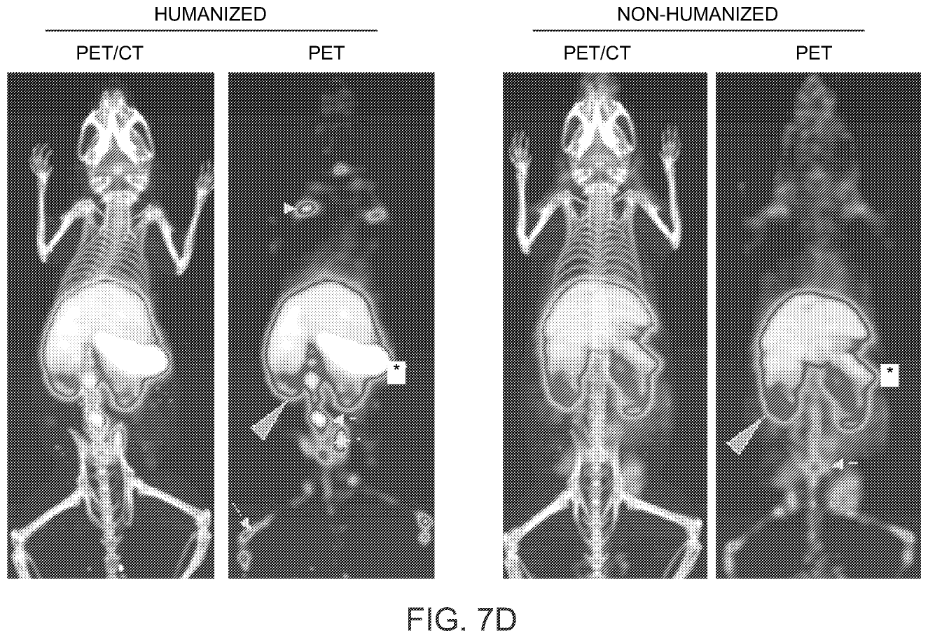

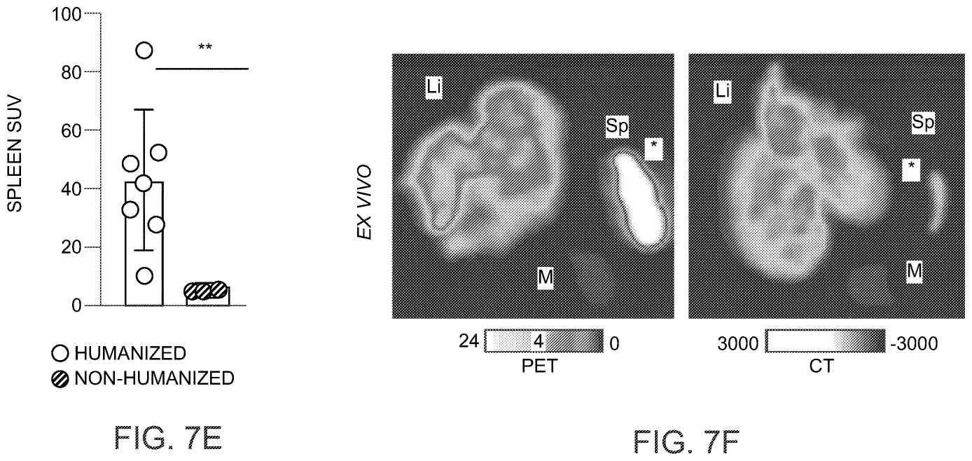

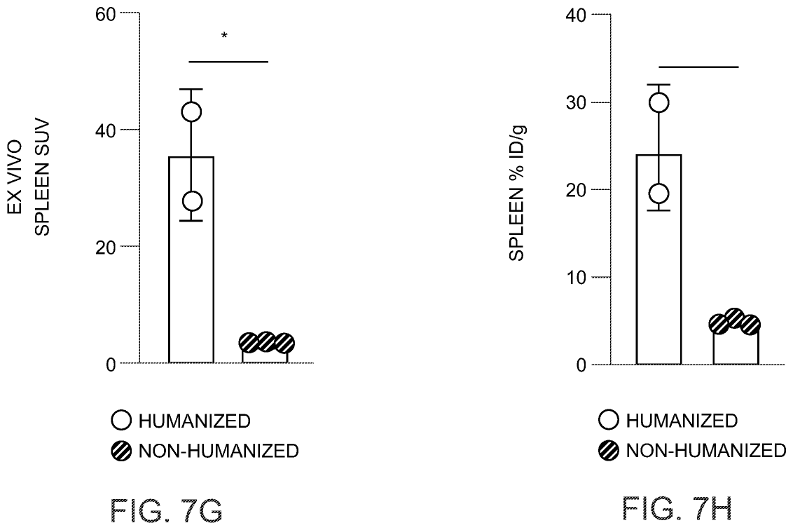

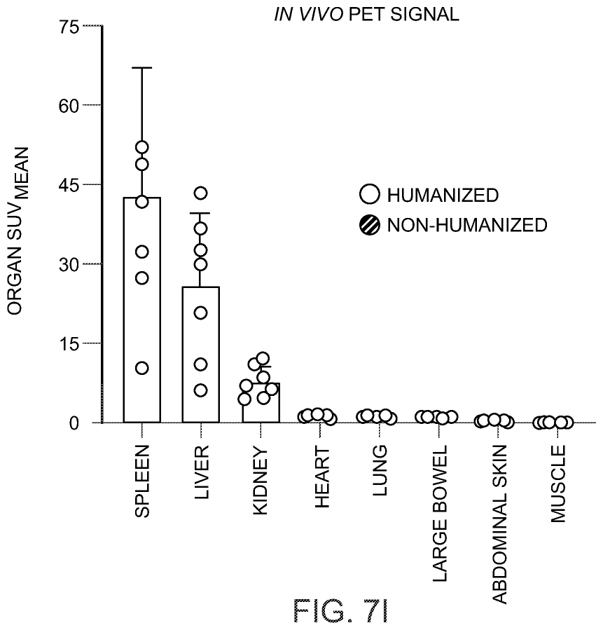

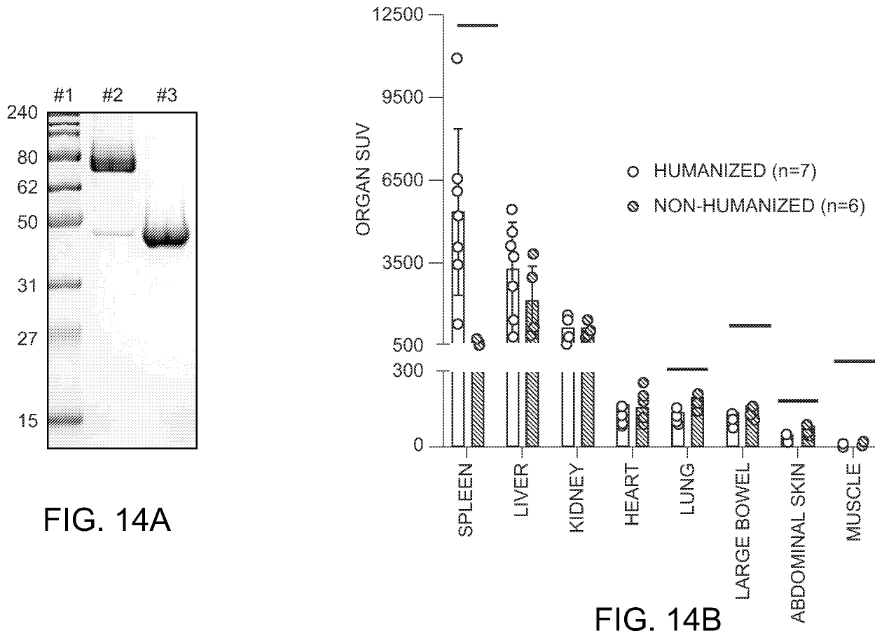

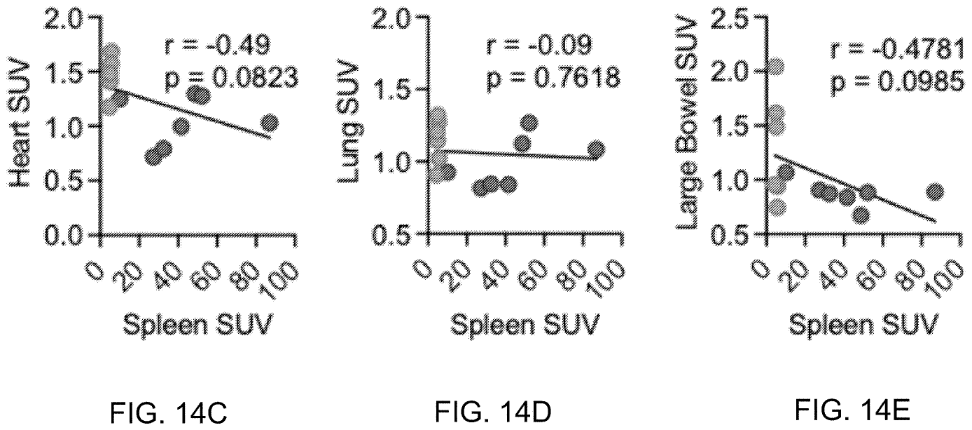

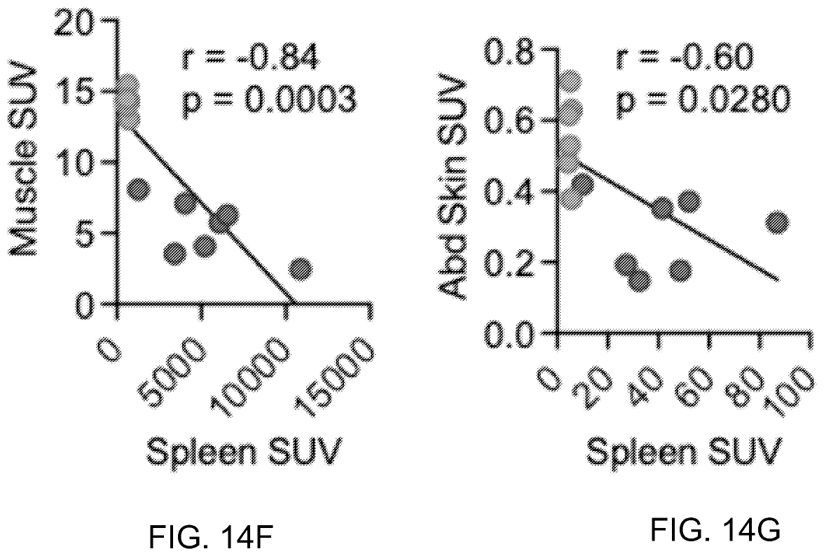

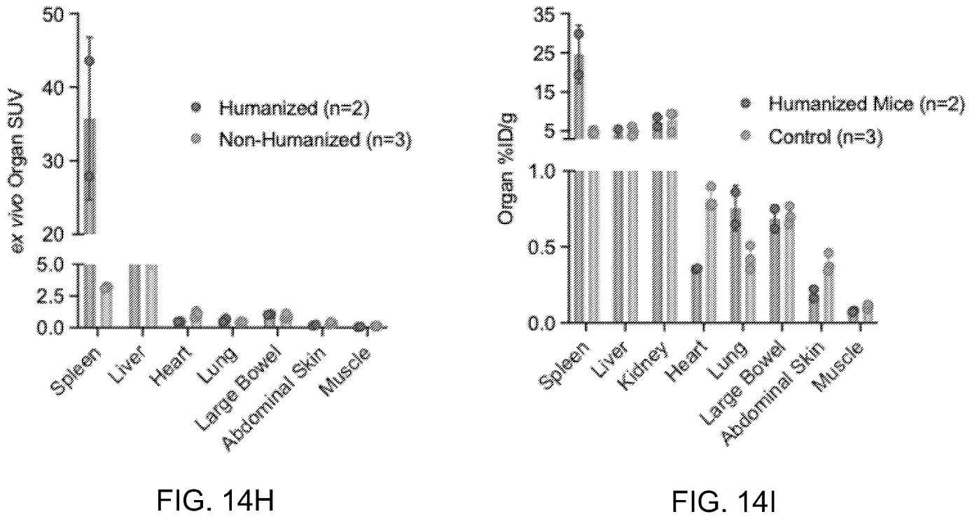

Designing a human CD45-PET probe. To adapt CD45-PET to a clinical setting, a PET probe specific to human CD45 was developed. A full-size CD45 antibody (clone BC8), which has been used in the clinic as a radiotherapeutic (Orozco et al., Blood 121(18):3759-3767 (2013); Vo et al., Blood 132(Supp 1):1018 (2013)), was engineered into a smaller antibody fragment. This alteration facilitates rapid clearance from the circulation, making it more suitable for PET imaging. The engineered fragment comprises the variable regions of immunoglobulin heavy (VH) and light chains (VL), along with the CH3 domain of IgG1, forming a stable dimer (~80 kDa in size) (FIG. 7A). The engineered antibody fragment, often referred to in the art as a “minibody” and referred to herein as the “human CD45-PET probe,” was produced in HEK293 cells with a yield of approximately 50 mg per liter of culture following standard lab techniques. Purification was achieved using Ni-NTA affinity column, followed by size-exclusion chromatography, with further verification through SDS-PAGE analysis (FIG. 14A). Subsequently, flow cytometric analysis was conducted to characterize the functionality of the human CD45-PET probe, evaluating its specificity and affinity for binding human immune cells. The probe exhibited high affinity for human peripheral blood mononuclear cells (PBMCs) with an EC₅₀ of ~5 nM with no binding to non-immune human cells, demonstrating its specificity and strong binding affinity (FIG. 7A).

Human CD45-PET probe detected human immune cells in humanized mice. To assess whether the developed human CD45-PET probe can detect inflammation in vivo, NOD.Cg-Prkdc^{scid} I12rg^{tmlWjl}/SzJ (NSG) mice, which are deficient in major histocompatibility complex (MHC) class I (H2-K and D) and II (IA) molecules (NSG-MHCI/II dKO), were injected with 10 million human PBMCs to allow for human immune cell engraftment. Three weeks after injection, mice underwent CD45-PET imaging using the ⁸⁹Zr-labeled human CD45-PET probe. The probe underwent modification with DFO and subsequent labeling with ⁸⁹Zr following established protocols (Vosjan et al., Nat. Protoc. 5(4):739-743 (2010)). Non-humanized NSG-MHCI/II dKO mice were also injected and imaged with the human CD45-PET probe to serve as controls. A portion of humanized (n=2) and non-humanized control (n=3) mice were sacrificed for ex vivo imaging and gamma radiation assay (FIG. 7C).

The human CD45-PET probe clearly detected engrafted human PBMCs in the spleen and bone marrow in humanized mice in vivo (FIG. 7D). The spleen signal in vivo was significantly higher than non-humanized mice (SUVₘₑₐₙ 1.18±26.0 vs 5.04±0.5, p=0.014) (FIG. 7D). Ex vivo imaging of 2 humanized mice confirmed the in vivo findings (FIG. 7E), with significantly increased spleen signal (SUVₘₑₐₙ 35.73±11.07 vs 3.18±0.09, p=0.011) (FIG. 7F, 7G). Gamma radiation quantification further confirmed these findings (FIG. 7H). The human CD45-PET probe showed very low signal in major non-immune organs such as the heart, lung, large bowel, skin, and muscle (FIG. 7I). Uptake into the liver and, to a lesser extent, the kidney was observed in both the humanized and non-humanized cohorts, suggesting, without being bound by theory, both renal and hepatic clearance mechanism for the human CD45-PET probe in mice (FIGS. 14B, 14H, and 14I). Interestingly, most non-immune organ systems showed slightly increased non-specific signal in the non-humanized control group (FIG. 14B). Except for the lung, probe uptake in most major organs was inversely correlated with spleen uptake (FIGS. 14C-14G). However, independent of spleen signal, all non-immune organs had very low background. These results demonstrate that the developed human CD45-PET probe detects human-specific inflammation in these organs.

Whole body visualization of human immune cell infiltration using human CD45-PET probe. To demonstrate that the human CD45-PET probe can detect pathogenic inflammation, a longitudinal imaging study was conducted on the cohort of humanized NSG-MHCI/II dKO mice to detect early trafficking and infiltration of engrafted human immune cells as part of a graft-versus-host disease (GVHD) process. To that end, it was investigated whether CD45-PET imaging could detect pathogenic human immune cell infiltration across major organ systems in humanized mice.

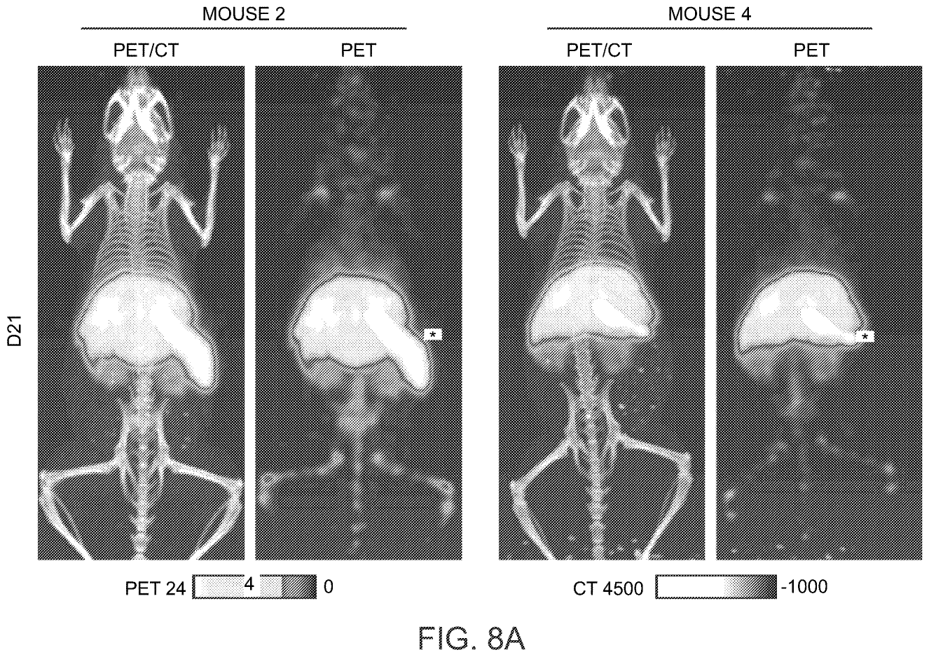

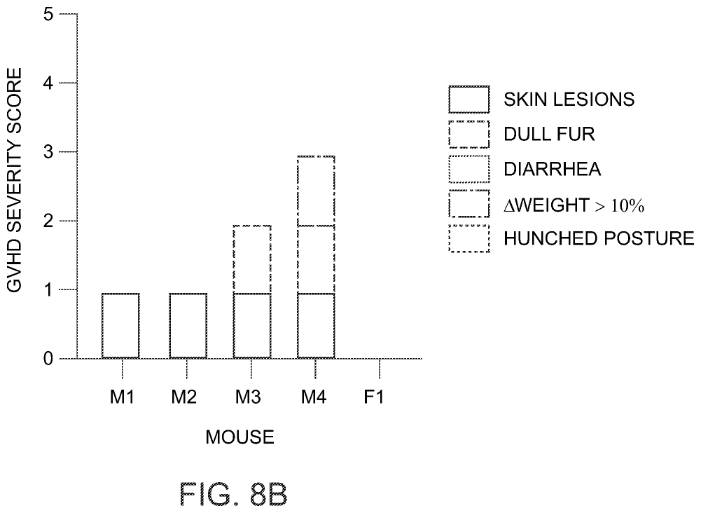

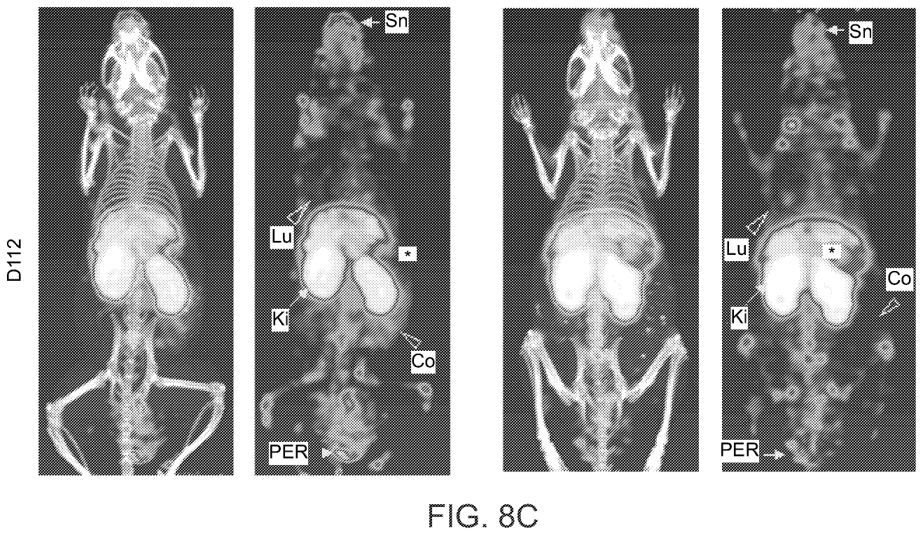

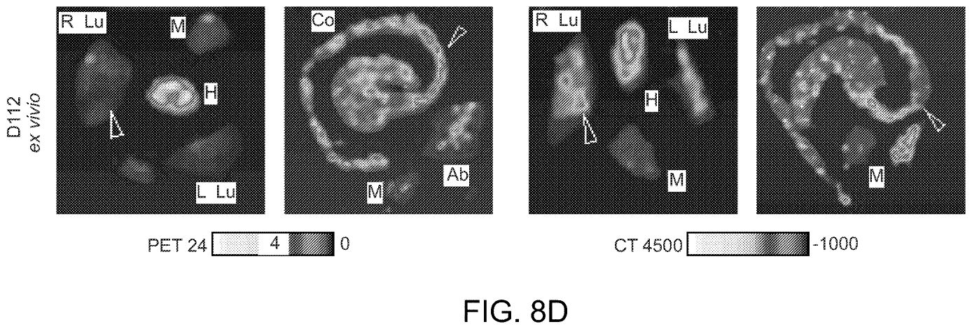

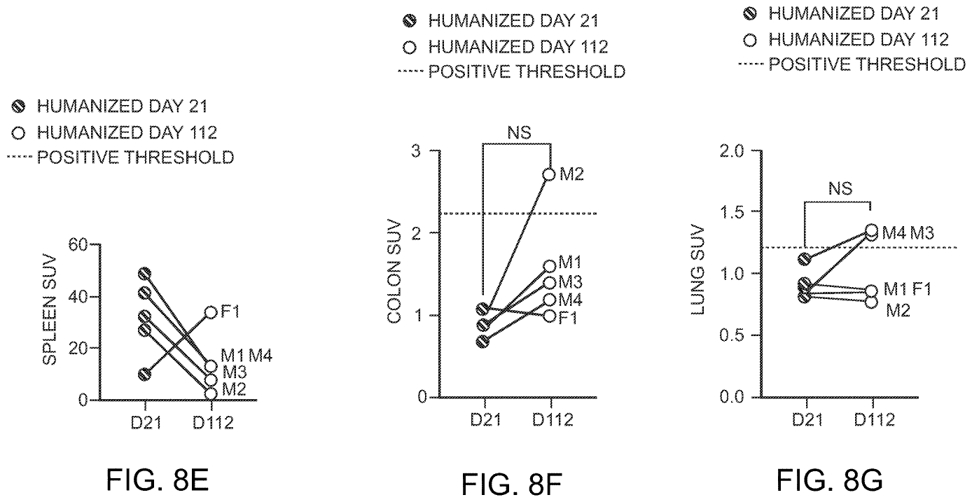

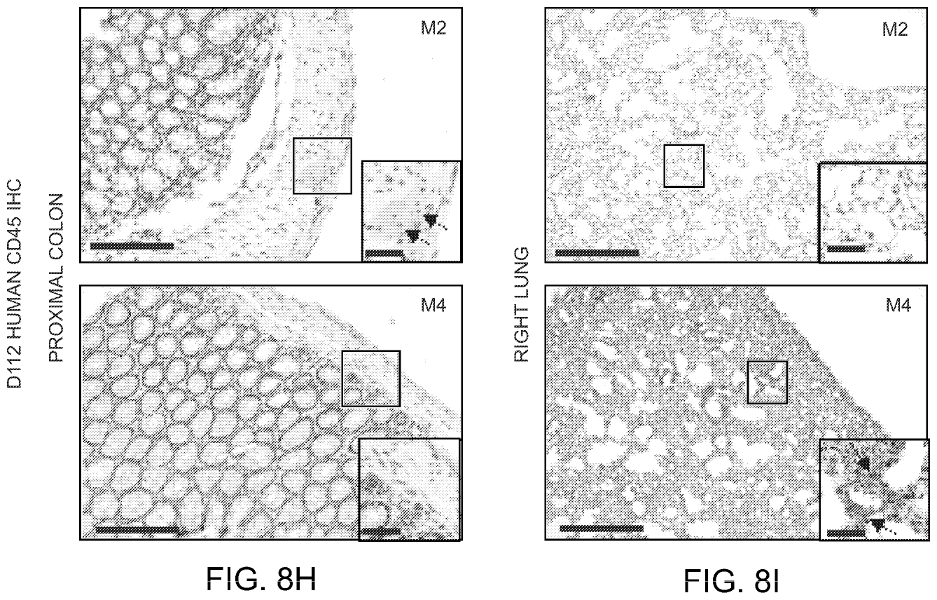

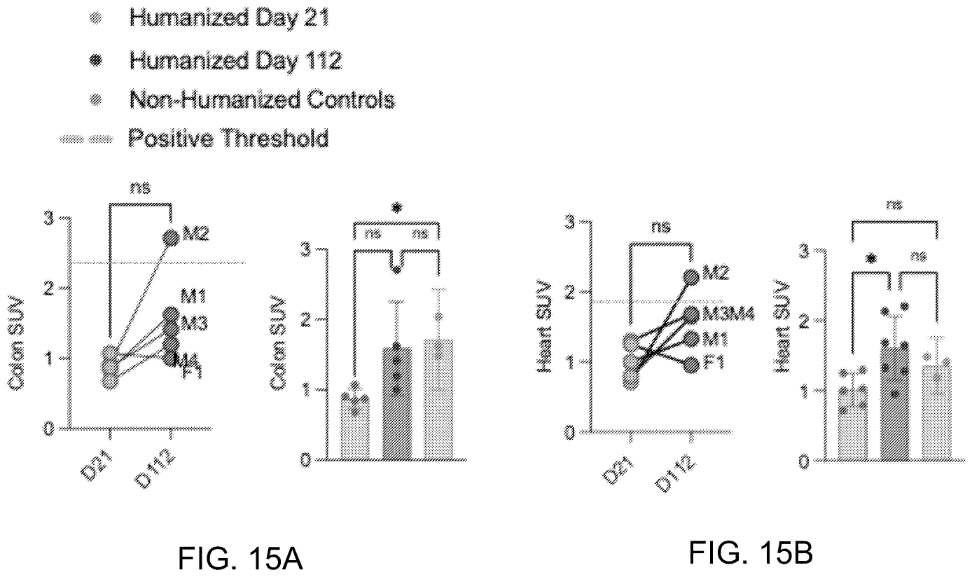

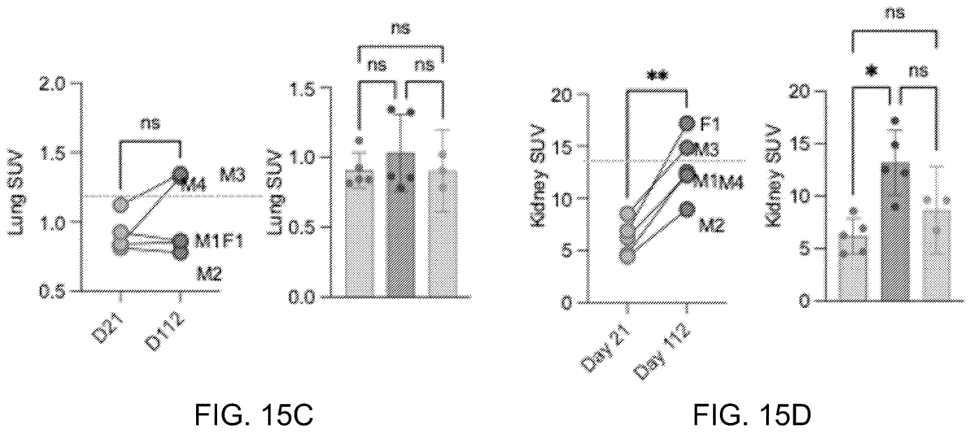

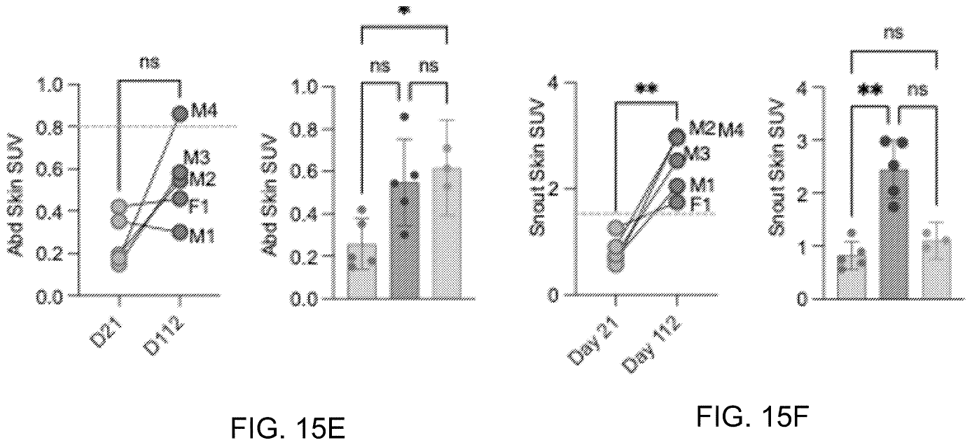

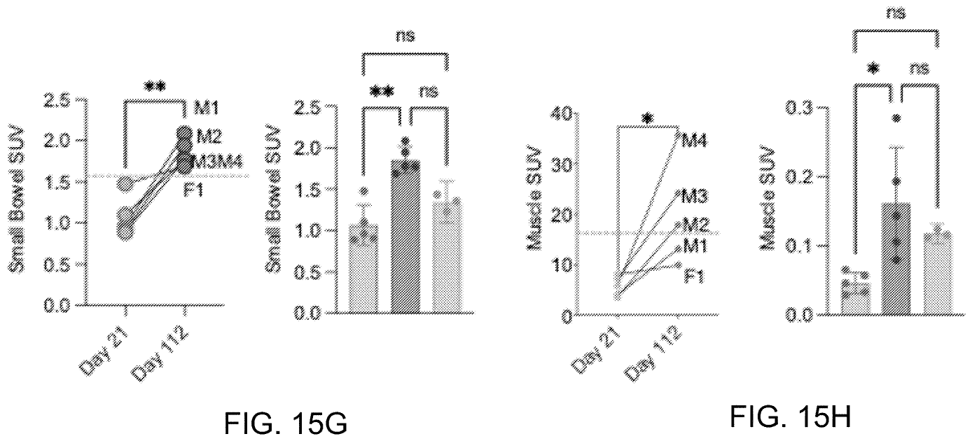

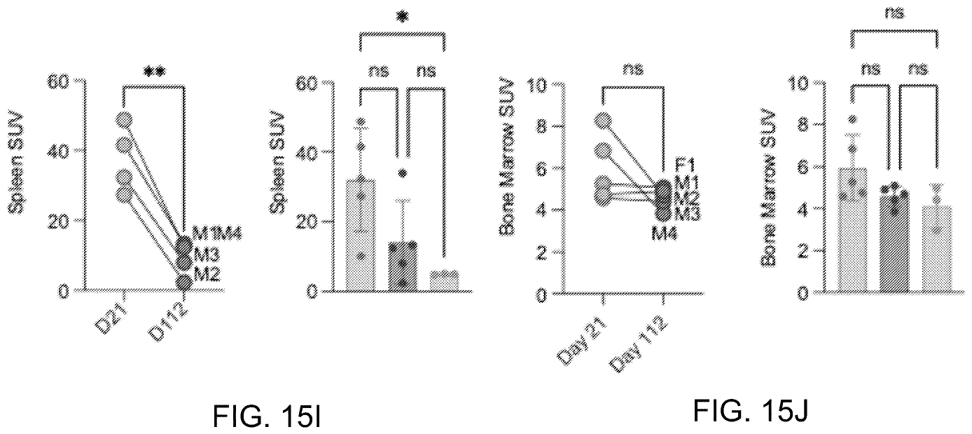

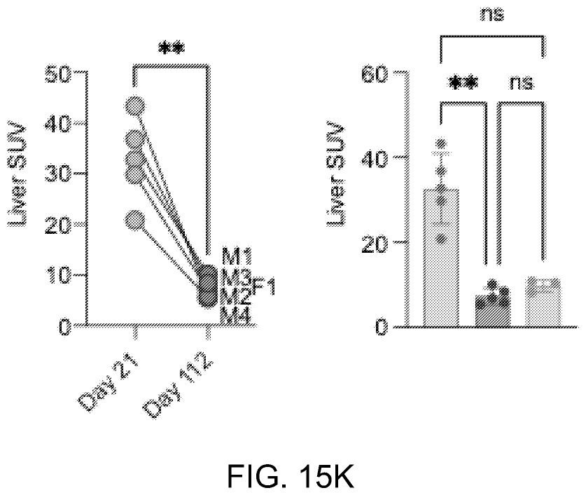

After undergoing their first round of CD45-PET imaging on Day 21 (FIG. 8A), five humanized mice were routinely evaluated for signs of GVHD (dermatitis, weight loss, fur dullness, posture changes, diarrhea) according to previously published criteria (Naserian et al., Front. Immunol. 9:10, pp. 1-9 (2018)). Four of the five mice developed one or more symptoms of GVHD by Day 112 (skin lesions (n=4), dull fur (n=2), weight loss >10% (n=1)) (FIG. 8B), at which point CD45-PET imaging was repeated (FIG. 8C) with subsequent ex vivo imaging of select organ systems (FIG. 8D). In vivo PET probe uptake was quantified in eleven major organs (colon, heart, lungs, kidneys, abdominal skin, snout skin, muscle, spleen, bone marrow, liver) and compared against Day 21 in vivo PET signal (FIG. 15).

Review of each mouse's imaging series revealed several key findings. All mice with one or more symptom of GVHD showed a significant reduction in their spleen uptake (p=0.0019) between Day 21 and Day 112 (FIGS. 8E and 15I). Most other organs showed increased probe uptake in most or all humanized mice at Day 112 (FIGS. 15A-15H), apart from femoral bone marrow and liver (FIGS. 15J-15K). However, each mouse displayed a unique set of changes in PET signal between both timepoints. For example, as shown in a side-by-side comparison of humanized mouse 2 (M2) and mouse 4 (M4), M2 showed increased large bowel signal localizing to the proximal colon, but M4 did not. The reverse was true for lung signal, where M4 showed subtle increases in the lung periphery while M2 did not (FIGS. 8C-6D).

To determine whether signal increases reflected non-specific signal secondary to spleen loss or true graft infiltration, the upper limit of the 95th percent confidence interval (95% CI) of the control signal for each organ was applied to each as a positive signal threshold, which allowed for detection of human immune cells on an organ-level (FIGS. 6F-6G and 15A-15 K). For example, while most mice had a slight increase in colon signal, only mouse 2 (M2) had colon signal (SUVₘₑₐₙ=2.70) that was above the positive signal threshold (SUVₘₑₐₙ=2.43) (FIG. 8F). Also, M4 lung signal (SUVₘₑₐₙ=1.34) reached the positive threshold (SUVₘₑₐₙ=1.20), while M2 did not (SUVₘₑₐₙ=0.78) (FIG. 8G). IHC staining for hCD45 showed that human CD45⁻ cells were present only in above-threshold organs (FIGS. 8H and 8I). Impressively, above-threshold PET uptake occurred in some tissues where there was a low level of hCD45⁺ infiltrate but no significant evidence of tissue damage (FIG. 8H, M2 proximal colon). However, in other tissues, evidence of tissue damage was observed, particularly with increased hCD45⁺ infiltrate (FIG. 8I, M4 lung). These data further verify the accuracy and diagnostic reliability of the disclosed CD45− PET probe and methods of use thereof.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A radiolabeled tracer, comprising:
a) a single chain variable antibody fragment (scFv) that binds CD45; wherein the scFv comprises a variable light (VL) domain and a variable heavy (VH) domain connected by a first linker;
b) a constant heavy (CH) 3 domain connected to the scFv by a second linker; and
c) a radioactive isotope detectable via positron emission tomography (PET) or single-photon emission computerized tomography (SPECT), wherein the radioactive isotope is connected to at least one of the VL, VH, and CH3 domains.

2. The radiolabeled tracer of claim 1, wherein the VH domain comprises the amino acid sequence QVQLVESGGGLVQPGGSLKLSCAASGFDFSRYWMSWVRQAPGKGLEWIGEI NPTSSTINFTPSLKDKVFISRDNAKNTLYLQMSKVRSEDTALYYCARGNYYR YGDAMDYWGQGTSVTVSKIS (SEQ ID NO: 9); and/or
wherein the VL domain comprises the amino acid sequence DIVLTQSPASLAVSLGORATISCRASKSVSTSGYSYLHWYQQKPGOPPKLLIY LASNLESGVPARFSGSGSGTDFTLNIHPVEEEDAATYYCQHSRELPFTFGSGTK LEIK (SEQ ID NO: 10); or
wherein the scFv comprises the amino acid sequence QVQLVESGGGLVQPGGSLKLSCAASGFDFSRYWMSWVRQAPGKGLEWIGEI NPTSSTINFTPSLKDKVFISRDNAKNTLYLOMSKVRSEDTALYYCARGNYYR YGDAMDYWGQGTSVTVSKISGGGGSGGGGSGGGGSGGGGSGGGGSSDIVLT QSPASLAVSLGQRATISCRASKSVSTSGYSYLHWYQQKPGQPPKLLIYLASNL ESGVPARFSGSGSGTDFTLNIHPVEEEDAATYYCQHSRELPFTFGSGTKLEIKG GGSEPKSPKSADKTHTAPQPREPQVYTLPPSRDELTKNQVSLTCLVKGFYPSD IAVEWESNGQPENNYKTTPPVLDSDGSFFLYSKLTVDKSRWQQGNVFSCSVM HEALHNHYTOKSLSLSPGK (SEQ ID NO: 33).

3. (canceled)

4. The radiolabeled tracer of claim 1, wherein the first linker, the second linker, or both the first and the second linkers comprise the amino acid sequence GGGX, GGGGX (SEQ ID NO: 12), GSSGSX (SEQ ID NO: 13), GGGGSGGGGSGGGGSGGGGS (SEQ ID NO: 18), GGGGSGGGGSGGGGSGGGGSGGGGS (SEQ ID NO: 19), GGGSEPKSPKSADKTHTAP (SEQ ID NO: 20), GSTSGSGKPGSGEGSTKG (SEQ ID NO: 21), KESGSVSSEQLAQFRSLD (SEQ ID NO: 22), EGKSSGSGSESKST (SEQ ID NO: 23), or GSAGSAAGSGEF (SEQ ID NO: 24), wherein X is either C or S.

5.-6. (canceled)

7. The radiolabeled tracer of claim 1, wherein the CH3 domain is connected to the VL domain of the scFv; or
wherein the CH3 domain is connected to the VH domain of the scFv; or
wherein the CH3 domain comprises the amino acid sequence QPREPQVYTLPPSRDELTKNQVSLTCLVKGFYPSDIAVEWESNGQPENNYKTT PPVLDSDGSFFLYSKLTVDKSRWQQGNVFSCSVMHEALHNHYTQKSLSLSPG K (SEQ ID NO: 11).

8.-9. (canceled)

10. The radiolabeled tracer of claim 1, wherein the radioactive isotope is directly connected to an amino acid residue of at least one of the VL, VH, and CH3 domains; or wherein the radioactive isotope is connected to at least one of the VL, VH, and CH3 domains via a chelator.

11. (canceled)

12. The radiolabeled tracer of claim 10, wherein the chelator comprises desferrioxamine (DFO), dodecane tetraacetic acid (DOTA), or 1,4,7-Triazacyclononane-1,4,7-triacetic acid (NOTA).

13. The radiolabeled tracer of claim 1, wherein the radioactive isotope is zirconium-89 (⁸⁹Zr), fluorine-18 (¹⁸F), iodine-124 (¹²⁴I), copper-64 (⁶⁴Cu), gallium-68 (⁶⁸Ga), yttrium-90 (⁹⁰Y), or indium-111 (¹¹¹In).

14. The radiolabel tracer of claim 1, wherein the radioactive isotope is detectable via PET.

15. The radiolabeled tracer of claim 14, wherein the radioactive isotope is ⁸⁹Zr or Copper-64(64Cu).

16. (canceled)

17. The radiolabel tracer of claim 1, wherein the radioactive isotope is detectable via SPECT.

18. The radiolabeled tracer of claim 17, wherein the radioactive isotope is indium-111 (¹¹¹In) or technetium 99m (^{99m}Tc).

19. (canceled)

20. The radiolabeled tracer of claim 1, comprising a polyethylene glycol (PEG) molecule.

21. The radiolabeled tracer of claim 20, wherein the PEG molecule has a molecular weight of about 10 kilodaltons (kDa) or about 20 kDa; and
wherein the PEG molecule is connected to at least one of the VL, VH, and CH3 domains.

22. (canceled)

23. The radiolabeled tracer of claim 1, which is in the form of a dimer.

24. A diagnostic composition comprising an effective amount of the radiolabeled tracer of claim 1 and a pharmaceutically acceptable carrier.

25. A method of detecting inflammation in a subject, comprising:
intravenously administering to a subject in need thereof, the diagnostic composition of claim 24; and
producing a first image of a tissue of the subject by PET or SPECT imaging;
wherein detecting the radioactive isotope in the tissue is an indication of inflammation in the tissue.

26. The method of claim 25, comprising producing the first image about 2 hours to about 96 hours, about 2 hours to about 24 hours, or about 2 hours to about 5 hours after administering the diagnostic composition or on the same on the same calendar day as administering the diagnostic composition.

27.-29. (canceled)

30. The method of claim 25, comprising producing the first image by PET imaging which is PET-computed tomography (PET-CT) or PET-magnetic resonance imaging (PET-MRI); or
comprising producing the first image by SPECT imaging.

31.-32. (canceled)

33. The method of claim 25, further comprising detecting immune cell infiltrates in the first image.

34. The method of claim 25, wherein the tissue is cardiac tissue, lung tissue, liver tissue, kidney tissue, digestive tissue, skin tissue, nervous system tissue, or bladder tissue.

35. (canceled)

36. The method of claim 34, wherein the tissue is cardiac tissue and the method further comprises testing the subject for a non-inflammatory cardiomyopathy in the event that the first image shows no radiolabeled tracer in the cardiac tissue.

37. The method of claim 36, wherein the subject is suspected of having an inflammatory cardiomyopathy; or
wherein the inflammatory cardiomyopathy is myocarditis, cardiac sarcoidosis, or cellular cardiac transplant rejection.

38. (canceled)

39. The method of claim 37, wherein the first image is taken of the myocardium.

40. The method of claim 39, wherein the first image is taken of the heart.

41. (canceled)

42. The method of claim 34, wherein the tissue is digestive tissue and wherein the subject is suspected of having an inflammatory bowel disease (IBD); or
wherein the tissue is lung tissue and wherein the subject is suspected of having a pulmonary inflammatory disease; or
wherein the tissue is at least one of skin tissue, liver tissue, digestive tissue, and lung tissue.

43. The method of claim 42, wherein the IBD is eosinophilic esophagitis, chronic gastroesophageal reflux, Helicobacter pylori infection, Crohn's disease, or ulcerative colitis; or
wherein the pulmonary inflammatory disease is acute respiratory distress syndrome (ARDS), chronic obstructive pulmonary disease (COPD), pneumonia, or an interstitial lung disease; or
wherein the tissue is at least one of skin tissue, liver tissue, digestive tissue, and lung tissue and wherein the subject is suspected of having graph-versus-host disease; or
wherein the subject is suspected of having a fever of unknown origin, or vasculitis.

44. The method of claim 43, wherein the first image is taken of the large bowel, the rectum, anal canal, or a combination of two or more thereof.

45.-50. (canceled)

51. The method of claim 25, wherein the subject has been diagnosed with coronavirus infection or disease; or
wherein the detecting comprises quantifying the amount of the radioactive isotope in the first image.

52. (canceled)

53. The method of claim 25, further comprising
administering intravenously to the subject, an effective amount of the diagnostic composition after the first image is produced; and
producing a second image of the tissue in the subject.

54. The method of claim 53, comprising producing the second image at least two weeks after producing the first image; or
comprising comparing the first and the second images; or
comprising quantifying the amount of the PET isotope in the second image; or
comprising comparing the quantified amount of PET isotope in the first and the second images.

55.-57. (canceled)

58. A method detecting the presence of CD45⁺ immune cells in a tissue of a subject, comprising:
intravenously administering to a subject in need thereof, the diagnostic composition of claim 24; and
producing a first image of a tissue of the subject by PET or SPECT imaging;
wherein detecting the radioactive isotope in the tissue is an indication of the presence of CD45⁺ immune cells.

59. The method of claim 58, wherein the tissue is cardiac tissue, lung tissue, liver tissue, kidney tissue, digestive tissue, nervous system tissue, or bladder tissue.

60. A kit, comprising:
a) an effective amount of the radiolabeled tracer of claim 1; and
b) printed instructions for use in intravenously administering the radiolabeled tracer to a subject having or suspected of having inflammation or an inflammatory disease or condition.

61. (canceled)

62. A method of treating an inflammatory disease in a subject comprising:
imaging a subject in need thereof by the method of claim 25; and
if the radiolabeled tracer is detected, administering an appropriate therapy to the subject.

63. The method of claim 62, wherein the inflammatory disease is an inflammatory cardiomyopathy, an IBD, a pulmonary inflammatory disease, or graph-versus-host disease.


SEQUENCE LISTING (41 sequences)

SEQ ID NO: 1 - protein, 111 aa, synthetic construct
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 2 - protein, 123 aa, synthetic construct
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAK

SEQ ID NO: 3 - protein, 111 aa, synthetic construct
DIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVE

SEQ ID NO: 4 - protein, 119 aa, synthetic construct
LSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSS

SEQ ID NO: 5 - protein, 111 aa, synthetic construct
DIVMTQTPASVEAAVGGTVTINCQASQSISSWLSWYQQKPGQPPKLLIYGASNLASGVPSRFSGSGSGTQFSLTISDLECADAATYYCQSYYDSGSSVFFNFGGGTKVVVK

SEQ ID NO: 6 - protein, 119 aa, synthetic construct
QSLEESGGDLVKPGASLTLTCTASGFSFSAGYWICWVRQAPGKGLEWIACIYAGSSGSTYYASWAKGRFTIPKTSSTTVTLQMTSLTGADTATYFCARGNAGVAVGALWGPGTLVTVSS

SEQ ID NO: 7 - protein, 107 aa, synthetic construct
DIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVK

SEQ ID NO: 8 - protein, 120 aa, synthetic construct
QEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSS

SEQ ID NO: 9 - protein, 123 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKIS

SEQ ID NO: 10 - protein, 111 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 11 - protein, 106 aa, Homo sapiens [sequence omitted]

SEQ ID NO: 12 - protein, 5 aa, synthetic construct
GGGGX

SEQ ID NO: 13 - protein, 6 aa, synthetic construct
GSSGSX

SEQ ID NO: 14 - protein, 5 aa, synthetic construct
GGGGS

SEQ ID NO: 15 - protein, 5 aa, synthetic construct
GSPRG

SEQ ID NO: 16 - protein, 10 aa, synthetic construct
GGGGSGGGGS

SEQ ID NO: 17 - protein, 15 aa, synthetic construct
GGGGSGGGGSGGGGS

SEQ ID NO: 18 - protein, 20 aa, synthetic construct
GGGGSGGGGSGGGGSGGGGS

SEQ ID NO: 19 - protein, 25 aa, synthetic construct
GGGGSGGGGSGGGGSGGGGSGGGGS

SEQ ID NO: 20 - protein, 20 aa, synthetic construct
SGGSGGGGSGGGSGGGGSLQ

SEQ ID NO: 21 - protein, 18 aa, synthetic construct
GSTSGSGKPGSGEGSTKG

SEQ ID NO: 22 - protein, 18 aa, synthetic construct
KESGSVSSEQLAQFRSLD

SEQ ID NO: 23 - protein, 14 aa, synthetic construct
EGKSSGSGSESKST

SEQ ID NO: 24 - protein, 12 aa, synthetic construct
GSAGSAAGSGEF

SEQ ID NO: 25 - protein, 9 aa, Homo sapiens
QSFGLLDPK

SEQ ID NO: 26 - protein, 23 aa, Homo sapiens
LSEGDKVKMDSRIQVLSRGVNQT

SEQ ID NO: 27 - protein, 49 aa, Homo sapiens
KPTTTPAPRPPTPAPTIASQPLSKRPEACRPAAGGAVHTRGLDFACDIY

SEQ ID NO: 28 - protein, 39 aa, Homo sapiens
IEVMYPPPYLDNERSNGTIIHVKGKHLCPSPLFPGPSKP

SEQ ID NO: 29 - protein, 22 aa, Homo sapiens
EPKSCDKTHTCPPCPAPELLGG

SEQ ID NO: 30 - protein, 18 aa, Homo sapiens
ERKCCVECPPCPAPPAAA

SEQ ID NO: 31 - protein, 19 aa, Homo sapiens
ESKYGPPCPPCPAPEFLGG

SEQ ID NO: 32 - protein, 260 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 33 - protein, 385 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGSEPKSPKSADKTHTAPQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 6 aa, synthetic construct
DTYRYI

SEQ ID NO: 35 - protein, 6 aa, synthetic construct
TDFYLK

SEQ ID NO: 36 - protein, 6 aa, synthetic construct
HHHHHH

SEQ ID NO: 37 - protein, 7 aa, synthetic construct
DYKDDDK

SEQ ID NO: 38 - protein, 24 aa, synthetic construct
EFDYKDDDDKGSLPETGGHHHHHH

SEQ ID NO: 39 - protein, 19 aa, synthetic construct
KDHLIHNVHKEFHAHAHNK

SEQ ID NO: 40 - protein, 8 aa, synthetic construct
QPELAPED

SEQ ID NO: 41 - protein, 5 aa, synthetic construct
LPETG